(19) United States
(12) Patent Application Publication
(10) Pub. No.: US 2024/0293553 A1
(43) Pub. Date: Sep. 5, 2024

(54) P-SELECTIN INHIBITORS AND USES THEREOF

(71) Applicant: Beth Israel Deaconess Medical Center, Inc., Boston, MA (US)

(72) Inventors: Elliot Chaikof, Newton, MA (US); Appi Mandhapati, Boston, MA (US); Simon Park, Boston, MA (US); Richard D. Cummings, Brookline, MA (US)

(73) Assignee: Beth Israel Deaconess Medical Center, Inc., Boston, MA (US)

(21) Appl. No.: 18/693,494

(22) PCT Filed: Sep. 22, 2022
(86) PCT No.: PCT/US2022/044389

§ 371 Date: Mar. 19, 2024

Related U.S. Application Data
(60) Provisional application No. 63/247,681, filed on Sep. 23, 2021.

(51) Int. Cl. A61K 47/54; A61K 47/60; A61P 7/02

(52) U.S. Cl. CPC A61K 47/545; A61K 47/549; A61K 47/60; A61P 7/02

(57) ABSTRACT

Provided are novel P-selectin inhibitors, compositions and uses thereof, and methods of making thereof.

### CROSS-REFERENCE TO RELATED APPLICATIONS

This application claims the benefit of and priority to U.S. Provisional Application No. 63/247,681, filed Sep. 23, 2021, titled P-SELECTIN INHIBITORS AND USES THEREOF, the contents of which are incorporated herein by reference in their entirety.

### FEDERALLY SPONSORED RESEARCH

This invention was made with government support under Grant Numbers HL128237, GM116196, and DK107405, awarded by the National Institutes of Health. The government has certain rights in the invention.

### BACKGROUND OF INVENTION

The selectin family of cell adhesion molecules, together with their glycoconjugate ligands, participate in leukocyte trafficking to sites of inflammation and to lymphoid organs. P- and E-selectins are expressed on activated vascular endothelial cells where they mediate initial tethering and rolling of leukocytes on endothelial cells by binding to P-selectin glycopeptide ligand-1 (PSGL-1) present on the surface of leukocytes. P-selectin is also expressed on activated platelets. L-selectin is expressed on the surface of leukocytes and mediates leukocyte-leukocyte interactions by binding to PSGL-1 present on the surface of other leukocytes promoting leukocyte accumulation to the inflammatory sites. Selectins recognize the sialyl Lewis x epitope (Slex or sLe^{x} NeuAca2-3Galpi-4(Fucal-3)GlcNAcpi-) on glycoconjugate ligands. However, selectin binding to SLex determinant alone is low affinity and is necessary but not sufficient for physiological interactions. Thus, selectins require additional post-translational modifications or peptide components for high-affinity binding to their ligands. P- and L-selectin both bind to the extreme N-terminus of PSGL-1 and interact with three clustered tyrosine sulfate residues and a nearby core-2-based O-glycan with sialyl Lewis x epitope (C2-SLex). The N-terminus of human PSGL-1 contains three potential tyrosine sulfation sites (Y46, Y48 and Y51) and two potential O-glycan attachment sites (T44 and T57).

Several small molecule inhibitors and protein therapeutics aimed at blocking PSGL-1/P-selectin interactions are already in clinical trials. Certain candidates pose production, stability, and immunity issues. Thus, there is a need for molecules that bind to selectins with high specificity and affinity, which in turn inhibit selectin mediated cell-cell interactions that have desirable pharmacological properties.

### SUMMARY OF INVENTION

In one aspect, provided herein is a glycopeptide, or a salt thereof, comprising the formula Y¹X¹Y²X²X³Y³X⁴X⁵X⁶Z¹X⁷W¹ (SEQ ID NO: 1), wherein:
W¹ is threonine or serine conjugated with a saccharide or polysaccharide via a linker L¹;
L¹ comprises a substituted or unsubstituted aryl group or a substituted or unsubstituted heteroaryl group;
X¹, X², X³, X⁴, X⁵, X⁶, and X⁷ are each individually and independently any amino acid;
Y¹, Y², and Y³ are each individually and independently tyrosine, phenylalanine, or phenylglycine, and wherein Y¹, Y², and Y³ are each independently unsubstituted or substituted with —SO₃H, —CH₂SO₃H, —CF₂SO₃H, —CO₂H, —CONH₂, —NHSO₂CH₃, —SO₂NH₂, or —CH₂PO₃H;
wherein at least one of Y¹, Y², and Y³ is substituted with —CH₂SO₃H; and
Z¹ is proline or hydroxyproline.

In certain embodiments, the polysaccharide is of the formula:

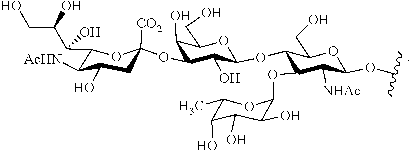

In certain embodiments, the glycopeptide comprises a sequence selected from: Y¹EY²LDY³DFLZ¹EW¹ (SEQ ID NO: 2), Y¹EY²LDY³DFLZ¹EW¹EP (SEQ ID NO: 3), Y¹EY²LDY³DFLZ¹EW¹EPL (SEQ ID NO: 4), EY¹EY²LDY³DFLZ¹EW¹ (SEQ ID NO: 5), EY¹EY²LDY³DFLZ¹EW¹E (SEQ ID NO: 6), EY¹EY²LDY³DFLZ¹EW¹EP (SEQ ID NO: 7), EY¹EY²LDY³DFLZ¹EW¹EPL (SEQ ID NO: 8), KEY¹EY²LDY³DFLZ¹EW¹ (SEQ ID NO: 9), KEY¹EY²LDY³DFLZ¹EW¹E (SEQ ID NO: 10), KEY¹EY²LDY³DFLZ¹EW¹EP (SEQ ID NO: 11), and KEY¹EY²LDY³DFLZ¹EW¹EPL (SEQ ID NO: 12).

In certain embodiments of the glycopeptide, or salt thereof, L¹ comprises a substituted or unsubstituted phenyl group. In certain embodiments of the glycopeptide, or salt thereof, L¹ comprises a substituted or unsubstituted triazole group.

In certain embodiments of the glycopeptide has the structure:

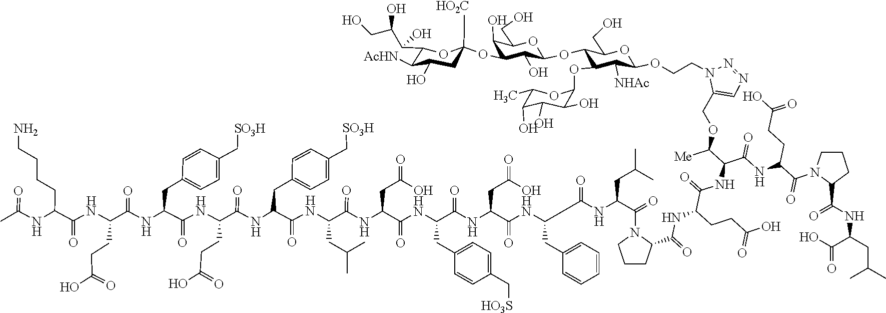

or a salt thereof.

In certain embodiments of the glycopeptide has the structure:

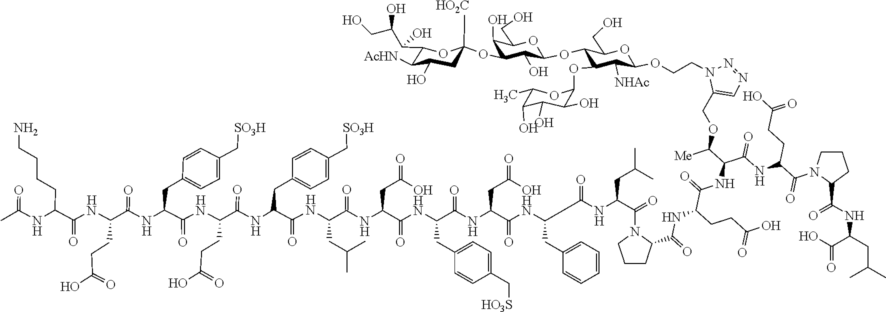

or a salt thereof; wherein n is 1-10,000.

In another aspect, provided herein is a pharmaceutical composition comprising a glycopeptide, or salt thereof, as described herein, and a pharmaceutically acceptable excipient. In certain embodiments, the pharmaceutical composition further comprises an additional therapeutic agent.

In another aspect, provided herein is a method, comprising administering to a subject a glycopeptide, or a salt thereof, as described herein, or a pharmaceutical composition as described herein.

In another aspect, provided herein is a method of inhibiting P-selectin binding to PSGL-1, comprising contacting the P-selectin with a glycopeptide, or salt thereof, as described herein.

In another aspect, provided herein is a method of treating or preventing cardiovascular disease, atherosclerosis, atherosclerotic lesions, thrombus formation, thromboembolism, stroke, or myocardial infarction in a subject in need thereof, comprising administering to the subject an effective amount of a glycopeptide, or a salt thereof, as described herein, or a pharmaceutical composition as described herein.

In another aspect, provided herein is a method of treating or preventing cancer metathesis, lupus, or an inflammatory disorder in a subject in need thereof, comprising administering to the subject an effective amount of a glycopeptide, or a salt thereof, as described herein, or a pharmaceutical composition as described herein.

In another aspect, provided herein is a method of thromboprophylaxis, comprising administering to a subject diagnosed with cancer an effective amount of a glycopeptide, or a salt thereof, as described herein, or a pharmaceutical composition as described herein.

In another aspect, provided herein is a method of treating or preventing allergy or lung disease in a subject in need thereof, comprising administering to the subject an effective amount of a glycopeptide, or a salt thereof, as described herein, or a pharmaceutical composition as described herein.

In another aspect, the glycopeptide, or salt thereof, as described herein, further comprises an imaging moiety. e.g., a radioisotope, or a dye. In certain embodiments, the radioisotope is ¹¹C, ¹³N, ¹⁵O, ¹⁸F, ⁶⁴Cu, ⁶²Cu, ¹²⁴I, ⁷⁶Br, ⁸²Rb, ⁸⁹Zr or ⁶⁸Ga. In certain embodiments, the radioisotope is ^{99m}Tc, ¹¹¹In, ¹²³I, or ²⁰¹Tl. In certain embodiments, the imaging moiety is a dye or a quantum dot, e.g., Kodak X-SIGHT Dyes and Conjugates, Pz 247, DyLight 750 and 800 Fluors, Cy 5.5 and 7 Fluors, Alexa Fluor 680 and 750 Dyes, IRDye 680 or 800CW Fluors. In a related aspect, provided herein is a method of imaging a subject, comprising administering to the subject a glycopeptide, or salt thereof, as disclosed herein, which further comprises an imaging agent, exposing the subject to imaging radiation, and acquiring an image of the subject.

In another aspect, provided herein is a method of making a glycopeptide, comprising reacting an polyaccharide group comprising a first reactive moiety, with a peptide comprising a second reactive moiety, to obtain the glycopeptide;
wherein the first reactive moiety and the second reactive moiety react to form a triazole containing moiety.

In another aspect, provided herein is a kit comprising a glycopeptide, or salt thereof, as described herein, and instructions for use.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, the Drawings, the Examples, and the Claims.

### BRIEF DESCRIPTION OF DRAWINGS

FIG. 1. Atomic charges for linker molecules in analog ligands 1 to 8.

FIG. 2. The positional RMSD for 0-link threonine in ligand analogs with reference to its position in a co-crystal structure during the MD simulations. The simulation in each replica was performed for 200 ns.

FIG. 3. Positional RMSD of the oligosaccharide together with middle tyrosine sulfate (left) and other residues in the peptide (right) in the P-selectin complexes with GSnP-6 (FIGS. 3A-3B), and 1 (FIGS. 3C-3D) relative to their initial positions over the course of MD simulations. The simulation in each replica was performed for 200 ns.

FIG. 4. Structure and HPLC profile of propargyl sulfonated peptide.

FIG. 5. Structure and HPLC profile of G4.

FIGS. 6A-6B. FIG. 6A shows structure and HPLC profile of P-G4 (n=903). FIG. 6B shows MALDI profile of P-G4. Reflector positive ion mode with Super DHB matrix.

FIG. 7. Validation of quantifying peptide detected in plasma by LC-MS/MS. Extracted ion chromatograms (left panels) of the quantifying peptide detected in plasma isolated from mice that were administered IV injections of (FIG. 7A) P-G4, (FIG. 7B) saline, or (FIG. 7C) no injection. Mass spectra (right panels) at 22.8 min (mass range 1110-1130 m/z). NL, normalization level (base peak intensity).

FIG. 8. Candidate N-terminal PSGL-1 glycomimetic analogues with targeted replacement of the T antigen (red). Triazole based linkers formed by copper (CuAAC) (1, 6, 7, 8) and strain-promoted (SPAAC) (2,3,4,5) azide-alkyne cycloaddition.

FIGS. 9A-9E. Structural overlay of G4, GSnP-6, and PSGL-1 bound to P-selectin (asterisked surface). FIG. 9A show structures most similar to the coordinate average shape of G4, GSnP-6, PSGL-1 from the MD simulations. Sulfated/sulfonated tyrosine positions in the ligand peptides are colored in green. FIG. 9B shows the orientations of tyrosine sulfates/sulfonates in the ligand peptides. Residues forming stable hydrogen bonds with the middle tyrosine sulfate/sulfonate are shown and labeled. Carbohydrate moieties for G4, GSnP-6, and PSGL-1 are shown in FIGS. 9C-9E, respectively. Backbones of the O-linked threonine in each ligand are shown as cartoon representation. Side chains of O-link threonine, GalNAc, and click-moiety are shown in licorice representation. The monosaccharide identities are shown with 3D-SNFG icons (Fuc, asterisked cone; GlcNAc, cube marked with arrow; Gal, encircled sphere; Neu5Ac, octahedron marked with karot) inside each ring. For simplicity, the Gal residue attached to GalNAc is not shown in GSnP-6 or PSGL-1.

FIGS. 10A-10C. Synthesis of G4 and P-G4. FIG. 10A shows chemical synthesis of 2-azidoethyl sLe^{X} (19) following sequential stereoselective glycosylations. FIG. 10B shows solid phase peptide synthesis of a propargyl peptide (20) and FIG. 10C shows copper (I)-catalyzed alkyne-azide cycloaddition of 19 and 20 to form G4 followed by PEGylation of G4 with mPEG-SVA (n=903) to generate P-G4.

FIGS. 11A-11C. G4 and P-G4 inhibit P-selectin binding to leukocytes. (FIG. 11A shows microarray binding studies of glycopeptide mimics towards human P-selectin (5 μg/mL), HECA-452, a monoclonal antibody specific to sLe^{x}, CHO131, a monoclonal antibody specific to the Core 2 O-glycan terminated sLe^{x}, or PL1, a monoclonal antibody specific to the N-terminal PSGL-1 peptide sequence. The compounds printed on the microarray are summarized in the table. Reference compounds included sialyl Lewis x (sLe^{x}), the biantennary glycans NA2, NA2,3, NA2,6, as well as lacto-N-neo-tetraose (LNnT) and biotin.

FIG. 11B shows surface plasmon resonance binding analysis to human P-selectin of GSnP-6 and G4. FIG. 11C shows G4 and P-G4 (0-100 μM) were incubated with mouse or human neutrophils and monocytes. Flow cytometry was used to evaluate the percent binding inhibition of species appropriate P-selectin chimera to neutrophils or monocytes produced by G4 and P-G4 as compared to phosphate buffered saline control. Both G4 and P-G4 inhibit P-selectin leukocyte interactions in a dose-dependent manner. Data are represented as mean±SEM, n=3/agent/study.

FIGS. 12A-12B. G4 and P-G4 inhibition of platelet-leukocyte aggregation. FIGS. 12A-12B shows inhibition of platelet-leukocyte aggregation in vitro. Anticoagulated mouse or human blood was dosed with 120 μM G4 and P-G4 or saline control. Platelet-leukocyte aggregation was induced by adding species-specific PAR peptide and samples were analyzed using flow cytometry to quantify platelet-positive monocytes or neutrophils. G4 and P-G4 significantly reduced platelet-leukocyte aggregation in both mouse as shown in FIG. 12A and human as shown in FIG. 12B blood. Unstimulated control blood is included for reference.

FIGS. 13A-13F. Quantification of G4 and P-G4 in plasma. FIGS. 13A-13C show liquid chromatography tandem mass spectrometry (LC-MS/MS) was used to generate a standard curve of G4 and P-G4 concentration in blood plasma. FIG. 13A shows LC-MS/MS parameters for in-source fragmentation and rigor of the calibration curve are summarized. FIG. 13B shows generation of the quantifying peptide. FIG. 13C shows a stepped higher-energy collisional dissociation approach effectively fragmented G4 in a single MS/MS spectrum. Fragment ions: b-ions; y-ions; glycan loss marked with arrows. Modified amino acids: bold faced. FIG. 13D shows plasma concentration of G4 and P-G4 over time after intravenous administration of a single weight-based (8 μmol/kg) dose. A non-occlusive thrombus was induced be electrolytic injury of the inferior vena cava and vessel thrombus weight measured 48 h after injury to determine treatment efficacy. FIG. 13E shows prophylactic administration of P-G6 (8 μmol/kg IV) and LMWH (6 mg/kg SC) demonstrated significant reduction in thrombus weight as compared to mice administered saline vehicle (n=8/group). (Data are mean±SEM; ***p<0.001 (ANOVA with Tukey's multiple comparison). Treatment with P-G6 does not affect hemostasis. FIG. 13F shows the effect of P-G6 on hemostasis was assessed using a tail vein bleeding assay. Mice were subject to intravenous administration of saline vehicle, P-G6 (8 μmol/kg) or LMWH (6 mg/kg) 5 min prior to transection of the lateral tail vein. LMWH demonstrated a significant increase in bleeding time as compared to mice receiving saline vehicle while no increase in bleeding time was observed after administration of P-G6. Data is represented as mean±SEM. Group comparisons were conducted using Welch's ANOVA with Dunnet's multiple comparison test. ***p<0.001.

FIGS. 14A-14C. FIG. 14A shows synthesis of aryl GSnP-4 intermediate 24. FIG. 14B shows synthesis of aryl GSnP-4 intermediate 27. FIG. 14C shows SPPS and chemoenzymatic synthesis of aryl GSnP-4.

FIG. 15 shows a synthesis of Compound 35.

FIG. 16 shows SPPS and chemical deprotection steps to yield aryl GSnP-4.

FIGS. 17A-17D show plasma concentrations of P-G4 measured using an ELISA assay over time after administration of a single subcutaneous dose of 16 μmol/kg (FIG. 17A), 8 μmol/kg (FIG. 17B), 4 μmol/kg (FIG. 17C), or 2 μmol/kg (FIG. 17D) in mice (n=4). FIG. 17E shows calculated pharmacokinetic parameters for subcutaneous delivery of P-G4.

FIG. 18A shows LC-MS measurement of plasma concentration of P-G4 after subcutaneous delivery at 4 μmol/kg. FIG. 18B shows ELISA measurement of plasma concentration of P-G4 after subcutaneous delivery at 4 μmol/kg.

FIGS. 19A-19B show dose-efficacy of P-G4 in C57BL/6 mice for inhibition of venous thrombosis in vivo. FIG. 19A shows thrombus weight excised from mice administered LMWH, saline (S), or P-G4. PEG-Gen 2 GSP was administered SC 4 hours prior and 24 hours after vena cava injury for all doses except at the 16 μmol/kg dose, which was administered as a single SC dose followed by vena cava injury 48 h later. Cohort 1r represents mice that received compound at 1 μmol/kg SC once daily (0, 24, 48, 72, 96 h). Vena cava injury was performed at 76 h. In all instances, the vena cava and thrombus were harvested 48 h after injury. H=Low molecular weight heparin (enoxaparin 6 mg/kg); ***p<0.001 with respect to saline vehicle (ANOVA with Tukey's multiple comparison). FIG. 19B shows plasma concentration of P-G4 at time of thrombus harvest in the two-dose protocol described for FIG. 19A.

### DETAILED DESCRIPTION OF INVENTION

There is a need to develop P-selectin inhibitors that exhibit both high affinity and specificity. Accordingly, in one aspect, provided herein is a glycopeptide, or a salt thereof, comprising the formula Y¹X¹Y²X²X³Y³X⁴X⁵X⁶Z¹X⁷W¹ (SEQ ID NO: 1), wherein:
W¹ is threonine or serine conjugated with a saccharide or polysaccharide via a linker L¹;
L¹ comprises a substituted or unsubstituted aryl group or a substituted or unsubstituted heteroaryl group;
X¹, X², X³, X⁴, X⁵, X⁶, and X⁷ are each individually and independently any amino acid;
Y¹, Y², and Y³ are each individually and independently tyrosine, phenylalanine, or phenylglycine, and wherein Y¹, Y², and Y³ are each independently unsubstituted or substituted with —SO₃H, —CH₂SO₃H, —CF₂SO₃H, —CO₂H, —CONH₂, —NHSO₂CH₃, —SO₂NH₂, or —CH₂PO₃H;
wherein at least one of Y¹, Y², and Y³ is substituted with —CH₂SO₃H; and
Z¹ is proline or hydroxyproline.

In certain embodiments, the saccharide or polysaccharide comprises one or more sugars, e.g., two or more, or three or more sugars. In certain embodiments, the polysaccharide comprises 2, 3, or 4 sugars. In certain embodiments, the sugars are selected from the group consisting of: 2-(acetylamino)-2-deoxy-galactose, galactose, 2-(acetylamino)-2-deoxy-glucose, fucose, and 5-acetamido-3,5-dideoxy-glycero-galacto-2-nonulosonic acid. In certain embodiments, the polysaccharide is sialyl Lewis X or sialyl Lewis A. In a particular embodiment, the polysaccharide is sialyl Lewis X. In another particular embodiment, the polysaccharide is sialyl Lewis X.

In certain embodiments, the polysaccharide comprises a radical S¹:

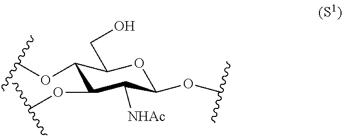

wherein L¹ is bonded to the anomeric oxygen of S¹.

In certain embodiments, the polysaccharide further comprises an α 1-3 bond between S¹ and a radical S²:

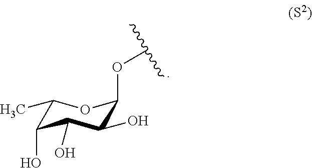

In certain embodiments, the polysaccharide further comprises a β 1-4 bond between S¹ and a radical S³:

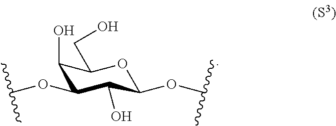

In certain embodiments, the polysaccharide further comprises a β 1-3 bond between S³ and a radical S⁴:

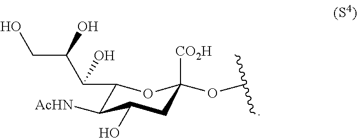

In certain embodiments, the polysaccharide is of the formula:

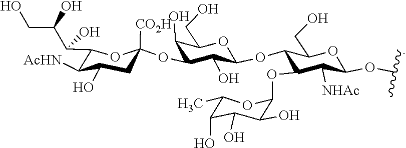

In certain embodiments, at least two of Y¹, Y², and Y³ are substituted with —CH₂SO₃H. In certain embodiments, each of Y¹, Y², and Y³ is substituted with —CH₂SO₃H. In certain particular embodiments, each of Y¹, Y², and Y³ is phenylglycine substituted with —CH₂SO₃H.

In a particular embodiment, W¹ is threonine. In another particular embodiment, W¹ is serine.

In certain embodiments, X¹, X³, X⁴, and X⁷ are each individually and independently E, D, N, or Q.

In certain embodiments, X², X⁵, and X⁶ are each individually and independently L, I, V, A or F.

In certain embodiments, the glycopeptide comprises: Y¹EY²LDY³DFLZ¹EW¹ (SEQ ID NO: 2), Y¹EY²LDY³DFLZ¹EW¹EP (SEQ ID NO: 3), Y¹EY²LDY³DFLZ¹EW¹EPL (SEQ ID NO: 4), EY¹EY²LDY³DFLZ¹EW¹ (SEQ ID NO: 5), EY¹EY²LDY³DFLZ¹EW¹E (SEQ ID NO: 6), EY¹EY²LDY³DFLZ¹EW¹EP (SEQ ID NO: 7), EY¹EY²LDY³DFLZ¹EW¹EPL (SEQ ID NO: 8), KEY¹EY²LDY³DFLZ¹EW¹ (SEQ ID NO: 9), KEY¹EY²LDY³DFLZ¹EW¹E (SEQ ID NO: 10), KEY¹EY²LDY³DFLZ¹EW¹EP (SEQ ID NO: 11), or KEY¹EY²LDY³DFLZ¹EW¹EPL (SEQ ID NO: 12).

In certain embodiments, L¹ comprises a substituted or unsubstituted aryl group. In certain embodiments, L¹ comprises a substituted or unsubstituted phenyl group. In certain embodiments, L¹ has the structure:

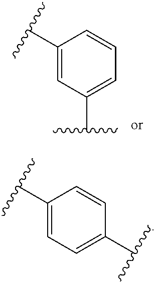 [SMILES: *c1cccc(*)c1 ; *c1ccc(*)cc1]

In certain embodiments, L¹ comprises a substituted or unsubstituted heteroaryl group. In certain embodiments, L¹ comprises a heteroaryl moiety.

In certain embodiments, L¹ comprises a triazole. In certain embodiments, L¹ comprises a 1,2,3-triazole. In certain particular embodiments, L¹ has the structure:

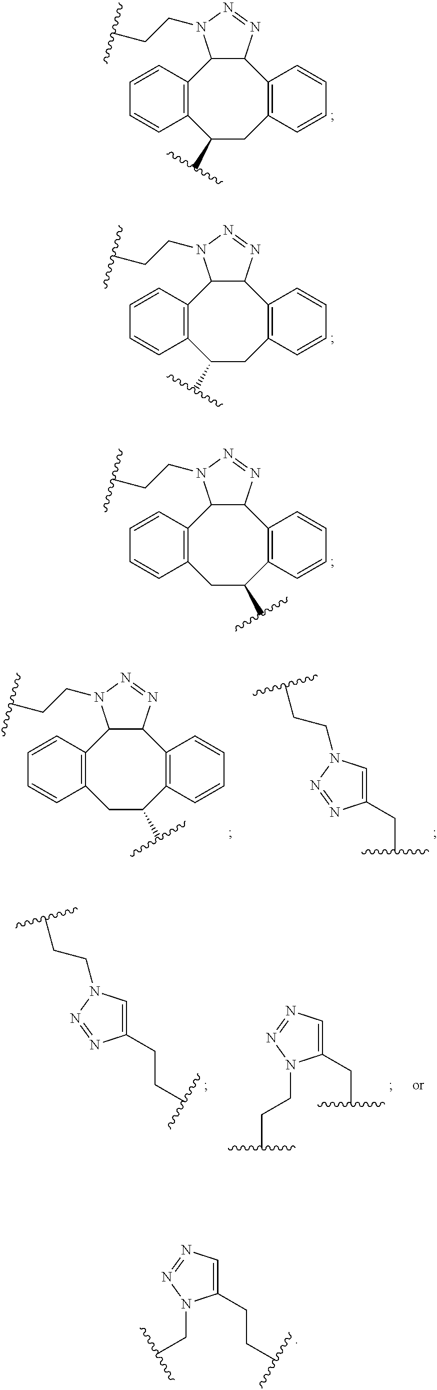

In certain embodiments, the glycopeptide, or salt thereof, comprises an N-terminal acetyl moiety. In certain embodiments, the glycopeptide, or salt thereof, comprises a substituted or unsubstituted aliphatic moiety, or a substituted or unsubstituted heteroaliphatic moiety. In certain embodiments, the aliphatic moiety is a substituted or unsubstituted alkyl moiety. In certain particular embodiments, the aliphatic moiety is a substituted or unsubstituted C₆-C₂₀ alkyl moiety. In certain embodiments, the aliphatic moiety is a fatty acid radical. In certain embodiments, the aliphatic moiety is palymitoyl.

In certain embodiments the heteroaliphatic moiety is polyethylene glycol (PEG). The PEG moiety may have 1-10000 repeat units. In certain embodiments, the PEG moiety has 1-10 repeat units, 10-100 repeat units, 100-1000 repeat units, 500-1000 repeat units, 800-1000 repeat units, 1000-3000 repeat units, 3000-6000 repeat units, 4000-8000 repeat units, or 6000-10000 repeat units.

In certain embodiments, the glycopeptide or salt thereof, comprises the formula Ac-KEY¹EY²LDY³DFLZ¹EW¹EPL (SEQ ID NO: 13), wherein:
W¹ is threonine conjugated with a polysaccharide via a linker L¹;
L¹ is

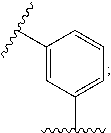 [SMILES: *c1cccc(*)c1]

and Y¹, Y², and Y³ are each phenylalanine substituted with —CH₂SO₃H.

In certain embodiments, the glycopeptide or salt thereof, comprises the formula Ac-KEY¹EY²LDY³DFLZ¹EW¹EPL (SEQ ID NO: 13), wherein:
W¹ is threonine conjugated with a polysaccharide via a linker L¹;
L¹ is

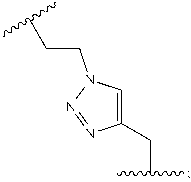 [SMILES: *CCn1cc(C*)nn1]

and Y¹, Y², and Y³ are each phenylalanine substituted with —CH₂SO₃H.

In certain embodiments, the glycopeptide has the structure of (G4):

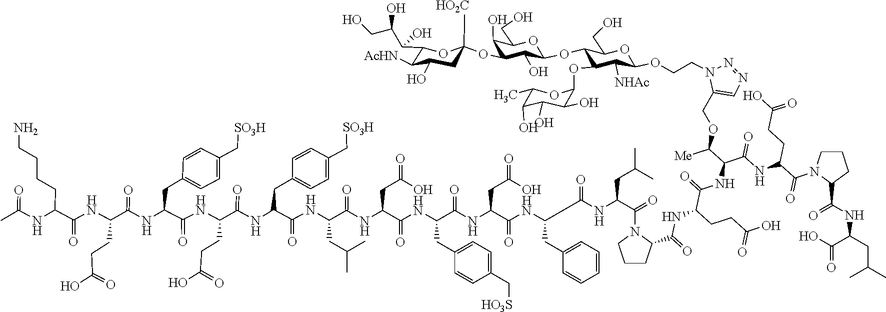

or a salt thereof.

In certain embodiments, the glycopeptide has the structure of (P-G4):

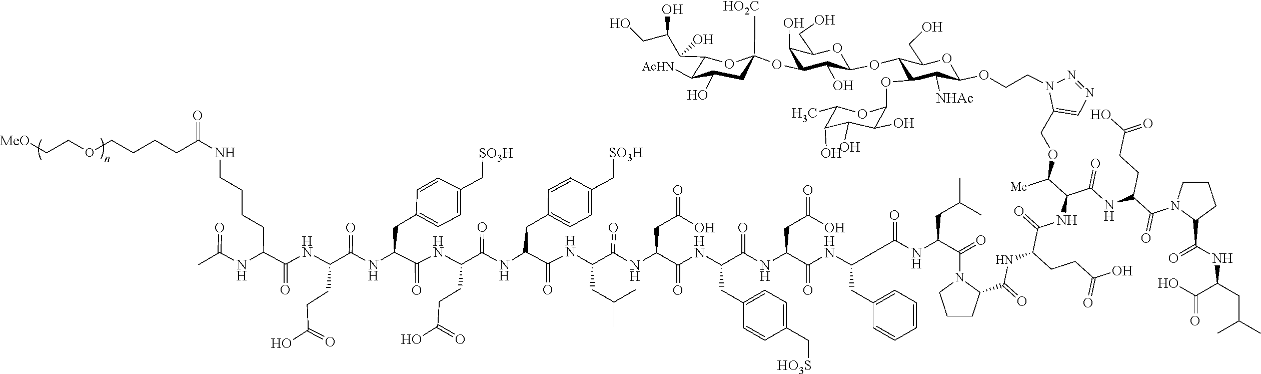

or a salt thereof; wherein n is 1-10,000.

In another aspect, provided herein is a pharmaceutical composition comprising a glycopeptide as described herein, or a salt thereof, and a pharmaceutically acceptable excipient.

In another aspect, provided herein is a pharmaceutical composition, optionally further comprising an additional therapeutic agent (e.g., an additional pharmaceutical agent as described herein). In certain embodiments, the pharmaceutical composition is formulated for intravenous delivery.

In another aspect, provided herein is a method, comprising administering to a subject a glycopeptide, or a salt thereof, as described herein, or a pharmaceutical composition as described herein.

In another aspect, provided herein is a method of inhibiting P-selectin binding to PSGL-1, comprising contacting the P-selectin with a glycopeptide, or salt thereof, as described herein. In certain embodiments, the method is in vitro. In certain embodiments, the method is in vivo.

In another aspect, provided herein is a method of treating or preventing cardiovascular disease, atherosclerosis, atherosclerotic lesions, thrombus formation, thromboembolism, stroke, or myocardial infarction in a subject in need thereof, comprising administering to the subject an effective amount of a glycopeptide, or a salt thereof, as described herein, or a pharmaceutical composition as described herein. In certain embodiments, the thromboembolism is venous thromboembolism (VTE). In certain embodiments, the VTE is cancer-associated.

In certain embodiments, the subject is at risk of, exhibiting symptoms of, or diagnosed with atherosclerosis, atherosclerotic lesions, thrombus formation, thromboembolism, stroke, or myocardial infarction. In certain embodiments, the subject has an increased risk of bleeding. In certain embodiments, the subject has a history of bleeding. In certain embodiments, the subject has a history of abnormal liver or kidney function, or has increased fall risk.

In another aspect, provided herein is a method of thromboprophylaxis, comprising administering to a subject diagnosed with cancer an effective amount of a glycopeptide as described herein, or a pharmaceutical composition as described herein.

In another aspect, provided herein is a method of treating or preventing allergy or lung disease in a subject in need thereof, comprising administering to the subject an effective amount of a glycopeptide as described herein, or a pharmaceutical composition as described herein. In certain embodiments, the subject is at risk of, exhibiting symptoms of, or diagnosed with asthma, bronchitis, emphysema, and COPD.

In some embodiments, the glycopeptide is administered subcutaneously. In some embodiments, the glycopeptide is administered intravenously.

In another aspect, provided herein is a method of making a glycopeptide, comprising reacting an oligosaccharide group comprising a first reactive moiety, with a peptide comprising a second reactive moiety, to obtain the glycopeptide, or a salt thereof;
wherein the first reactive moiety and the second reactive moiety react to form a heterocyclic moiety. In certain embodiments, the heterocyclic moiety comprises a triazole. In certain embodiments, the heterocyclic moiety comprises a dihydropridazine.

In some embodiments, the first reactive moiety comprises an azide, an alkyne, or a strained alkene. In a particular embodiment, the first reactive moiety comprises an azide. In another particular embodiment, the first reactive moiety comprises an alkyne. In another particular embodiment, the first reactive moiety comprises a strained alkene.

In certain embodiments, the oligosaccharide group has the structure:

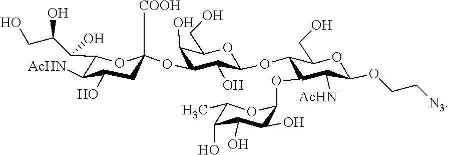

In some embodiments, the second reactive moiety comprises an azide, an alkyne, or a strained alkene. In a particular embodiment, the second reactive moiety comprises an azide.

In another particular embodiment, the second reactive moiety comprises an alkyne. In another particular embodiment, the second reactive moiety comprises a strained alkene.

In certain embodiments, the peptide has the structure:

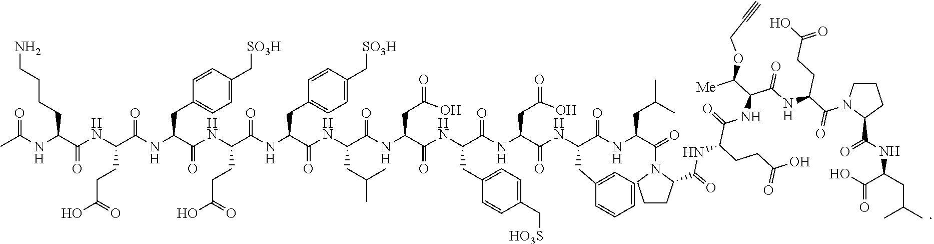 [SMILES: C#CCO[C@H](C)[C@H](NC(=O)[C@H](CCC(=O)O)NC(=O)[C@@H]1CCCN1C(=O)[C@H](CC(C)C)NC(=O)[C@H](Cc1ccccc1)NC(=O)[C@H](CC(=O)O)NC(=O)[C@H](Cc1ccc(CS(=O)(=O)O)cc1)NC(=O)[C@H](CC(=O)O)NC(=O)[C@H](CC(C)C)NC(=O)[C@H](Cc1ccc(CS(=O)(=O)O)cc1)NC(=O)[C@H](CCC(=O)O)NC(=O)[C@H](Cc1ccc(CS(=O)(=O)O)cc1)NC(=O)[C@H](CCC(=O)O)NC(=O)[C@H](CCCCN)NC(C)=O)C(=O)N[C@@H](CCC(=O)O)C(=O)N1CCC[C@H]1C(=O)N[C@@H](CC(C)C)C(=O)O]

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Unless otherwise provided, formulae and structures depicted herein include compounds that do not include isotopically enriched atoms, and also include compounds that include isotopically enriched atoms. For example, compounds having the present structures except for the replacement of hydrogen by deuterium or tritium, replacement of 19F with 18F, or the replacement of a carbon by a 13C- or 14C-enriched carbon are within the scope of the disclosure. Such compounds are useful, for example, as analytical tools or probes in biological assays. [0004] The term “isotopes” refers to variants of a particular chemical element such that, while all isotopes of a given element share the same number of protons in each atom of the element, those isotopes differ in the number of neutrons.

When a range of values (“range”) is listed, it encompasses each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

The term “heteroaryl” refers to a radical of a 5-14 membered monocyclic or polycyclic (e.g., bicyclic, tricyclic) 4n+2 aromatic ring system (e.g., having 6, 10, or 14 π electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not limited in any manner by the exemplary substituents described herein.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR—, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};
wherein:
   each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{cc})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀alkyl, heteroC₁₋₁₀alkenyl, heteroC₁₋₁₀alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form ═O or ═S; wherein X⁻ is a counterion;
   each instance of R^{ee} is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{f} is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two R^{f} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃-C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R^{gg} substituents can be joined to form ═O or ═S; and
   each X⁻ is a counterion.

In certain embodiments, each carbon atom substituent is independently halogen, substituted (e.g., substituted with one or more halogen) or unsubstituted C₁₋₆ alkyl, ═O, —OR^{aa}, —SR—, —N(R^{bb})₂, —CN, —SCN, —NO₂, —C(═O)R^{aa}, —CO₂R—, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂.

The terms “polysaccharide” and “oligosaccharide” are used interchangeably herein.

A “counterion” or “anionic counterion” is a negatively charged group associated with a positively charged group in order to maintain electronic neutrality. An anionic counterion may be monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F₅)₄, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

These and other exemplary substituents are described in more detail in the Detailed Description, Examples, and Claims. The invention is not limited in any manner by the above exemplary listing of substituents.

The following definitions are more general terms used throughout the present application.

As used herein, the term “salt” refers to any and all salts, and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of this invention include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human comprises about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for inhibition of PSGL-1/P-selectin binding. In certain embodiments, a therapeutically effective amount is an amount sufficient for treating a disease or condition recited herein (e.g., VTE).

A “prophylactically effective amount” of a compound described herein is an amount sufficient to prevent a condition, or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for inhibition of PSGL-1/P-selectin binding. In certain embodiments, a prophylactically effective amount is an amount sufficient for treating a disease or condition recited herein (e.g., VTE).

The term “prevent,” “preventing,” or “prevention” refers to a prophylactic treatment of a subject who is not and was not with a disease but is at risk of developing the disease or who was with a disease, is not with the disease, but is at risk of regression of the disease. In certain embodiments, the subject is at a higher risk of developing the disease or at a higher risk of regression of the disease than an average healthy member of a population.

As used herein the term “inhibit” or “inhibition” in the context of enzymes, for example, in the context of P-selectin, refers to a reduction in the activity of the enzyme. In some embodiments, the term refers to a reduction of the level of enzyme activity, e.g., P-selectin activity, to a level that is statistically significantly lower than an initial level, which may, for example, be a baseline level of enzyme activity. In some embodiments, the term refers to a reduction of the level of enzyme activity, e.g., P-selectin activity, to a level that is less than 75%, less than 50%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of enzyme activity.

### Pharmaceutical Compositions, Kits, and Administration

The present disclosure provides pharmaceutical compositions comprising a glycopeptide as disclosed herein, or a salt (e.g., a pharmaceutically acceptable salt) thereof, and optionally a pharmaceutically acceptable excipient. In certain embodiments, the pharmaceutical composition described herein comprises a glycopeptide as disclosed herein, or a pharmaceutically acceptable salt thereof, and a pharmaceutically acceptable excipient.

In certain embodiments, the compound (i.e., glycopeptide) described herein is provided in an effective amount in the pharmaceutical composition. In certain embodiments, the effective amount is a therapeutically effective amount. In certain embodiments, the effective amount is a prophylactically effective amount. In certain embodiments, the effective amount is an amount effective for inhibiting the activity (e.g., aberrant activity, such as increased activity) of an enzyme (e.g., P-selectin) in a subject or cell.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the cell is present in vitro. In certain embodiments, the cell is present in vivo.

Pharmaceutical compositions described herein can be prepared by any method known in the art of pharmaceutics. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject. In some embodiments, the glycopeptide is administered subcutaneously. In some embodiments, the glycopeptide is administered intravenously.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose or single subcutaneous dose) or multiple doses (e.g., multiple oral doses or multiple subcutaneous doses). In certain embodiments, a single dose is administered to a subject or applied to a tissue or cell. In certain embodiments, a single dose is administered to a subject or applied to a tissue or cell prior to injury.

In certain embodiments, when multiple doses are administered to a subject or applied to a tissue or cell, any two doses of the multiple doses include different or substantially the same amounts of a compound described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is one dose per day prior to injury. In certain embodiments, the frequency of administering the multiple doses to the subject or applying the multiple doses to the tissue or cell is one dose per day prior to injury and once subsequent to injury. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, the duration between the first dose and a subsequent dose of the multiple doses is about 20 to about 32 hours. In certain embodiments, the duration between the first dose and a subsequent dose of the multiple doses is about 24 to about 28 hours. In certain embodiments, the duration between the first dose and a subsequent dose of the multiple doses is about 24 hours. In certain embodiments, the duration between the first dose and a subsequent dose of the multiple doses is about 28 hours.

In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, the duration between the first dose and last dose of the multiple doses is about 24 to 30 hours. In certain embodiments, the duration between the first dose and last dose of the multiple doses is about 28 hours. In certain embodiments, the duration between the first dose and last dose of the multiple doses is about 1 to 5 days. In certain embodiments, the duration between the first dose and last dose of the multiple doses is about 1 day. In certain embodiments, the duration between the first dose and last dose of the multiple doses is about 5 days.

In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

In certain embodiments, the pharmaceutical composition comprises about 0.1 μmol/kg, about 0.5 μmol/kg, about 1 μmol/kg, about 2 μmol/kg, about 3 μmol/kg, about 4 μmol/kg, about 5 μmol/kg, about 6 μmol/kg, about 7 μmol/kg, about 8 μmol/kg, about 9 μmol/kg, about 10 μmol/kg, about 11 μmol/kg, about 12 μmol/kg, about 13 μmol/kg, about 14 μmol/kg, about 15 μmol/kg, about 16 μmol/kg, about 17 μmol/kg, about 18 μmol/kg, about 19 μmol/kg, about 20 μmol/kg, about 25 μmol/kg, about 50 μmol/kg, or about 100 μmol/kg of a compound described herein. In certain embodiments, the pharmaceutical composition comprises about 0.1 μmol/kg to about 100 μmol/kg of a compound described herein. In certain embodiments, the pharmaceutical composition comprises about 0.1 μmol/kg to about 50 μmol/kg of a compound described herein. In certain embodiments, the pharmaceutical composition comprises about 0.1 μmol/kg to about 25 μmol/kg of a compound described herein. In some embodiments, the pharmaceutical composition comprises about 0.1 μmol/kg to about 20 μmol/kg of a compound described herein. In certain embodiments, the pharmaceutical composition comprises about 0.1 μmol/kg to about 15 μmol/kg of a compound described herein. In some embodiments, the pharmaceutical composition comprises about 0.1 μmol/kg to about 10 μmol/kg of a compound described herein. In certain embodiments, the pharmaceutical composition comprises about 0.1 μmol/kg to about 5 μmol/kg of a compound described herein. In some embodiments, the pharmaceutical composition comprises about 1 μmol/kg to about 100 μmol/kg of a compound described herein. In some embodiments, the pharmaceutical composition comprises about 1 μmol/kg to about 50 μmol/kg of a compound described herein. In some embodiments, the pharmaceutical composition comprises about 1 μmol/kg to about 25 μmol/kg of a compound described herein. In certain embodiments, the pharmaceutical composition comprises about 1 μmol/kg to about 20 μmol/kg of a compound described herein. In some embodiments, the pharmaceutical composition comprises about 1 μmol/kg to about 15 μmol/kg of a compound described herein. In certain embodiments, the pharmaceutical composition comprises about 1 μmol/kg to about 10 μmol/kg of a compound described herein. In some embodiments, the pharmaceutical composition comprises about 1 μmol/kg to about 5 μmol/kg of a compound described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof, and/or in inhibiting the activity of a protein kinase in a subject or cell), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compound and the additional pharmaceutical agent, but not both. In some embodiments, the additional pharmaceutical agent achieves a desired effect for the same disorder. In some embodiments, the additional pharmaceutical agent achieves different effects.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease, hematological disease, neurological disease, painful condition, psychiatric disorder, or metabolic disorder). Each additional pharmaceutical agent may be administered at a dose and/or on a time schedule determined for that pharmaceutical agent. The additional pharmaceutical agents may also be administered together with each other and/or with the compound or composition described herein in a single dose or composition or administered separately in different doses or compositions. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, steroidal or non-steroidal anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, anesthetics, anti-coagulants, inhibitors of an enzyme, steroidal agents, steroidal or antihistamine, antigens, vaccines, antibodies, decongestant, sedatives, opioids, analgesics, anti-pyretics, hormones, and prostaglandins. In certain embodiments, the additional pharmaceutical agent is an anti-proliferative agent. In certain embodiments, the additional pharmaceutical agent is an anti-cancer agent. In certain embodiments, the additional pharmaceutical agent is an anti-viral agent. In certain embodiments, the additional pharmaceutical agent is an binder or inhibitor of a protein kinase. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine protein kinase inhibitors), modulators of protein stability (e.g., proteasome inhibitors), Hsp90 inhibitors, glucocorticoids, all-trans retinoic acids, and other agents that promote differentiation. In certain embodiments, the compounds described herein or pharmaceutical compositions can be administered in combination with an anti-cancer therapy including, but not limited to, surgery, radiation therapy, transplantation (e.g., stem cell transplantation, bone marrow transplantation), immunotherapy, and chemotherapy. Additional pharmaceutical agents include small organic molecules such as drug compounds (e.g., compounds approved by the US Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins and cells.

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating a disease (e.g., proliferative disease, hematological disease, neurological disease, painful condition, psychiatric disorder, or metabolic disorder) in a subject in need thereof. In certain embodiments, the kits are useful for preventing a disease (e.g., proliferative disease, hematological disease, neurological disease, painful condition, psychiatric disorder, or metabolic disorder) in a subject in need thereof. In certain embodiments, the kits are useful for reducing the risk of developing a disease (e.g., proliferative disease, hematological disease, neurological disease, painful condition, psychiatric disorder, or metabolic disorder) in a subject in need thereof. In certain embodiments, the kits are useful for inhibiting the activity (e.g., aberrant activity, such as increased activity) of a protein kinase in a subject or cell.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., proliferative disease, hematological disease, neurological disease, painful condition, psychiatric disorder, or metabolic disorder) in a subject in need thereof. In certain embodiments, the kits and instructions provide for preventing a disease (e.g., proliferative disease, hematological disease, neurological disease, painful condition, psychiatric disorder, or metabolic disorder) in a subject in need thereof. In certain embodiments, the kits and instructions provide for reducing the risk of developing a disease (e.g., proliferative disease, hematological disease, neurological disease, painful condition, psychiatric disorder, or metabolic disorder) in a subject in need thereof. In certain embodiments, the kits and instructions provide for inhibiting the activity (e.g., aberrant activity, such as increased activity) of a protein kinase in a subject or cell. A kit described herein may include one or more additional pharmaceutical agents described herein as a separate composition.

### Diseases and Disorders

The term “cancer” refers to a class of diseases characterized by the development of abnormal cells that proliferate uncontrollably and have the ability to infiltrate and destroy normal body tissues. See e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “cardiovascular disease” refers to diseases and disorders of the heart and circulatory system. Exemplary cardiovascular diseases, including cholesterol- or lipid-related disorders, include, but are not limited to acute coronary syndrome, angina, arrhythmia, arteriosclerosis, atherosclerosis, carotid atherosclerosis, cerebrovascular disease, cerebral infarction, congestive heart failure, congenital heart disease, coronary heart disease, coronary artery disease, coronary plaque stabilization, dyslipidemias, dyslipoproteinemias, endothelium dysfunctions, familial hypercholeasterolemia, familial combined hyperlipidemia, hypoalphalipoproteinemia, hypertriglyceridemia, hyperbetalipoproteinemia, hypercholesterolemia, hypertension, hyperlipidemia, intermittent claudication, ischemia, ischemia reperfusion injury, ischemic heart diseases, cardiac ischemia, metabolic syndrome, multi-infarct dementia, myocardial infarction, obesity, peripheral vascular disease, reperfusion injury, restenosis, renal artery atherosclerosis, rheumatic heart disease, stroke, thrombotic disorder, transitory ischemic attacks, and lipoprotein abnormalities associated with Alzheimer's disease, obesity, diabetes mellitus, syndrome X, impotence, multiple sclerosis, Parkinson's diseases inflammatory diseases, lesions, thrombus formation, and thromboembolism.

### INCORPORATION BY REFERENCE

The present application refers to various issued patent, published patent applications, scientific journal articles, and other publications, all of which are incorporated herein by reference. The details of one or more embodiments of the invention are set forth herein. Other features, objects, and advantages of the invention will be apparent from the Detailed Description, the Figures, the Examples, and the Claims.

### EXAMPLES

### Example 1

The P-selectin/PSGL-1 pathway plays a critical role in the initiation and propagation of venous thrombosis by facilitating the accumulation of leukocytes and platelets within the growing thrombus. To develop novel inhibitors of this pathway, molecular dynamics simulations were used to examine a series of structural analogues of the N-terminal domain of PSGL-1 and their interactions were compared with P-selectin against the natural ligand. A specific and unique analogue, G4, was identified and a total chemical synthesis scheme was developed. The candidate P-selectin antagonist was synthesized and validated in a microarray binding assay, and surface plasma resonance (SPR) analysis demonstrated that the analogue exhibits nanomolar inhibition of PSGL-1/P-selectin binding. A PEGylated version of this analogue, PEG40-G4 (P-G4), is a highly potent P-selectin inhibitor with a favorable pharmacokinetic profile. P-G4 blocks P-selectin interactions with human and mouse neutrophils in a dose-dependent manner and inhibits human and mouse platelet-monocyte and platelet-neutrophil aggregation. P-G4 reduces thrombus formation in a murine model of venous thrombosis, but without disruption of hemostasis. P-G4 potently inhibits the P-selectin/PSGL-1 pathway and represents a promising drug candidate for the prevention of venous thrombosis, particularly for those patients who are at increased bleeding risk.

Second only to the underlying malignancy, venous thromboembolism (VTE) is the leading cause of death in cancer patients. VTE occurs in approximately 20% of all cancer patients (1) with a 4- to 7-fold increased risk compared to those without cancer and a 20-fold increased risk for those patients with metastatic disease (2, 3). Significantly, cancer-associated VTE accounts for 20-30% of all thrombotic events (4). VTE can delay the initiation of cancer treatment, and all cancer therapies, including chemotherapy, targeted therapy, anti-angiogenic therapy, hormonal therapy, radiation therapy, and surgery may further increase VTE risk (1). Significantly, VTE affects nearly one-third of all cancer patients receiving newer immunotherapy agents and has been associated with worse survival (5).

Current guidelines recommend primary thromboprophylaxis with anticoagulation for hospitalized patients with an active cancer. However, meta-analyses and randomized controlled trials that have evaluated various anticoagulation regimens demonstrate that conventional agents provide inadequate protection from cancer-associated VTE and are associated with an increased risk of major bleeding (6-9). Despite such high risks, it is anticipated that newer guidelines will continue to recommend extended prophylaxis for high-risk ambulatory patients, especially for those with pancreatic and gastric adenocarcinoma (10, 11) or with metastatic disease (2). Once VTE occurs, cancer patients have a three- to four-fold higher rate of VTE recurrence compared to patients without cancer (6, 12). Most guidelines for secondary thromboprophylaxis recommend continuing therapy for at least 6 months, or longer if the cancer is still present or the patient is continuing to receive chemotherapy or radiation therapy (13, 14). Nevertheless, even in the absence of active cancer therapy, recent studies support extended thromboprophylaxis beyond the initial 6-month treatment period (4, 15, 16). Current anticoagulation regimens provide incomplete protection from recurrent VTE and are associated with a significant bleeding risk. Low molecular weight heparin (LMWH) reduces the risk of recurrent cancer-associated VTE as compared to warfarin, but increases the likelihood of major bleeding (17, 18). Two large multicenter randomized controlled trials (RCTs) in cancer patients, Hokusai VTE Cancer (16) and SELECT-D (19), recently demonstrated that direct oral anticoagulants (DOACs), such as direct factor Xa inhibitors are non-inferior to LMWH, but result in an increased incidence of major bleeding events (20). Each of these trials also excluded high risk subpopulations, including patients with poor ECOG (Eastern Cooperative Oncology Group) performance status, brain metastases, or thrombocytopenia. A recent analysis of 26,894 patients (21) demonstrated that DOAC associated bleeding risk per patient-year was very high for those with upper GI cancers (8.6%), followed by lung cancer (6.0%), colorectal cancer (4.5%), prostate cancer (4.0%), hematological cancer (3.5%), and breast cancer (2.9%). Therefore, there is an urgent need to develop novel therapeutics without bleeding risk for preventing primary and recurrent VTE in cancer patients.

More effective therapies are required to reduce the risk of primary and recurrent VTE among cancer patients, with the standard of care LMWH and conventional anticoagulants (DOACs) all limited by incomplete protection and a significantly increased risk of major bleeding (6, 16, 19, 21, 22). Newer anti-thrombotic agents have been proposed to potentially display a reduced risk of major bleeding, but thus far have not demonstrated an improved safety profile (23, 24). Because cancer patients are not only at an increased risk for VTE, but also at greater risk of bleeding, the use of anticoagulants is entirely excluded for those patients with a prior history of bleeding, abnormal liver or kidney function, or at increased fall risk due to advanced age, frailty, unsteadiness or deconditioning following surgery, chemotherapy or radiation therapy. Therefore, effectively managing cancer-associated VTE requires novel therapies that do not interfere with the coagulation process and carry an attendant risk of bleeding.

Extensive intravascular fibrin formation has led to the presumption that the coagulation system plays a dominant role in venous thrombosis. However, recent studies demonstrate that leukocytes are critical for both the initiation and propagation of VTE. Specifically, numerous studies have suggested that monocyte- and neutrophil-derived tissue factor (TF) is a decisive initial trigger for massive fibrin deposition (25-27). Moreover, continued neutrophil accumulation, as well as neutrophil-platelet interactions lead to the release of procoagulant neutrophil extracellular traps (NETs), which promotes thrombus propagation (28-32). P-selectin glycoprotein ligand-1 (PSGL-1) is a glycoprotein that is expressed on the surface of all leukocytes and supports leukocyte recruitment as a component of a broad range of inflammatory responses. PSGL-1 is a ligand for E-, P- and L-selectins, but binds with highest affinity to P-selectin, which is expressed on the surfaces of activated platelets and endothelial cells. Ligation of P-selectin on endothelial cells by PSGL-1 on leukocytes constitutes the initial ‘capture and rolling’ step in the leukocyte-endothelial cell adhesion cascade. Likewise, the interaction of leukocyte PSGL-1 with P-selectin on activated platelets leads to leukocyte-platelet aggregates that promote the adhesion and infiltration of inflammatory cells and the release of circulating pro-coagulant microparticles (33).

Substantial evidence supports the view that PSGL-1/P-selectin interactions are critical to the pathogenesis of cancer-associated VTE. Many cancers secrete abnormally glycosylated proteins that can interact with platelet P-selectin, leading to the activation and aggregation of platelets (34) and the formation of platelet-leukocyte aggregates, which are crucial for the generation of VTE (35). High plasma levels of P-selectin are an indicator of risk for first and recurrent VTE (36-38) and represents a validated biomarker of patients at increased risk of cancer-associated VTE (39, 40). Both P-selectin- and PSGL-1-deficient mice display a reduction in venous thrombosis and circulating pro-coagulant microparticles (41-43).

The synthesis of a novel glycopeptide, GSnP-6, was previously reported as the first synthetic high affinity (Kd 22 nM) and specific P-selectin antagonist for human therapy (44). GSnP-6 blocks P-selectin binding to human and murine leukocytes in a dose-dependent manner, as well as leukocyte rolling and arrest. In addition, platelet-neutrophil and platelet-monocyte aggregation were inhibited in human blood, as were early thromboinflammatory events in vivo, as characterized by platelet adhesion to leukocytes and platelet microaggregate formation. Notably, GSnP-6 does not inhibit platelet activation or the coagulation pathway. Significantly, in a recent study, it was reported that GSnP-6 is a highly potent in vivo inhibitor of venous thrombosis but does not prolong bleeding time (45).

In an effort to better understand the molecular mechanisms responsible for the binding interactions of GSnP-6 and PSGL-1, molecular dynamics (MD) simulations were conducted to reproduce the salient structural and energetic features of the interactions of the N-terminal domain of PSGL-1 with P-selectin (44). These simulations predicted that GSnP-6 and PSGL-1 behave comparably, consistent with observed affinity data. Importantly, the development of a computational model established a useful framework to further guide the design of structurally simpler analogues that display comparable or higher selectin binding affinity. For example, theoretical per-residue interaction energies for the carbohydrate moieties Neu5Ac, Core-2 Gal, GlcNAc, and Fuc in the native ligand and in GSnP-6 are substantially greater than the interaction energies for GalNAc and Gal. This observation suggested that GalNAc and Gal are non-critical monosaccharides that could be replaced with structural analogues to simplify chemical synthesis. It bears emphasis that replacement of these sugars with a highly flexible spacer, such as a poly(ethylene glycol) linker, would likely afford a compound with substantially reduced binding affinity due to the high entropic cost of achieving optimal conformational alignment within the P-selectin binding pocket. This disclosure reports the use of MD simulations and interaction energy calculations to examine a series of simplified structural analogues of GSnP-6 to evaluate the similarity of new putative inhibitors to the natural ligand. Computational simulations were also used to determine the conformational preferences of each new analogue in order to compare their 3D structures to the known structure of PSGL-1. Through these efforts, a simplified analogue with reduced complexity in chemical synthesis was identified, and it was validated as a nanomolar inhibitor of PSGL-1/P-selectin binding interactions. A PEGylated version of this compound, with an extended circulating half-life, effectively inhibited P-selectin binding to PSGL-1, reduced human and murine platelet-leukocyte aggregation, and inhibited venous thrombosis in a pre-clinical model of VTE without increasing bleeding time.

### Methods:

### Molecular Modeling Studies

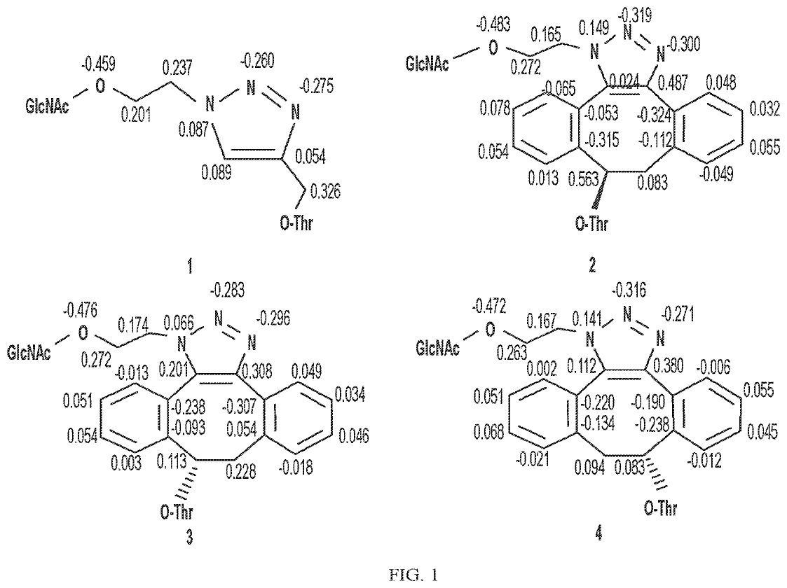

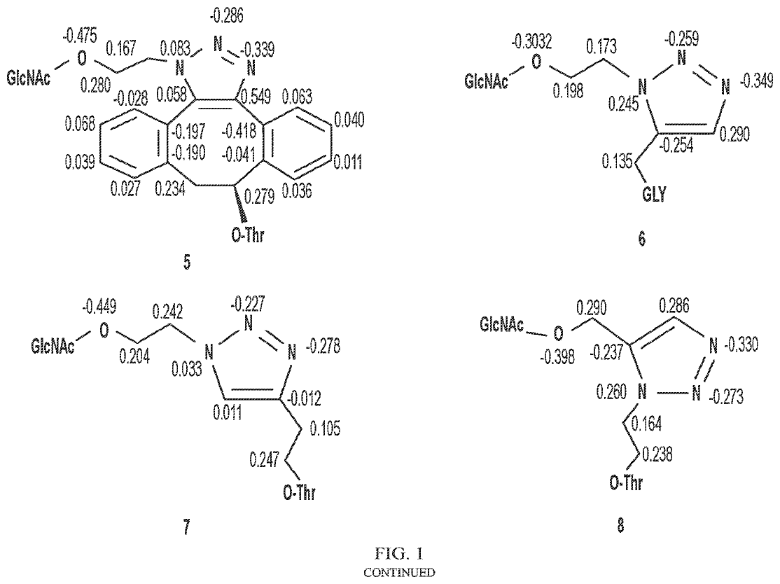

Force field parameters for the oligosaccharide, as well as the sulfate (SO₃) moiety in the tyrosine sulfonate (YCS) residues were obtained from the GLYCAM06 (version j) (46) parameter set, while those for the protein were taken from AMBER15 (ff99sb) (47). Valence parameters and partial atomic charges for the YCS residues in the peptide have been previously reported (44). The initial coordinates for the P-selectin/GSnP-6 complex were generated by replacing the phenolic oxygen atom in the tyrosine sulfate residues in the P-selectin/PSGL-1 co-crystal structure (PDB ID: 1G1S) with a methylene group (44). The initial coordinates for the various linkers between the peptide and oligosaccharide were built using GaussView Version 3 (Gaussian, Inc). Molecular electrostatic potentials (ESPs) were computed at the HF/6-31G*//HF-6-31G* level with the Gaussian09 software package (48) and restrained electrostatic potential (RESP) charge fitting was performed using the RESP procedure with a restraint weight of 0.01 (FIG. 1). During charge fitting, the partial atomic charges on the amino acid backbone charges were constrained to standard values employed in ff99sb. MD simulations were initiated with RESP charge sets computed for single conformations of each residue.

The initial coordinates for complexes of P-selectin and various GSnP-6 analogues were obtained from the P-selectin/PSGL-1 crystal structure (PDB ID: 1G1S). The initial coordinates for GSnP-6 analogues were generated by replacing the GalNAc and Gal residues in GSnP-6 with linker molecules, for which force field valence parameters were taken from the general AMBER force field (GAFF), GLYCAM06, or ff99SB parameter sets, as appropriate (Supplementary Table 2). Ligands were solvated with 8 Å TIP3P water buffer in a cubic box (49). Energy minimization was performed in two stages. First, the solvent was minimized under nVT conditions with cartesian restraints applied to the solute with a restraint weight of 100 kcal mol⁻¹ Å⁻². In the second stage, restraints were only applied on the peptide backbone. Prior to MD simulation, systems were subject to energy minimizations with 500 steps steepest descent followed by 24,500 steps of conjugate-gradient minimization. Subsequently, heating was performed over 50 ps (nVT) to 300° K with a weak restraint (10 kcal mol⁻¹ Å⁻²) on peptide backbone atoms. Systems were then equilibrated at 300° K for 0.5 ns using nPT conditions with the Berendsen thermostat prior to production MD (50), under the same conditions (covalent bonds involving hydrogen atoms constrained using SHAKE) (51). Production MD simulations were for 200 ns performed with the GPU implementation of PMEMD from AMBER15 (52). In all MD simulations, a non-bonded cut-off of 8 Å was applied to van der Waals interactions, with long-range electrostatics treated with the particle mesh Ewald approximation, and mixed 1-4 non-bonded scale factors applied, as recommended for systems containing both carbohydrates and proteins (SCEE=SCNB=1.0 for the oligosaccharide and SCEE=1.2 and SCNB=2.0 for the protein) (46). Each MD simulation was performed in triplicate to compute statistical properties. For ligand configurational entropy calculations, only the oligosaccharide and the O-linked peptide residue were considered.

Molecular Mechanics with Generalized Born Surface Approximation (MM-GBSA) Interaction Energies and Entropy Calculations.

MM-GBSA energy calculations were performed using the single-trajectory method (53) with the MMPBSA.py.MPI module in the AMBER15 software package (47), in which the GB₁^{OBC} model (α=0.8, β=0.0, γ=2.909125, igb=2) with an internal dielectric constant value (εᵢₙₜ) of 4.0 was employed. MM-GBSA energy calculations were performed on 5,000-snapshots evenly extracted from each of the three replicate MD trajectories for each system. Quasi-harmonic (QH) entropies (ΔS_{RTV}) were calculated using the cpptraj module in AMBERTOOLS (54, 55) on ligands bearing oligosaccharide, linker moiety, and O-linked amino acid. QH entropy values were fit linearly as a function of inverse simulation time to obtain the infinite sampling limit of the QH entropy at the Y-intercept of this plot (55). Conformational entropies associated with changes in the glycosidic torsion angle distributions, which occur upon binding were computed using the Karplus-Kushick approach (56). Average entropy values were obtained from the three individual trajectories.

### General

All reagents were purchased from commercial sources and used as received, unless otherwise indicated. All solvents were dried and distilled by standard protocols. All reactions were performed under an inert atmosphere of argon, unless otherwise noted. For peptide synthesis, a standard fluorenylmethyloxy carbonyl (Fmoc) solid phase protocol was employed. Fmoc-Leu Novasyn TGA resin (resin loading: 0.19 mmol g⁻¹), N,N-dimethylformamide (DMF, 227056), N,N′-diisopropylethylamine (DIPEA, 387649), and coupling reagent hexafluorophosphate benzotriazole tetramethyl uranium (HBTU, 12804) were purchased from Sigma-Aldrich. Fmoc-Phe(SO₃H)—OH was purchased from RSP Amino Acids LLC and other Fmoc amino acids purchased from Sigma-Aldrich. For surface plasma resonance (SPR) Biacore analysis, EZ-link biotin N-hydroxysuccinimide ester (NHS-biotin; 20217) was purchased from Thermo Scientific. For glycan synthesis, all organic phase extractions were dried over sodium sulfate and concentrated under vacuum. Chromatographic purifications were carried out over silica gel (60, 0.060-0.2 mm, 70-230 mesh). Analytical thin-layer chromatography was performed using pre-coated glass backed plates and visualized by UV irradiation (254 nm) or stained with 25% H₂SO₄ in ethanol or ceric ammonium molybdate solution. Specific rotations were measured using a digital polarimeter (Azzota) in the indicated solvent. The PEGylation reagent, 40 kDa methoxy poly(ethylene glycol)-succinimidyl valerate (mPEG-SVA) was purchased from Advanced Biochemicals (MOP2506, PDI 1.06). Reverse phase high performance liquid chromatography (RP-HPLC) was performed using the Waters 2767 Gradient Purification System with a Waters 2489 UV/Vis detection module and a Waters 2545 Binary Gradient Module, equipped with either a C18 100 Å preparative (250×30 mm, Phenomenex) or analytical (50×4.6 mm, Phenomenex) column. A Bruker Ultraflex II matrix-assisted laser desorption/ionization time of flight (MALDI-TOF) mass spectrometer was used to analyze samples co-crystallized with the Super-DHB (2,3-dihydroxylbenzoic acid) matrix. ¹H, ¹³C, P and 2D nuclear magnetic resonance (NMR) spectra were recorded using Varian MR 400 MHz and Bruker Avance II 600 MHz spectrometers. Chemical shifts were reported in δ (ppm) relative units to residual solvent peaks for CDCl₃ (7.26 ppm for ¹H and 77.0 ppm for ¹³C) and CD₃OD-d₄ (3.35 ppm and 4.78 ppm for ¹H and 49.3 ppm for ¹³C). Splitting patterns were assigned as s (singlet), d (doublet), t (triplet), q (quartet), quint (quintet), multiplet (m), dd (doublet of doublets), and td (triplet of doublets). Murine and human protocols were approved by the Institutional Animal Care and Use Committee and Institutional Review Board of Beth Israel Deaconess Medical Center.

All experimental protocols for compound synthesis, characterization data sets for all compounds, including NMR, HPLC and MALDI profiles, as well as details of computational and biological studies are provided in Supplementary Information.

Synthesis of 2-Azidoethyl Sialyl Lewis^{X} Detailed synthesis and characterization of 2-azidoethyl sialyl Lewis^{X} is described in Supplementary Information.

### Synthesis of Propargyl Sulfonated Peptide.

Fmoc protected propargyl threonine was synthesized according to a previous protocol (57). Coupling method A: A suspension of Fmoc protected amino acid (5.0 equiv., 0.5 M), HBTU (5.0 equiv., 0.5 M), and DIPEA (12.5 equiv., 0.5 M) in dimethylformamide (DMF) was transferred to the reaction vessel containing the resin. The resulting suspension was shaken for 1 h at 25° C. The reaction vessel was then drained and thoroughly washed with DMF. Coupling method B: A suspension of Fmoc protected amino acid (5.0 equiv., 0.5 M), HBTU (5.0 equiv., 0.5 M), and DIPEA (12.5 equiv., 0.5 M) in DMF was transferred to the reaction vessel containing the resin. The resulting suspension was shaken for 2 h at 25° C. The reaction vessel was then drained and thoroughly washed with DMF. Deprotection method: The deprotection step was carried out using excess 20% piperidine (400 μL/10 μmol) for 8 min at 25° C. The reaction vessel was drained and the deprotection step was repeated. Upon completion, the reaction vessel was washed with DMF and drained. The linear synthesis was carried out using Fmoc-Leu TGA NovaSyn resin solid support (0.19 mmol g⁻¹) prepared in a polypropylene centrifuge filter tube (0.22 μm, Corning International) equipped with a plastic cap. Amino acids L6, D7, D9, F10, L11, P12, E13, T14, E15, and P16 were coupled using coupling method A; amino acids K1, E2, Fs3, E4, Fs5, and Fs8 were coupled using coupling method 2. All couplings and deprotections were monitored using the Kaiser Test and MALDI-TOF HRMS. Upon completion, the resin was cleaved from the solid support in a solution of trifluoroacetic acid (TFA), triisopropylsilane (TIPS), and water (TFA/TIPS/H₂O, 95:2.5:2.5, 500 μL/10 μmoles) for 2 h at 25° C. The resin was filtered off and the remaining solution was removed under nitrogen and a subsequent peptide precipitation step was conducted using cold ether. Centrifugation at 4° C. for 10 min at 13.3K rpm afforded the crude propargyl sulfonated peptide as a pellet. The RP-HPLC gradient used for preparative purposes included a solvent (A) comprised of water and 0.1% TFA and a solvent (B) consisting of acetonitrile: 0-2 min: 5% B, 2-5 min: 5-30% B, 5-16 min: 30-50% B, 16-17 min: 50-98% B, 17-18.5 min: 98% B, 18.5-20 min: 98-5% B. The propargyl sulfonated peptide displayed a retention time (Rt) of 9.60 min at a flow rate of 40 mL/min. The RP-HPLC gradient used for analytical purposes included a solvent (A) comprised of water and 0.1% TFA and a solvent (B) consisting of acetonitrile: 0-2 min: 5% B, 2-18 min: 5-95% B, 18-20 min: 95% B, 20-20.01 min: 95-5% B, 20.01-22 min: 5% B. The propargyl sulfonated peptide displayed a retention time (Rt) of 6.92 min at a flow rate of 2.5 mL/min. Preparative RP-HPLC afforded the propargyl sulfonated peptide in 58% yield.

### Synthesis of G4

Propargyl sulfonated peptide (1.4 equiv.) was dissolved in DMF with 1 M copper sulfate, 1 M tris(3-hydroxypropyltriazolylmethyl)amine (THPTA), and 1 M sodium ascorbate under argon. Immediately, a 1 M solution of P-2-azidoethyl sialyl Lewis^{X} (1 equiv.) in DMF in a separate Eppendorf tube was introduced to the reaction under argon. The reaction vessel was shaken and protected from light for 16 h at 25° C. Purity was assessed using RP-HPLC (analytical). The RP-HPLC gradient used for preparative purposes included a solvent (A) comprised of water and 0.1% TFA and a solvent (B) consisting of acetonitrile: 0-2 min: 5% B, 2-16 min: 5-98% B, 16-18 min: 98% B, 18-18.1 min: 98-5% B, 18.1-20 min: 5% B. G4 displayed a retention time (Rt) of 9.18 min at a flow rate of 40 mL/min. The RP-HPLC gradient used for analytical purposes included a solvent (A) comprised of water and 0.1% TFA and a solvent (B) consisting of acetonitrile: 0-2 min: 5% B, 2-18 min: 5-95% B, 18-20 min: 95% B, 20-20.01 min: 95-5% B, 20.01-22 min: 5% B. G4 displayed a retention time (Rt) of 6.17 min at a flow rate of 2.5 mL/min. Preparative RP-HPLC afforded the desired compound in 76% yield.

### Synthesis of PEG-G4 (P-G4)

G4 was dissolved in DMF (1.25 mM) and mPEG-SVA (1 equiv.) added. Subsequently, DIPEA was slowly added and shaken for 22 h at 25° C. The reaction was quenched using acetic acid and the crude material was purified using preparative RP-HPLC to afford the final product, P-G4 (P-G4), in 64% yield, which was confirmed by MALDI-TOF MS. Purity was assessed using analytical RP-HPLC. The RP-HPLC gradient used for preparative purposes included a solvent (A) comprised of water and 0.1% TFA and a solvent (B) consisting of acetonitrile: 0-10 min: 25% B, 10-40 min: 25-80% B, 40-41 min: 80-98% B, 41-50 min: 98% B, 50-51 min: 98-25% B, 51-60 min: 25% B. P-G4 displayed a retention time (Rt) of 20.13 min at a flow rate of 75 mL/min. The RP-HPLC gradient used for analytical purposes included a solvent (A) comprised of water and 0.1% TFA and a solvent (B) consisting of acetonitrile: 0-2 min: 5% B, 2-6 min: 5-98% B, 6-20 min: 98% B, 20-21 min: 98-5% B, 21-22 min: 5% B. P-G4 displayed a retention time (Rt) of 4.30 min at a flow rate of 2.5 mL/min.

### Microarray Printing, Binding Assay, and Scanning

For a multi-panel experiment on a single slide, the array layout was designed using Piezoarray software according to the dimension of a standard 14-chamber adaptor. The adaptor was applied on the slide to separate a single slide to 14 chambers sealed from each other during the experiment. All glycopeptides and control samples were printed on an NHS-activated glass slide in phosphate buffer (300 mM sodium phosphates, pH 8.5). The average spot volume was within 10% variation (intra-tip) of 0.33 nL. The average spot size was 100 μm. The following glycans were derivatized with 2-amino-N-(2-aminoethyl)-benzamide (AEAB): asialobiantennary N-glycan NA2 (Galβ1-4GlcNAcβ1-2Manα1-6(Galβ1-4GlcNAcβ1-2Manα1-3)Manβ1-4GlcNAcβ1-4GlcNAc), the 2,3-disialylated biantennary N-glycan NA2,3 (Neu5Acα2-3Galβ1-4GlcNAcβ1-2Manα1-6(Neu5Acα2-3Galβ1-4GlcNAcβ1-2Manα1-3)Manβ1-4GlcNAcβ1-4GlcNAc), the 2,6-disialylated biantennary N-glycan NA2,6 (Neu5Acα2-6Galβ1-4GlcNAcβ1-2Manα1-6(Neu5Acα2-6Galβ1-4GlcNAcβ1-2Manα1-3)Manβ1-4GlcNAcβ1-4GlcNAc), a sLe^{x} pentose sLe^{x}, and an lacto-N-neo-tetraose LNnT (Galβ1-4GlcNAcβ1-3Galβ1-4Glc) along with the following glycosulfopeptides bearing tyrosine sulfates (Y*) or phenylalanine sulfonates (F*): 4GSP-1 (EY*EY*LDY*DFLPET(GalNAc)EPPEM), 4GSP-6 (EY*EY*LDY*DFLPET(Neu5Acα2-3Galβ1-4(Fucal-3)GlcNAcβ1-6Galβ1-3GalNAcα)EPPEM), GSnP-3 (KEF*EF*LDF*DFLPET-(GlcNAcβ1-6(Galo 1-3)GalNAcα)EPL), GSnP-4 (KEF*EF*LDF*DFLPET(Galβ1-4GlcNAcβ1-6(Galβ1-3)GalNAcα)EPL), GSnP-5 (KEF*EF*LDF*DFLPET(Neu5Acα2-3Galβ1-4GlcNAcβ1-6(Galo 1-3)GalNAcα)EPL), GSnP-5b (KEF*EF*LDF*DFLPET(Galβ1-4(Fucal-3)GlcNAcβ1-6(Galβ1-3)GalNAcα)EPL), GSnP-6 (KEF*EF*LDF*DFLPET(Neu5Acα2-3Galβ1-4(Fucal-3)GlcNAcβ1-6(Galβ1-3)GalNAcα)EPL), GSnP-7 (KEF*EF*LDF*DFLPET(Neu5Acα2-3Galβ1-4(Fucal-3)GlcNAcβ1-6(Galβ1-3)GalNAcα)EPL), G1 (KEF*EF*LDF*DFLPET(GlcNAc)EPL), G2 (KEF*EF*LDF*DFLPET(Galβ1-4GlcNAc)EPL), G3 (KEF*EF*LDF*DFLPET(Neu5Acα2-3Galβ1-4GlcNAc)EPL), and G4 (KEF*EF*LDF*DFLPET(Neu5Acα2-3Galβ1-4(Fucal-3)GlcNAc)EPL). These AEAB derivatives and control biotin-NHNH₂, all at 50 μM concentration, were printed on the microarray as controls and reference standards. After printing, the slide was placed in a high moisture chamber at 50° C. and incubated for 1 h. The slide was subsequently washed and blocked with 50 mM ethanolamine in 0.1 M Tris buffer (pH 8.0) for 1 h, dried by centrifugation, and stored desiccated at −20° C. prior to use. The binding assay was performed by incubating the slide with human P-selectin (5 μg/mL in calcium buffer (pH 8.0), PL1, a monoclonal antibody specific to the N-terminal PSGL-1 peptide sequence, CHO131, a monoclonal antibody specific to the Core 2 O-glycan terminated sLe^{x}, or HECA-452, a monoclonal antibody specific to sLe^{x}. Binding is Ca²⁺ dependent and was quantitatively inhibited by addition of EDTA. Detection was accomplished using Alexa 488 goat anti-human IgG (5 μg/mL). The slide was subsequently washed (TSM Buffer, five times), centrifuged and analyzed with a Perkin Elmer ProScanArray microarray scanner equipped with four lasers covering an excitation range from 488 to 637 nm. The scanned images were analyzed using ScanArray Express software.

### Surface Plasma Resonance Binding Assay of G4

Biotinylated G4 was first captured quantitatively on a streptavidin-coated sensor chip (GE Healthcare). Different amounts of P-selectin-Ig chimera (P-Sel-Ig) were incubated in the wells (0.4-100 nM) and bound P-selectin detected using fluorescently labelled anti-human IgG. PL1 and CHO131 were used to confirm the immobilization step and to compare the relative amount of glycopeptide on the chip. Bound monoclonal antibodies and P-selectin were removed from the sensor chip by injecting 10 mM glycine-HCl, pH 2.0 and 50 mM EDTA, respectively, at 20 μL/min for 30 s. The kinetic parameters were obtained by plotting the curves using a 1:1 binding model provided by the Biacore evaluation software. P-Sel-Ig generated a saturated binding curve irrespective of the G4-coated densities used (<5-60 RU), indicating that affinity of P-Sel-Ig for G4 is not dependent on the density of the immobilized ligand.

### Flow Cytometry

Flow cytometry was used to quantify binding inhibition of P-selectin-Fc chimeras to human and murine leukocytes. Heparinized whole mouse blood was collected via cardiac puncture and human blood from healthy adult volunteers was collected in citrate. Leukocytes were isolated by centrifugation at 800 g for 10 min at room temperature, platelets removed by centrifugation at 120 g for 10 min at room temperature, and red blood cells lysed using red blood cell (RBC) lysis buffer (eBiosciences). A total of 3×10⁵ leukocytes were incubated with increasing concentrations of G4 or P-G4 (0-100 μM) and human or murine P-selectin-Fc chimera (3 μg/mL; R&D Systems) followed by phycoerythrin (PE)-conjugated anti-Fc antibody (1:100; Life Technologies) at 4° C. The interaction of P-selectin with human and murine monocytes and neutrophils was analyzed by flow cytometry (CytoFlex LX, Beckman Coulter) and quantified as percent inhibition using FlowJo. Inhibition experiments were conducted in triplicate.

### Assessment of Platelet-Leukocyte Aggregation In Vitro

Platelet-leukocyte aggregates were quantified in whole blood using dual-label flow cytometry (CytoFlex LX, Beckman Coulter). Whole human and mouse blood was collected into citrate coated tubes, incubated with 120 μM of G4 or P-G4 at room temperature, and stimulated with thrombin receptor-activating peptide (human PAR₁-activating peptide 40 μM; mouse PAR4-activating peptide 200 μM) to induce platelet P-selectin expression. Platelet-leukocyte aggregates were quantified by two-color flow cytometry by incubating human samples with anti-CD45-APC (BD Biosciences) and anti-CD42a-FITC (BD Biosciences) and mouse samples with anti-CD45-eFluor506 (Invitrogen), anti-CD11b-APC (BD Biosciences), anti-Ly-6G-PE (BD Biosciences), and anti-CD41-FITC (BD Biosciences). CD45+ monocytes and neutrophils were discerned through characteristic side scatter and quantified as % platelet positive in saline, G4 (120 μM), or P-G4 (120 μM). Inhibition experiments were conducted in triplicate.

### Pharmacokinetic Profile

G4 or P-G4 was administered intravenously to C57BL/6 mice at a dose of 8 μmol/kg and blood collected in lithium heparin tubes and centrifuged at 1,500 g for 10 min at room temperature. G4 or P-G4 was extracted from 20 μL of plasma by protein precipitation using 80 μL cold methanol (Fisher Scientific) and centrifuged at 17,000 g for 20 min at 4° C. The supernatant (80 μL) was dried in vacuo, reconstituted in nanopure water (32 μL), and an aliquot (2 μL) was injected for pre-concentration at a flow rate of 5 μL/min using an UltiMate 3000 RSLCnano ultra-high-performance liquid chromatography (UHPLC) system (Dionex) coupled to an Orbitrap Fusion Lumos mass spectrometer (Thermo Fisher Scientific). A binary gradient was applied to an Accucore™ 150 amide hydrophilic interaction liquid chromatography (HILIC) analytical column (150 mm×75 μm ID, 2.6 m; Thermo Fisher Scientific) maintained at 40° C. using (A) 5 mM ammonium formate (Honeywell Fluka) in water (pH 8.5) and (B) 5 mM ammonium formate (Honeywell Fluka) in 80% (v/v) acetonitrile and 20% (v/v) water (pH 8.5) at a flow rate of 0.4 μL/min: 0-3 min: 99% (B), 3-14 min: 99-89% (B), 14-26 min: 89-45% (B), 26-33 min: 45% (B), 33-34 min: 45-99% (B), 34-44 min: 99% (B). For P-G4 analysis, an offset voltage (30 V) was applied in the ion source post-LC separation. Mass spectrometry (MS) and tandem mass spectrometry (MS/MS) spectra were acquired in the negative mode with the following settings: RF lens, 24%; MS automatic gain control, 5×10⁵; MS maximum injection time, 50 ms; MS resolution, 120,000; MS/MS automatic gain control, 2×10⁵; MS/MS maximum injection time, 200 ms; MS/MS resolution, 30,000; precursor ion isolation width, 1.6 Da; and higher-energy collisional dissociation normalized collision energy, 20, 30, 40. Following in-source fragmentation (ISF), m/z 1121.09 (z=3) was used as the quantifying peptide. Peak areas from the extracted chromatograms were used for quantification. An external standard curve was generated by spiking plasma with varying concentrations of G4 or P-G4 followed by protein precipitation, as described. A linear regression curve was fitted to the data and used to determine concentrations of G4 or P-G4 in collected plasma samples. Compound half-life was determined using non-compartmental analysis.

### Murine Model of Non-Occlusive Venous Thrombosis

Drug efficacy was evaluated in a preclinical mouse model in which non-occlusive venous thrombosis was induced by electrolytic injury of the inferior vena cava, as detailed elsewhere (58). P-G4 was administered intravenously (8 μmol/kg) to male C57BL/6 mice (8-12 weeks of age) immediately prior to electrolytic injury and two hours after injury. Enoxaparin was administered subcutaneously (6 mg/kg) 4 hours prior and 24 hours after electrolytic injury as a clinically relevant control. C57BL/6 mice (Jackson Labs, Bar Harbor, ME, USA) were anesthetized with 2% isoflurane and the inferior vena cava approached via a midline laparotomy. Venous side branches were ligated or cauterized, while posterior branches were left patent. A 25-gauge stainless steel needle, attached to a silver-coated copper wire was inserted into the exposed caudal vena cava and positioned against the anterior wall (anode). A second wire was implanted subcutaneously to complete the circuit (cathode) and a 250 μAmps current applied for 15 min. Subsequently, the needle was removed and a cotton swab held in gentle contact with the puncture site to prevent bleeding. The vena cava and associated thrombus, immediately below the renal veins to just above the bifurcation, was excised 48 hours after injury for determination of wet thrombus weight.

Tail Vein Transection Bleeding Time Hemostasis was assessed using a tail vein transection model to determine bleeding time (58). Mice were anesthetized with ketamine and xylazine by intraperitoneal injection and placed on a warming mat at 37° C. Sterile saline, enoxaparin (6 mg/kg), or P-G4 in 125 μL of sterile saline was injected into the penile vein. Five minutes after administration of test compound, the lateral tail vein was transected with a number 11 scalpel blade at a tail width of 2.3 mm and immediately submerged in 37° C. phosphate buffered saline. The bleeding time was determined at that instant when bleeding had ceased for 30 seconds. Animals were excluded if arterial bleeding was present.

### Statistical Analysis

Descriptive data are presented as mean±SEM unless otherwise stated. Group comparisons were conducted using one-way ANVOA with Tukey's multiple comparison or Welch's ANOVA with Dunnet's multiple comparison as appropriate for variance of data. Statistical analysis was performed using GraphPad Prism version 8.0.0 for Windows (GraphPad Software, San Diego, CA).

### Methyl (phenyl 5-acetamido-7,8,9-tri-O-acetyl-5-N,4-O-carbonyl-3,5-dideoxy-2-thio-D-glycero-β-D-galacto-non-2-ulopyranoside)onate (9a)

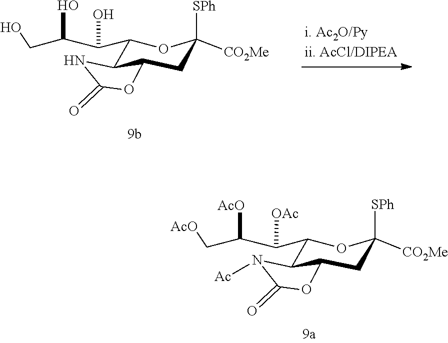

To a solution of 9b (1.0 g, 2.50 mmol) in pyridine (3.0 mL) was added Ac₂O (1.5 mL) at room temperature. The resulting reaction mixture was stirred for 15 h and the solvent was removed in vacuo until half of the volume was reached. Then the crude mixture was diluted in EtOAc, washed with 1N HCl, sat. NaHCO₃, brine solution and concentrated to give partial acetylated sialic acid derivative in a thick gum form, which was taken to the next step without further purification. The solution of the resulting thick mass in anhyd. CH₂Cl₂ (10.0 mL) was treated with N,N-diisopropylethylamine (3.4 g, 0.026 mmol, 10.0 equiv.) and acetyl chloride (1.64 g, 0.021 mmol, 8 equiv.) at ice cooled temperature. The resulting reaction mixture was stirred for 2 h before being poured into a sat. NaHCO₃ solution. The compound was extracted into CH₂Cl₂ and washed with water, brine solution, and concentrated under reduced pressure. The residue was purified by column chromatography using a EtOAc:hexanes (2:3) elution system to afford the desired sialic acid derivative 9a (1.25 g, yield: 88%) as a white foam.

The spectral data was consistent with previously reported data.⁷⁴

### Methyl (5-acetamido-7,8,9-tri-O-acetyl-5-N,4-O-carbonyl-2-(dibutylphosphoryl)-3,5-dideoxy-D-glycero-α,β-D-galacto-non-2-ulopyranoside)onate (9)

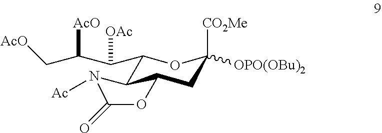

A solution of sialic acid-oxazolidinone derivative 9a (600.0 mg, 1.08 mmol, 1.0 equiv.), dibutyl phosphate (1.12 g, 5.33 mmol, 5.0 equiv.), and activated 4 Å powdered molecular sieves (600.0 mg) in anhydrous CH₂Cl₂ (1.4 mL) was stirred for 1 h under Argon, and then cooled to 0° C. followed by addition of NIS (467.0 mg, 2.075 mmol, 2.0 equiv.) and TfOH (63.5 mg, 0.423 mmol, 0.4 equiv.). After 20 min, a second lot of TfOH (63.5 mg, 0.423 mmol, 0.4 equiv.) was added and the resulting reaction mixture was stirred for 3 h at 0′C and then quenched with sat. Na₂S₂O₃ and sat. NaHCO₃ solutions. (TLC system: 60% EtOAc:Hexanes). The mixture was diluted with CH₂Cl₂, washed with sat. NaHCO₃ solution and brine, filtered through a pad of celite, and concentrated under reduced pressure. The residue was purified by column chromatography on silica gel eluting with a EtOAc:hexanes system to afford the desired sialyl-phosphate donor 9 (520.0 mg, yield: 73%) as a white foam in 1:1 ratio. ¹H NMR (400 MHz, Chloroform-d) δ 5.67-5.55 (m, 2H), 5.32-5.17 (m, 2H), 4.70 (ddd, J=11.8, 9.5, 1.8 Hz, 2H), 4.60-4.45 (m, 2H), 4.37 (dd, J=12.3, 2.8 Hz, 1H), 4.20-4.08 (m, 2H), 4.04 (dtd, J=9.6, 5.6, 4.6, 2.8 Hz, 5H), 3.86-3.77 (m, 5H), 3.74 (dd, J=11.3, 9.5 Hz, 1H), 2.96 (dd, J=12.2, 4.0 Hz, 1H), 2.85 (dd, J=12.7, 3.7 Hz, 1H), 2.70-2.59 (m, 1H), 2.26 (td, J=12.8, 2.9 Hz, 1H), 2.17-2.03 (m, 8H), 1.99 (d, J=3.5 Hz, 4H), 1.64 (ddt, J=14.7, 8.9, 5.3 Hz, 6H), 1.37 (hept, J=7.3 Hz, 6H), 1.21 (d, J=2.7 Hz, 2H), 0.90 (td, J=7.4, 1.9 Hz, 9H). ¹³C NMR (100 MHz, Chloroform-d) δ 172.05, 171.73, 170.50, 170.47, 169.89, 169.77, 169.66, 167.24, 167.17, 165.47, 153.45, 153.41, 98.82, 98.76, 98.17, 98.11, 77.37, 77.16, 77.05, 76.73, 76.57, 74.13, 73.96, 72.49, 71.71, 71.43, 69.76, 68.48, 68.42, 68.32, 68.27, 68.08, 68.02, 67.97, 67.90, 62.78, 62.46, 58.84, 58.28, 53.42, 53.40, 53.37, 36.04, 35.98, 35.86, 35.82, 32.12, 32.09, 32.05, 32.02, 31.99, 29.62, 24.56, 24.54, 20.93, 20.89, 20.72, 20.66, 18.58, 18.55, 18.53, 13.50, 13.47; [α]_{D}²⁶=+15.0 (c=0.40, CH₂Cl₂); R_{f}=0.40 (EtOAc, Hexanes; 3:2); MALDI-TOF/MS (positive, SuperDHB matrix): m/z calcd for C₂₇H₄₂NNaO₁₆P [M+Na]⁺ 690.2139, observed: 690.2149.

### Phenyl 4,6-O-benzylidene-2-O-benzoyl-1-thio-β-D-galactopyranoside (10)

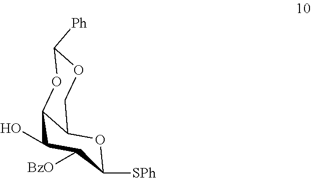

The galactose derivative was synthesized by using a reported protocol and the spectral data was consistent with previously reported data.^{73 1}H NMR (400 MHz, Chloroform-d) δ 8.10-8.08 (m, 2H), 7.60-7.58 (m, 3H), 7.49-7.39 (m, 7H), 7.34-7.26 (m, 3H), 5.54 (s, 1H), 5.28 (t, J=10.0 Hz, 1H), 4.83 (d, J=10.0 Hz, 1H), 4.42 (dd, J=8.4, 1.2 Hz, 1H), 4.26 (d, J=3.2 Hz, 1H), 4.05 (dd, J=12.4, 1.6 Hz, 1H), 3.91 (m, 1H), 3.60 (s, 1H), 2.71 (d, J=10.8 Hz, 1H); ¹³C (100 MHz, Chloroform-d) δ 166.0, 137.4, 133.9, 133.2, 131.1, 129.92, 129.86, 129.4, 128.8, 128.4, 128.2, 126.6, 101.45 (7-C), 84.9 (—C), 75.7 (4-C), 72.9 (3-C), 70.7 (2-C), 70.0 (5-C), 69.1 (6-C); [α]_{D}²⁶=+32.0 (c=0.20, CH₂Cl₂); ESI/Mass: m/z calcd for C₂₆H₂₄NaO₆S [M+Na]⁺ 487.1191, observed: 487.1188.

### 2-Azidoethyl 6-tert-O-butyldiphenylsilyl-2-deoxy-2-phthalimido-β-D-glucopyranoside (13)

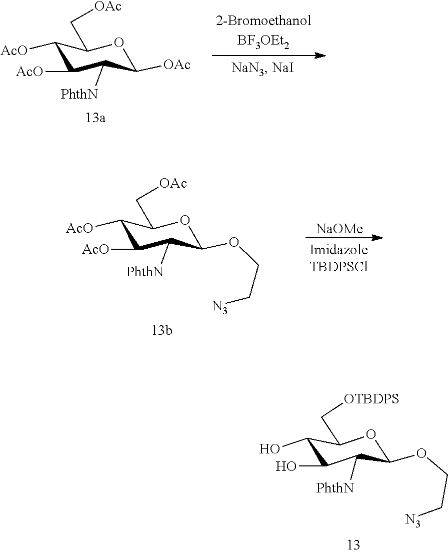

An oven-dried 100 mL round-bottomed flask was charged with tetraacetate 13a⁷¹ (2.0 g, 4.19 mmol, 1.0 equiv.), 2-bromoethanol (2.6 g, 20.8 mmol, 5.0 equiv.), and anhyd CH₂Cl₂ (10 mL). The mixture was treated with boron trifluoride etherate (0.9 g, 6.29 mmol, 1.5 equiv.) at ice-cooled temperature and stirred for 15 h under argon. The mixture was then diluted with CH₂Cl₂ and washed with water, brine and concentrated by rotary evaporation. The residue was dried in high vacuum for 2 h before start the next reaction. The resultant crude was dissolved in anhydrous DMF, treated with sodium azide (1.36 g, 21.00 mmol, 5.0 equiv.) and sodium iodide (0.629 g, 4.19 mmol, 1.0 equiv.) at 80° C. and stirred for 3 h. The reaction mixture was diluted with CH₂Cl₂ and washed with water twice, brine solution and solvent evaporated under reduced pressure. The residue was purified by column chromatography on silica gel eluting with EtOAc:hexanes systems to afford the desired 2-azidoethyl glucosamine derivative 13b (1.35 g, yield: 64%) as a white foam. The spectral data was consistent with previously reported data.

¹H NMR (400 MHz, Chloroform-d) δ 7.84 (dd, J=5.5, 3.0 Hz, 2H, Arom.), 7.75-7.66 (m, 5H, Arom.), 7.51-7.36 (m, 5H, Arom.), 5.29 (d, J=8.4 Hz, 1H, H-1), 4.36 (ddt, J=10.7, 8.4, 3.7 Hz, 1H, H-3), 4.16 (dd, J=10.9, 8.4 Hz, 1H, 6-CH₂), 4.03-3.86 (m, 3H, 6-CH₂), 3.76-3.66 (m, 1H, H-4), 3.65-3.52 (m, 2H, CH₂CH₂N₃), 3.33 (ddd, J=13.4, 8.1, 3.6 Hz, 1H, CH₂CH₂N₃), 3.22 (d, J=2.3 Hz, 1H, H-5), 3.13 (ddd, J=13.3, 5.2, 3.5 Hz, 1H, CH₂CH2N₃), 2.52 (s, 1H, OH), 1.08 (s, 9H, TBDPS). ¹³C NMR (100 MHz, Chloroform-d) δ 153.96, 135.57, 134.01, 132.71, 132.56, 131.78, 129.98, 127.86, 98.05 (1-C), 74.34 (C-4, CH₂), 71.77 (C-3), 68.14, 50.41, 26.79 (C-t-Butyl ‘C’), 19.19 (C—CH₃); [α]_{D}²⁸=−29.33 (c=0.02, CH₂Cl₂); HR-MALDI-TOF/MS (positive, SuperDHB matrix): m/z calcd for C₃₂H₃₆N₄NaO₇S [M+Na]⁺ 639.2251, observed: 639.2248.

### Ethyl 2-p-methoxybenzyl 3,4-di-O-acetyl-1-thio-α-L-fucopyranoside (15)

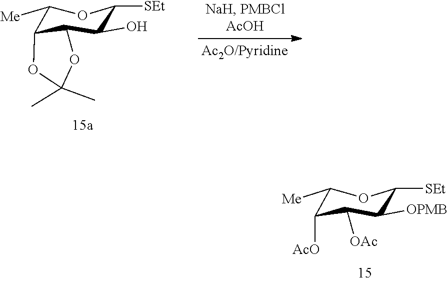

To a 100 mL round-bottomed flask was added the isopropylidine fucose derivative 15a^{72,70} (1.00 g, 2.71 mmol, 1.0 equiv.) and anhydrous DMF (20.0 mL). The mixture was treated with NaH (500.0 mg, 60%) and stirred for 10 min at room temperature. Then PMBCl (1.625 mL, 11.98 mmol, 4.5 equiv.) was added in drop wise manner at ice cooled temperature and the resulting reaction mixture was stirred for 2 h before quenching with methanol. The reaction mixture was diluted with CH₂Cl₂ and washed with water and brine. The crude product was concentrated under reduced pressure and purified by silica gel chromatography using an EtOAc-hexanes gradient to yield the desired PMB protected fucose derivative as a white foam (1.15 g, yield: 78%). Without further characterization, this compound was subjected to next reaction. A solution of PMB protected fucose derivative (1.15 g, 3.125 mmol, 1.0 equiv.) in 80% AcOH was heated to 45 t for 2 h before solvent was evaporated under reduced pressure. The crude product was subjected to next reaction after drying for 6 h. A solution of the crude product (1.14 g) in pyridine (8.0 mL) was treated with acetic anhydride (1.2 mL) and DMAP (40 mg). The reaction mixture was stirred for 1 h before quenching with ethanol. The solvents were evaporated under reduced pressure and purified by silica gel chromatography using a EtOAc-hexanes gradient to yield the desired fucose donor 15 as a white foam (1.33 g, 95.0% yield). ¹H NMR (400 MHz, Chloroform-d) δ 7.21 (d, J=8.3 Hz, 2H), 6.80 (d, J=8.4 Hz, 2H), 5.21-5.07 (m, 1H, H-3), 5.01-4.84 (m, 1H, H-4), 4.71 (d, J=10.5 Hz, 1H, PMBCH₂), 4.50 (d, J=10.5 Hz, 1H, PMBCH₂), 4.45 (d, J=10.5 Hz, 1H, H-1) 3.72 (S, 3H, OCH₃), 3.67-3.51 (m, 2H, H-5, H-2), 2.85-2.60 (m, 2H, SCH₂CH₃), 2.09 (s, 3H, CH₃), 1.92 (s, 3H, CH₃), 1.27 (t, J=7.5 Hz, 3H, SCH₂CH₃), 1.11 (d, J=6.4 Hz, 3H, 6-CH₃). ¹³C NMR (100 MHz, Chloroform-d) δ 170.50, 169.87, 159.30, 130.06, 129.63, 113.67, 113.65, 84.97 (C-1), 75.73 (C-2), 75.02 (C—CH₂PMB), 74.48 (C-4), 72.62 (C-5), 70.97 (C-3), 55.30 (C—OCH₃), 25.00 (SCH₂CH₃), 20.70 (OAc), 20.63 (OAc), 14.91 (C-6), 14.88 (SCH₂CH₃). [α]_{D}²⁶=+82.5 (c=0.20, CH2Cl2); ESI/MS: m/z calcd for C₂₀H₂₈NaO₇S [M+Na]⁺ 435.145, found: 435.166.

### Methyl (5-acetamido-7,8,9-tri-O-acetyl-5-N,4-O-carbonyl)-3,5-dideoxy-D-glycero-α,β-D-galacto-non-2-ulopyranoside)onate-(2→3)-phenyl-2-O-benzoyl-4,6-O-benzylidene-1-thio-β-D-galactopyranoside (11)

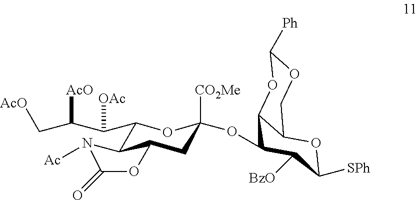

A solution of sialic acid-phosphate donor 9 (520.0 mg, 0.779 mmol, 1.0 equiv.), galactose acceptor 10 (230 mg, 0.495 mmol, 0.66 equiv.), and activated 4 Å powdered molecular sieves (˜500.0 mg) in anhydrous CH₂Cl₂ (7.0 mL) was stirred for 1 h under an Ar, and then cooled to −78° C. followed by addition of TMSOTf (173.0 mg, 0.779 mmol, 1.0 equiv.) under argon atmosphere. The resulting reaction mixture was stirred for 1 h at −78° C. and another 1 h at −60° C. before quenching with TEA (1.5 equiv.). (TLC system: 20% acetone:toluene). The mixture was diluted with CH₂Cl₂, then filtered through celite, and concentrated under reduced pressure. The residue was purified by column chromatography on silica gel eluting with EtOAc:hexanes systems to afford the desired sialyl-galactose disaccharide 11 (380.0 mg, yield: 75%) as a white foam. ¹H NMR (400 MHz, Chloroform-d) δ 8.18-8.08 (m, 2H), 7.63-7.53 (m, 3H), 7.52-7.43 (m, 2H), 7.43-7.31 (m, 3H), 7.31-7.16 (m, 3H), 5.54 (m, 2H, 8′-H, 7′-H), 5.42 (t, J=9.8 Hz, 1H, 2-H), 5.35 (s, 1H, Benzylidene-H), 4.97 (d, J=9.8 Hz, 1H, 1-H), 4.60 (dd, J=9.7, 3.5 Hz, 1H, 3-H), 4.48-4.45 (m, 1H, 9′-CH₂), 4.43 (br S, 1H, 6′-H), 4.36 (dd, J=12.2, 1.6 Hz, 1H, 6-CH₂), 4.16-4.11 (m, 1H, 6-CH₂), 4.11-4.09 (m, 1H, 4-H), 3.99 (m, 1H, 9′-CH₂), 3.76-3.71 (m, 1H, 4′-H), 3.69 (m, 1H, 5-H), 3.54-3.46 (dd, J=12.0, 4.0 Hz, 1H, 5′-H), 3.45 (s, 3H, CO₂CH₃), 2.89 (dd, J=12.0, 3.2 Hz, 1H, 3′-CH₂), 2.43 (s, 3H, CH₃), 2.19 (s, 3H, CH₃), 2.02 (s, 3H, CH₃), 1.80 (s, 3H, CH₃), 1.73 (t, J=12.0 Hz, 1H, 3′-CH₂). ¹³C NMR (100 MHz, Chloroform-d) δ 171.88, 170.85, 170.39, 170.02, 168.68, 164.85, 153.36 (7C, C═O), 137.76, 133.85, 133.09, 131.33, 130.39, 129.89, 129.03, 128.63, 128.43, 128.09, 127.94, 126.56 (Arom-C), 100.87 (Benzylidene-C), 96.77 (2′-C), 85.04 (1-C), 74.94 (6′-C), 74.89 (4′-C), 73.77 (3-C), 72.91 (4-C), 71.43 (7′-C), 69.50 (5-C), 69.16 (6-CH₂), 68.21 (2-C), 67.99 (8′-C), 63.74 (9′-CH₂), 58.81 (5′-C), 52.81 (CO₂CH₃), 37.00 (3′-C), 24.58, 21.36, 20.81, 20.52 (4C's-CH₃CO). [α]_{D}²⁶=+33.0 (c=0.20, CH₂Cl₂); R_{f}: 0.40 (20% Acetone, Toluene); HR-MALDI-TOF/MS (positive, SuperDHB matrix): m/z calcd for C₄₅H₄₇NNaO₁₈S [M+Na]⁺ 944.2412, found: 944.2406.

### Methyl (5-acetamido-7,8,9-tri-O-acetyl-5-N,4-O-carbonyl)-3,5-dideoxy-D-glycero-α,β-D-galacto-non-2-ulopyranoside)onate-(2→3)-phenyl-2-O-benzoyl-4,6-O-benzylidene-1-thio-β-D-galactopyranosyl dibutyl phosphate (12)

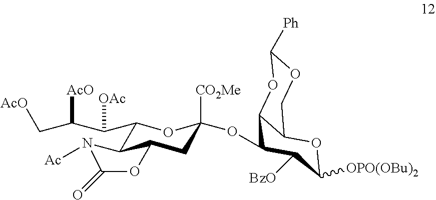

A solution of Sia-Gal derivative 11 (250.0 mg, 0.271 mmol, 1.0 equiv.), dibutyl phosphate (171.0 mg, 0.814 mmol, 3.0 equiv.), and activated 4 Å powdered molecular sieves (300.0 mg) in anhydrous CH₂Cl₂ (10.0 mL) was stirred for 1 h under an Ar, and then cooled to 0° C. followed by addition of NIS (122.0 mg, 0.542 mmol, 2.0 equiv.) and TfOH (8.1 mg, 0.054 mmol, 0.2 equiv.). After 20 min, second lot of TfOH (8.1 mg, 0.054 mmol, 0.2 equiv.) was added and the resulting reaction mixture was stirred for 10 min at 0° C. before pouring into sat. NaHCO₃ solution (TLC system: 60% EtOAc:Hexanes). The mixture was diluted with CH₂Cl₂, washed with sat. Na₂S₂O₃ solution and brine, then filtered through a celite bed, and concentrated under reduced pressure. The residue was purified by column chromatography on silica gel eluting with EtOAc:hexanes systems to afford the desired Sialic-Gal-phosphates 12 (200.0 mg, Yield: 72%) as a white foam in 1:1 ratio. ¹H NMR (400 MHz, Chloroform-d) δ 8.15-8.09 (m, 2H), 7.60-7.50 (m, 3H), 7.48-7.41 (m, 2H), 7.41-7.34 (m, 3H), 5.67-5.53 (m, 2H, H-1, H-2), 5.48 (m, 1H, H-7′), 5.40 (s, 1H, Benzylidine), 4.60 (dd, J=9.7, 3.5 Hz, 1H, H-3), 4.48 (m, 1H, H-4′), 4.42 (m, 1H, 9′-CH₂), 4.32-4.15 (m, 3H, H-4, 6-CH₂), 4.13-3.99 (m, 3H) 3.81-3.61 (m, 4H, H-4), 3.52 (t, J=8.0 Hz, 1H, H-5), 3.44 (s, 3H, CO₂Me), 2.92 (dd, J=12.1, 3.2 Hz, 1H, H-3′), 2.44 (s, 3H, CH₃), 2.18 (s, 3H, CH₃), 2.06 (s, 3H, CH₃), 1.94 (s, 3H, CH₃), 1.77 (t, J=12.1 Hz, 1H, H-3′), 1.68-1.56 (m, 2H, CH₂), 1.48-0.63 (several multiplets, t-Bu). ¹³C NMR (100 MHz, Chloroform-d) δ 171.80, 170.86, 170.26, 169.96, 168.50, 165.07, 153.37, 137.72, 133.32, 129.82, 129.03, 128.47, 128.15, 126.40, 100.69, 100.63 (Benzylidine C's), 96.90 (C-1), 96.79 (C-2′), 74.98 (C-4′), 72.51, 72.23 (C-2), 71.33 (C-2), 70.06, 69.97, 68.71, 68.04, 67.77, 67.70, 66.93, 63.48, 58.87 (C-5′), 52.93 (CO₂CH₃), 38.72, 37.02 (C-3′), 32.01, 31.94, 31.76 (C-3′CH₂), 31.68, 30.34, 28.90, 24.58, 23.73, 22.96, 21.36, 21.32, 20.83, 20.68, 18.54, 18.19, 14.02, 13.51, 13.33; ³¹P NMR (162 MHz, Chloroform-d) 6-2.78; [α]_{D}²⁶=+12.5 (c=0.15, CH2Cl2); R_{f}: 0.22 (EtOAc, Hexanes; 3:2); HR-MALDI-TOF/MS (positive, SuperDHB matrix): m/z calcd for C₄₇H₆₀NNaO₂₂P [M+Na]⁺ 1044.3242, found: 1044.3246.

### 2-Azidoethyl [Methyl (5-acetamido-7,8,9-tri-O-acetyl-5-N,4-O-carbonyl)-3,5-dideoxy-D-glycero-α,β-D-galacto-2-nonulopyranonate]-(2→3)-2-O-benzoyl-4,6-O-benzylidene-β-D-galactopyranosyl-(1→4)-6-O-tert-butyldiphenylsilyl-2-deoxy-2-phthalimido-β-D-glucopyranoside (14)

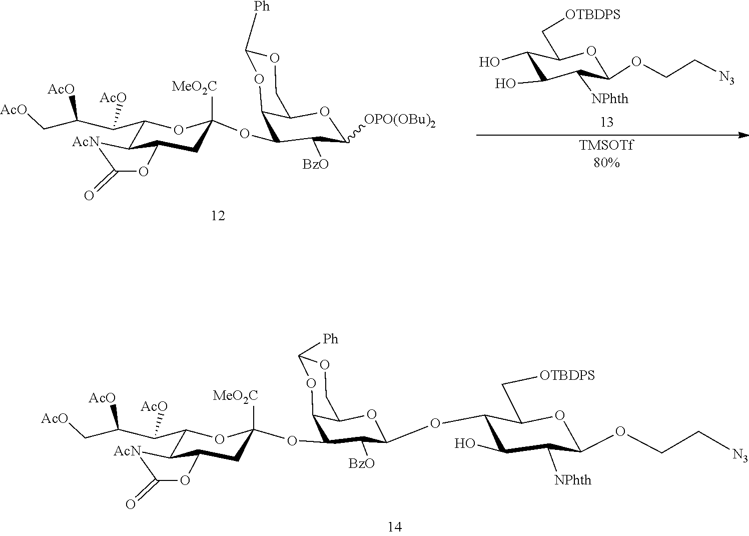

A solution of Sia-Gal phosphate donor 12 (187.0 mg, 0.183 mmol, 1.0 equiv.), Glc diol 13 (90.0 mg, 0.146 mmol, 0.8 equiv.), and activated 4 Å powdered molecular sieves (200 mg) in anhydrous CH₂Cl₂ (8.0 mL) was stirred for 1 h under Ar, and then cooled to −10° C. followed by addition of TMSOTf (33 μL, 0.183 mmol, 1.0 equiv.). The reaction mixture was stirred at −10′C to −5° C. for 30 min and then quenched with TEA (1.5 equiv.) (TLC system: 60% EtOAc:Hexane). The mixture was diluted with CH₂Cl₂, filtered through a pad of celite, and concentrated under reduced pressure. The residue was purified by column chromatography on silica gel eluting with EtOAc:hexanes systems to afford the desired trisaccharide 16 (209.0 mg, yield: 80%) as a white foam. ¹H NMR (600 MHz, Chloroform-d) δ 7.99-7.94 (m, 2H), 7.83 (q, J=5.7 Hz, 2H), 7.72-7.66 (m, 4H), 7.65-7.60 (m, 2H), 7.54 (ddd, J=7.4, 6.1, 1.3 Hz, 1H), 7.53-7.47 (m, 2H), 7.47-7.27 (m, 11H), 5.63-5.57 (m, 2H, H-2′, H-7″), 5.55 (ddd, J=9.8, 7.8, 2.4 Hz, 1H), 5.39 (s, 1H, Benzylidene), 5.25 (d, J=8.5 Hz, 1H, H-1), 4.99 (d, J=8.0 Hz, 1H, H-1′), 4.57-4.47 (m, 2H, H-5, H-3′), 4.48 (dd, J=10.9, 8.4 Hz, 1H, H—CH₂), 4.42 (dd, J=12.2, 2.5 Hz, 1H, H—CH₂), 4.34-4.13 (m, 4H, H-2, 6′-CH₂), 4.03 (dd, J=12.2, 7.8 Hz, 1H, CH₂), 3.93 (dd, J=9.7, 8.4 Hz, 1H, H-4), 3.83-3.64 (m, 4H, 6-CH₂), 3.60-3.47 (m, 2H, 6-CH₂), 3.42 (ddd, J=10.7, 7.9, 3.6 Hz, 1H, H-5), 3.33 (s, 3H, CO₂Me), 3.24 (ddd, J=13.3, 7.9, 3.7 Hz, 1H, CH₂), 3.05 (ddd, J=13.3, 5.3, 3.6 Hz, 1H, CH₂), 2.91 (dd, J=12.0, 3.3 Hz, 1H, H-3″), 2.48 (s, 3H, NAc), 2.20 (s, 3H, CH₃), 2.02 (d, J=3.3 Hz, 6H, CH₃), 1.67 (t, J=12.0 Hz, 1H, H-3″), 1.06 (s, 9H, TBDPS). ¹³C NMR (151 MHz, Chloroform-d) δ 168.35, 165.00, 135.81, 135.64, 133.82, 133.25, 133.05, 129.66, 129.61, 129.59, 129.01, 128.53, 128.11, 127.62, 126.50, 100.96 (C-1′), 100.53 (C-Benzylidine), 97.77 (C-1), 96.96 (C-2″), 80.43 (C-4), 77.27, 77.05, 76.84, 75.07, 74.95, 74.82, 72.68, 72.13, 71.48, 70.26 (C-3), 69.76, 68.38, 67.86 (6′-CH₂), 67.79 (CH₂CH₂N₃), 66.56, 63.84 (CH₂), 62.49 (CH₂), 58.93, 56.03 (C-2), 52.91, 50.32 (CH₂), 37.33 (CH₂), 26.91, 24.68, 21.34, 20.89, 20.83, 19.47. [α]_{D}²⁸=+8.0 (c=0.02, CH₂Cl₂); R_{f}=0.32 (EtOAc, Hexanes; 3:2); HR-MALDI-TOF/MS (positive, SuperDHB matrix): m/z calcd for C₇₁H₇₇N₅NaO₂₅Si [M+Na]⁺ 1450.4575, found: 1450.4568.

### 2-Azidoethyl [Methyl (5-acetamido-7,8,9-tri-O-acetyl-5-N,4-O-carbonyl)-3,5-dideoxy-D-glycero-α,β-D-galacto-2-nonulopyranonate]-(2→3)-2-O-benzoyl-4,6-O-benzylidene-β-D-galactopyranosyl-(1->4)[(1→3)-2-p-methoxybenzyl 3,4-di-O-acetyl-α-L-fucopyranosyl]-6-O-tert-butyldiphenylsilyl-2-deoxy-2-phthalimido-β-D-glucopyranoside (16)

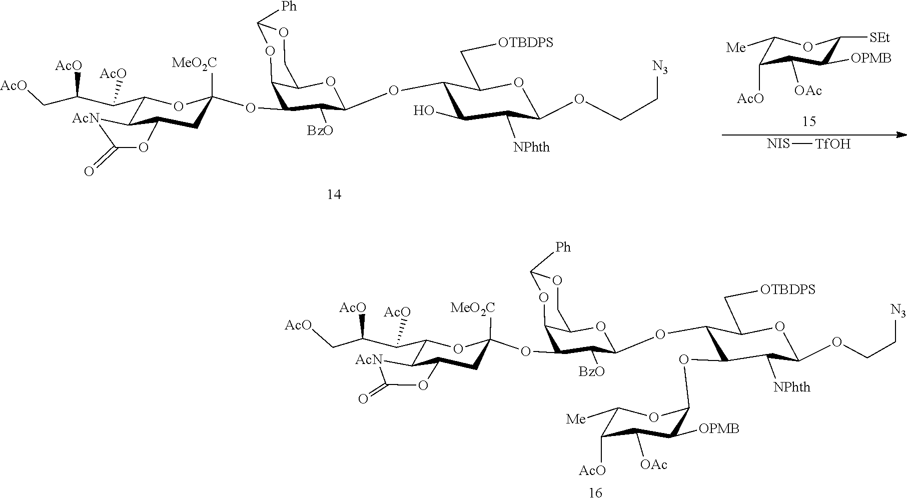

A solution of trisaccharide acceptor 14 (120.0 mg, 0.084 mmol, 1.0 equiv.), fucosyl donor 15 (173.0 mg, 0.420 mmol, 5.0 equiv.), and activated 3 Å powdered molecular sieves (˜120 mg) in anhydrous CH₂Cl₂ (1.4 mL) and Et₂O (2.8 mL) was stirred for 1 h under an Ar, and then cooled to −50′C followed by addition of NIS (95.0 mg, 0.422 mmol, 5.0 equiv.) and TfOH (13.0 μL, 0.147 mmol, 1.7 equiv.). The reaction mixture was stirred at −50° C. to −40° C. for 30 min and then quenched with excess TEA (TLC system: 20% Acetone:Toluene). The mixture was diluted with CH₂Cl₂, washed with sat. Na₂S₂O₃ and brine, then filtered through Celite, and concentrated under reduced pressure. The residue was purified by column chromatography on silica gel eluting with EtOAc:hexanes systems to afford the desired tetrasaccharide 16 (138.0 mg, yield: 85%) as a white foam. ¹H NMR (600 MHz, Chloroform-d) δ 7.89-7.76 (m, 8H), 7.72 (d, J=6.1 Hz, 2H), 7.58-7.44 (m, 7H), 7.44-7.38 (m, 2H), 7.37-7.31 (m, 2H), 7.30-7.19 (m, 5H), 6.83-6.77 (m, 2H), 6.70-6.64 (m, 2H), 5.62 (ddd, J=8.3, 5.4, 4.0 Hz, 2H, H-2″, H-7′″), 5.51 (s, 1H, Benzylidine), 5.39 (ddd, J=9.2, 6.6, 2.6 Hz, 1H, H-3′″), 5.35-5.31 (m, 2H, H-3′), 5.27 (d, J=8.3 Hz, 1H, H-1″), 5.18 (dt, J=7.3, 6.0 Hz, 1H, H-5′), 5.13 (dd, J=3.0, 1.1 Hz, 1H), 4.94 (d, J=8.5 Hz, 1H, H-1), 4.77 (d, J=4.0 Hz, 1H, H-1′), 4.71 (dd, J=10.6, 8.8 Hz, 1H, CH₂), 4.52 (dd, J=9.6, 1.5 Hz, 1H, H-6′″), 4.47-4.40 (m, 2H, CH₂), 4.40-4.22 (m, 7H, H-2, CH₂), 4.07-3.98 (m, 2H, CH₂), 3.92 (ddd, J=13.4, 11.2, 3.5 Hz, 1H, H-4′″), 3.84 (dd, J=11.5, 1.7 Hz, 1H, CH₂), 3.76 (s, 3H, CO₂Me), 3.74-3.66 (m, 2H, CH₂CH₂N₃), 3.63 (q, J=1.5 Hz, 1H), 3.59 (dd, J=11.2, 9.6 Hz, 1H, H-5′″), 3.51 (d, J=3.8 Hz, 1H, H-2′), 3.49 (s, 3H, Me-PMB), 3.32 (ddd, J=10.7, 8.1, 3.5 Hz, 1H, CH₂CH₂N₃), 3.24-3.14 (m, 2H, CH₂CH₂N₃), 3.06 (ddd, J=13.2, 5.2, 3.5 Hz, 1H, CH₂CH₂N₃), 2.82 (dd, J=12.0, 3.5 Hz, 1H, H-3′″), 2.52 (s, 3H, NAc), 2.13 (s, 3H, OAc), 1.92 (s, 4H, OAc, H-3′″), 1.85-1.80 (m, 9H, OAc), 1.20 (s, 9H, OTBDPS), 0.73 (d, J=6.5 Hz, 3H, H-6″). ¹³C NMR (151 MHz, Chloroform-d) δ 171.93, 169.78, 169.53, 167.65, 164.51, 153.53, 137.65, 135.85, 135.70, 133.99, 132.75, 129.97, 129.87, 129.77, 129.61, 129.60, 129.36, 128.55, 128.01, 127.70, 127.68, 126.20, 113.38, 100.36 (C-1″), 99.29 (C-Benzylidine), 98.42 (C-2′″), 97.80 (C-1″), 97.58 (C-1), 75.65, 75.41 (C-4′″), 75.21, 74.98 (C-6′″), 73.43, 73.25, 73.08, 72.76, 72.53 (C-2′), 71.31 (C-2″), 71.22 (C-2′), 70.80 (C-3′), 69.81 (H-2″), 68.39 (C-3′″), 67.25, 66.99 (CH₂CH₂N₃), 64.95 (C-5′), 59.26, 56.32 (C-2), 53.39 (C-Me-PMB), 50.32 (CH₂CH₂N₃), 36.29 (C-3′″), 31.93, 29.71, 29.67, 29.37, 27.17 (C-TBDPS), 24.73 (C—NAc), 22.70, 21.17, 20.94, 20.64, 20.55 (5C-OAc), 14.77 (C-6′); [α]_{D}²⁸=−2.3 (c=0.02, CH2Cl2); R_{f}: 0.40 (20% Acetone:Toluene); HR-MALDI-TOF/MS (positive, SuperDHB matrix): m/z calcd for C₈₉H₉₉N₅NaO₃₂Si [M+Na]⁺ 1800.5940, found: 1800.5945.

### 2-Azidoethyl-(5-acetamido-3,5-dideoxy-D-glycero-α-D-galacto-2-nonulopyranosylonicacid)-(2→3)-b-D-galactopyranosyl-(1->4)[(1→3)-α-L-fucopyranosyl]-2-acetamido-2-deoxy-β-D-glucopyranoside (19)

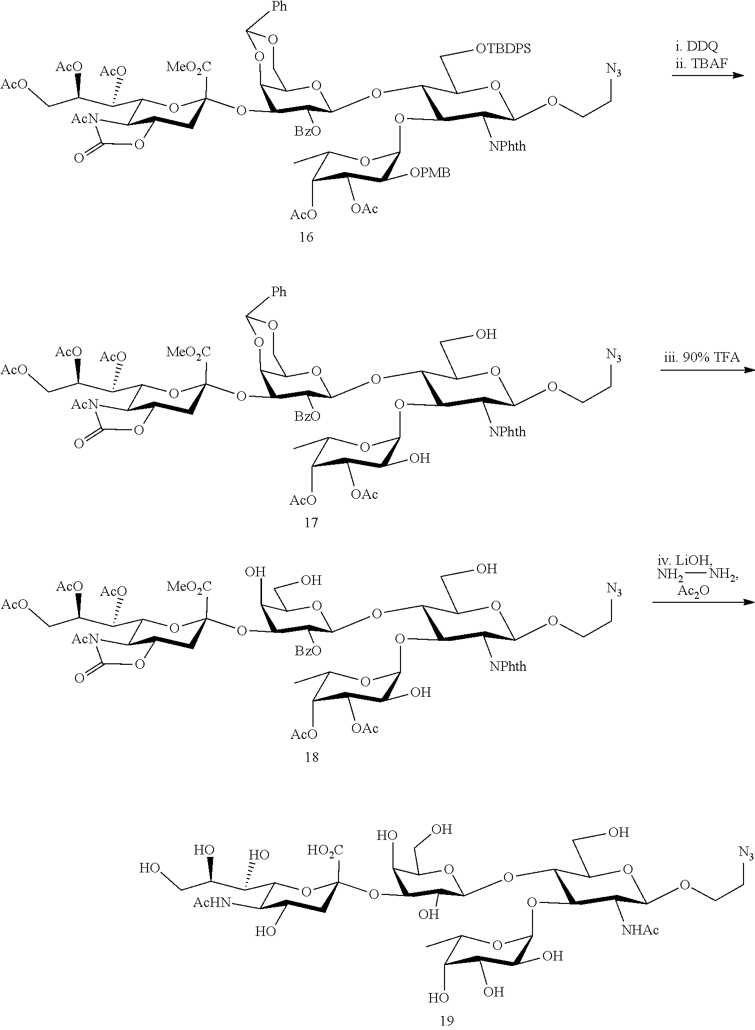

Compound 17. DDQ (123 mg, 0.542 mmol, 3.0 equiv.) was added to a stirred solution of protected 2-azido sLe^{x} 16 (320 mg, 0.180 mmol, 1.0 equiv.) in a mixture of CH₂Cl₂ and water (8.0+0.8 mL, 20:1) and the reaction mixture stirred until TLC showed complete consumption of the starting material. (˜5 h). The mixture was diluted with CH₂Cl₂, washed with sat. NaHCO₃ (twice), brine solution, and concentrated under reduced pressure. The residue was purified by flash-column chromatography on eluted on a silica gel with a EtOAc:hexanes systems to afford the desired product (260.0 mg, yield: 87%) as a white foam. The foamy compound (100.0 mg, 0.060, 1.0 equiv.) was dissolved in anhyd. DMF and the solution treated with TBAF (1M THF solution, 0.30 mmol, 2.0 equiv.) and acetic acid (18 mg, 0.30 mmol, 2.0 equiv.) simultaneously at ice-cooled temperature. The reaction mixture was stirred at ice cooled temperature for 30 min and then stirred at RT for ˜42 h. The mixture was diluted with EtOAc, washing with sat. NaHCO₃ followed by brine solution. The residue was purified by column chromatography on silica gel eluting with EtOAc:hexanes systems to afford the desired sLe^{x} diol 17 (150.0 mg, yield: 74%) as a white foam. R_{f}=0.30 (40% acetone:toluene); ¹H NMR (400 MHz, Chloroform-d) δ 8.18 (dd, J=8.0, 1.4 Hz, 2H), 7.76 (dd, J=5.4, 3.1 Hz, 2H), 7.64 (dd, J=5.5, 3.0 Hz, 2H), 7.61-7.54 (m, 1H), 7.48 (ddd, J=7.4, 4.4, 2.8 Hz, 4H), 7.33-7.18 (m, 4H), 5.80-5.70 (m, H-8′″), 5.49 (dd, J=9.6, 2.1 Hz, 1H, H-7′″), 5.41 (dd, J=10.1, 8.2 Hz, 1H, H-2″), 5.36 (s, 1H, Benzylidene), 5.07-5.00 (m, 3H, H-1, H-5′), 4.97-4.91 (m, 2H, H-1″), 4.71-4.52 (m, 5H, H-1′, H-9′″CH₂, H-6′CH₂), 4.48-4.38 (m, 2H), 4.24-4.17 (m, 1H, H-2), 4.17-4.10 (m, 1H, CH₂), 4.06 (dd, J=10.5, 8.4 Hz, 2H), 3.97 (dd, J=12.2, 9.5 Hz, 1H), 3.89 (d, J=3.9 Hz, 1H), 3.88-3.78 (m, 2H, CH₂CH₂N₃), 3.78-3.71 (m, 1H), 3.63 (s, 3H, CO₂Me), 3.61-3.50 (m, 3H), 3.48-3.38 (m, 3H, H-2′, CH₂CH₂N₃), 3.21 (ddt, J=11.2, 8.2, 3.8 Hz, 2H, CH₂CH₂N₃), 3.10-3.04 (m, 1H, CH₂CH₂N₃), 3.04-2.96 (m, 1H, H-3′″), 2.40 (s, 3H, NAc), 2.17 (s, 3H, OAc), 2.05 (s, 3H, OAc), 1.96 (s, 3H, OAc), 1.86 (s, 4H, OAc, H-3′″), 1.76 (s, 3H, OAc), 0.66 (d, J=6.4 Hz, 3H, H-6′). ¹³C NMR (100 MHz, cdcl₃) δ 172.68, 171.99, 170.85, 170.58, 170.21, 169.44, 168.95, 168.46, 164.70, 156.12, 153.33, 137.21, 134.13, 133.27, 131.51, 129.97, 129.77, 128.54, 128.23, 128.07, 125.68, 125.55, 123.53, 100.44 (C-1″), 99.91 (C-Benzylidene), 99.30 (C-1′), 97.77 (C-1), 96.81 (C-2′″), 77.41 (C-5), 77.29, 77.09, 76.77, 75.54, 75.01, 74.84, 74.79, 74.05, 73.03, 72.45, 72.28, 71.80, 70.97, 69.92, 69.26 (CH₂), 68.17 (CH₂CH₂N₃), 67.43 (CH₂), 66.57, 66.45 (C-2′), 65.50, 64.92 (CH₂), 59.56 (CH₂), 58.74, 56.17 (C-2), 53.19, 53.16, 50.30 (CH₂CH₂N₃), 36.91 (C-3′″CH₂), 24.55, 21.40, 20.91, 20.84, 20.73, 20.54, 14.83 (C-6′); R_{f}: 0.30 (40% Acetone:Toluene); HR-MALDI-TOF/MS (positive, SuperDHB matrix): m/z calcd for C₆₅H₇₃N₅NaO₃₁Si [M+Na]⁺ 1442.4187, found: 1442.4182.

Compound 18. A solution of benzylidene protected 2-azidoethyl sLe^{x} diol 17 (120.0 mg, 0.084 mmol, 1.0 equiv.) dissolved in 90% TFA and stirred at rt for 30 min. The reaction mixture was poured into sat. NaHCO₃ solution and extracted with CH₂Cl₂ followed by washing with brine. The solvent was then removed under reduced pressure and purified by column chromatography on silica gel eluting with EtOAc:hexanes systems to afford the desired product 18 (90.0 mg, yield: 80%) as a white foam. ¹H NMR (600 MHz, Chloroform-d) δ 8.25 (dd, J=8.2, 1.5 Hz, 2H), 7.83-7.78 (m, 2H), 7.69 (dd, J=5.5, 3.0 Hz, 2H), 7.64-7.56 (m, 1H), 7.50 (t, J=7.7 Hz, 2H), 5.73 (td, J=9.6, 2.7 Hz, 1H, H-8′″), 5.41-5.35 (m, 2H, H-2″, H-7′″), 5.18-5.09 (m, 3H, H-1, H-5′, H-4′), 5.04 (dd, J=10.5, 3.2 Hz, 1H, H-3′), 4.95 (d, J=8.2 Hz, 1H, H-1″), 4.62 (dd, J=11.9, 3.2 Hz, 2H, H-1′, H-9′″CH₂), 4.45 (ddd, J=19.8, 9.8, 2.8 Hz, 3H, H-3″, H-3), 4.22 (dd, J=10.8, 8.4 Hz, 1H, H-2), 4.09 (dd, J=11.2, 8.1 Hz, 1H, H-4), 4.04-3.96 (m, 2H, H-6″CH₂, H-6CH₂), 3.96-3.91 (m, 3H, H-9′″CH₂, H-6″CH₂, H-2′), 4.91-3.86 (m, 1H, CH₂CH₂N₃), 3.86-3.75 (m, 2H, H-4′″, H-6CH₂), 3.79 (s, 3H, CO₂Me), 3.66-3.60 (m, 2H, H-5″, H-6′″), 3.60-3.52 (m, 3H, H-2′, H-4″, H-5′″), 3.47 (ddd, J=11.2, 8.2, 3.3 Hz, 1H, CH₂CH₂N₃), 3.34 (s, 1H, OH), 3.29-3.22 (m, 2H, H-5, CH₂CH₂N₃), 3.08 (ddd, J=13.3, 5.1, 3.3 Hz, 1H, CH₂CH₂N₃), 2.89 (dd, J=11.9, 3.3 Hz, 1H, H-3′″), 2.41 (s, 3H, NAc), 2.16 (m, 3H, OAc), 2.08 (m, 3H, OAc), 2.04-1.99 (m, 4H, OAc, H-3′″), 1.96 (s, 3H, OAc), 1.46 (s, 3H, OAc), 1.23 (m, 3H, H-6′). ¹³C NMR (151 MHz, Chloroform-d) δ 172.68 (C-9′″ OAc), 171.78 (C—NAc), 170.55, 170.48, 168.66 (C—CO₂Me), 164.83 (C-OBz), 153.39, 133.33, 130.28, 129.70, 128.51, 100.21 (C-1″), 99.00 (C-1′), 97.96 (C-1′), 97.24 (C-2′″), 75.61 (C-5), 74.98 (C-4″), 74.86 (C-4′″), 74.43, 73.75 (C-3), 73.68, 73.25 (C-4), 71.97 (C-2″), 71.91, 71.32, 70.67 (C-4′), 68.33 (CH₂CH₂N₃), 67.65, 67.50 (C-3′), 66.36, 65.22 (C-5′), 64.82 (C-9′″CH₂), 62.77, 59.74, 58.61, 56.11 (C-2), 53.33, 50.30 (CH₂CH₂N₃), 36.12 (C-3′″CH₂), 24.59, 21.31, 20.96, 20.88, 20.63, 20.41, 15.75 (C-6′); R_{f}=0.25 (50% Acetone:Toluene); HR-MALDI-TOF/MS (positive, SuperDHB matrix): m/z calcd for C₅₈H₆₉N₅NaO₃₁ [M+Na]⁺ 1354.3875, found: 1354.3878.

Compound 19. The sLe^{x} tetrol 18 (43.0 mg, 0.032 mmol, 1.0 equiv.) was treated with lithium hydroxide monohydrate (16.7 mg, 0.396 mmol, 12.0 equiv.; 1M solution) in methanol for 1 h. The reaction mixture was then neutralized with Amberlist H⁺ and concentrated under reduced pressure. A solution of the aforementioned crude compound in ethanol (4.0 mL) and water (1.0 mL) was treated with hydrazine hydrate (0.2 mL) at 100′C for 15 h. The reaction mixture was concentrated under reduced pressure, azeotrope with ethanol (×3) and dried for 12 hrs. Finally, acetic anhydride (50 μL) was added to a stirred solution of the crude in methanol (2.0 mL) and stirred for 45 min. The reaction mixture was then concentrated under reduced pressure and dried for 6 h before subjecting to HPLC purification. (9.0 mg, 35% overall 3 steps). ¹H NMR (600 MHz, Deuterium Oxide) δ 5.02 (d, J=4.0 Hz, 1H, 1′-H), 4.79-4.71 (m, 1H, 5′-H), 4.52 (d, J=8.4 Hz, 1H, 1-H), 4.44 (d, J=7.8 Hz, 1H, 1″-H), 4.04 (dd, J=9.8, 3.2 Hz, 1H, 3″-H), 3.99-3.90 (m, 2H, CH₂CH₂N₃, H—CH₂), 3.90-3.83 (m, 3H, H-2, H-4″), 3.80 (m, 6H, H-3, H-3′, H-5′″, H—CH₂), 3.72-3.63 (m, 4H, CH₂CH₂N₃, H-4′″), 3.63-3.55 (m, 4H, H-2′, H-4′), 3.54-3.48 (m, 3H, H-4), 3.45 (dd, J=9.8, 7.9 Hz, 1H, H-2″), 3.43-3.37 (m, 1H, CH₂CH₂N₃), 3.37-3.30 (m, 1H, CH₂CH₂N₃), 2.68 (dd, J=12.7, 4.6 Hz, 1H, 3′″-CH₂), 1.95 (d, J=0.8 Hz, 6H, 2 of NHCOCH₃), 1.78 (t, J=12.3 Hz, 1H, 3′″-CH₂), 1.08 (d, J=6.6 Hz, 3H, 6′-H); ¹³C NMR (151 MHz, Deuterium Oxide) δ 174.97, 174.40, 172.37 (1′″-C), 101.53 (1″-C), 100.82 (1-C), 98.66 (2′″-C), 98.62 (1′-C), 75.54 (3″-C), 75.26, 74.78, 74.74, 73.33, 73.07 (4-C), 71.87, 71.22, 69.25, 69.18, 68.67 (CH₂CH₂N₃), 68.14, 67.69, 67.67, 67.18 (2-C), 66.64 (5′-C), 62.81 (CH₂), 61.33 (CH₂), 59.61 (CH₂), 55.68, 51.64, 50.36 (CH₂CH₂N₃), 39.33, 22.29, 22.04 (2C′s-CH₃CONH), 15.23 (6′-C); HR-MALDI-TOF/MS (positive, SuperDHB matrix): m/z calcd for C₃₃H₅₅N₅NaO₂₃ [M+Na]⁺ 912.3186, found: 912.3198.

### Characterization of G4 (1)

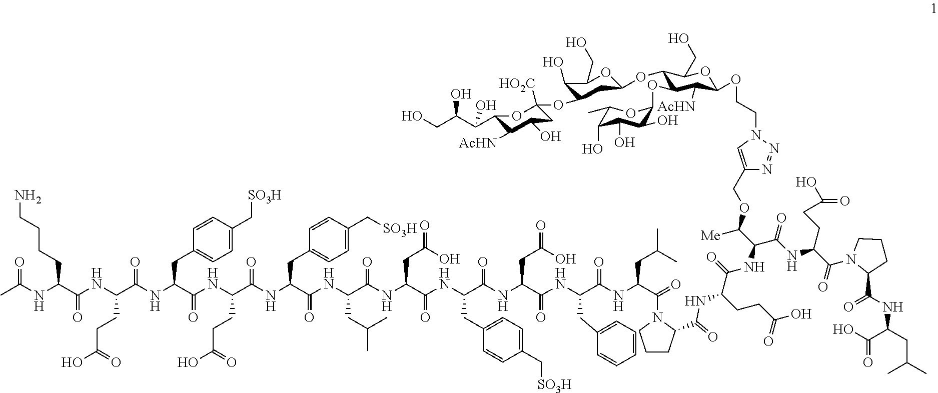

¹H NMR (600 MHz, Deuterium Oxide) δ 7.89 (s, 1H), 7.28-7.07 (m, 16H), 4.95 (d, J=4.1 Hz, 1H), 4.56-4.45 (m, 8H), 4.44-4.23 (m, 8H), 4.19 (dd, J=8.4, 4.3 Hz, 2H), 4.12 (ddd, J=21.9, 8.7, 6.3 Hz, 3H), 4.07-4.00 (m, 6H), 3.97 (d, J=3.2 Hz, 2H), 3.96-3.88 (m, 2H), 3.87-3.70 (m, 8H), 3.70-3.60 (m, 6H), 3.60-3.54 (m, 3H), 3.53-3.38 (m, 5H), 3.16-3.10 (m, 1H), 3.09-2.81 (m, 9H), 2.76-2.55 (m, 5H), 2.50-2.37 (in, J=7.5 Hz, 2H), 2.33 (s, 1H), 2.26-2.02 (m, 8H), 2.00-1.91 (m, 8H), 1.65-1.52 (m, 7H), 1.50 (d, J=10.3 Hz, 2H), 1.41 (d, J=11.5 Hz, 2H), 1.31 (dq, J=14.3, 7.1 Hz, 2H), 1.15 (dd, J=6.6, 2.9 Hz, 3H), 1.07 (t, J=5.6 Hz, 3H), 0.87-0.75 (m, 17H).

### Results:

### Compound Design and Strategy

Among a library of glycosulfopeptide analogues of the N-terminal domain of PSGL-1, previously screened for binding affinity to P-, L-, and E-selectin (44), GSnP-6 was identified as a low nanomolar orthosteric inhibitor of P-selectin. Computational simulations predicted that a number of key structural residues were most likely responsible for P-selectin/GSnP-6 interactions and prompted this investigation to explore the design of structurally simplified analogues of GSnP-6 as P-selectin antagonists. Consistent with experimental data (59), prior computational studies ranked the relative contributions to P-selectin binding with Fuc≈total sulfate >Neu5Ac, with the Thomsen (T) antigen disaccharide (i.e., Galβ1-3GalNAcα1) associated with minimal binding contributions (44). These observations provided a basis for replacing Galβ1-3GalNAcα1 with a linker between the peptide and sLe^{x} moiety. Critical design requirements for the linker included: (i) a geometry limited to the space occupied by the disaccharide; (ii) a synthetic route orthogonal to the synthesis of sLe^{x} and the peptide such that each building block could be produced independently and converge at the last step; (iii) associated functional groups that would not participate in additional, undesired binding events; and (iv) resistance to cleavage under chemical and biological conditions. To achieve these criteria, linkers attainable through click cycloaddition were examined (60), where sLe^{x} and the peptide bear azido (—N₃) or alkynyl (—RCC—R/H) groups, respectively. Two classes of triazole linkers (1-8) based on copper (CuAAC) and strain-promoted (SPAAC) azide-alkyne cycloaddition were designed.

### Theoretical Prediction of Linker Properties

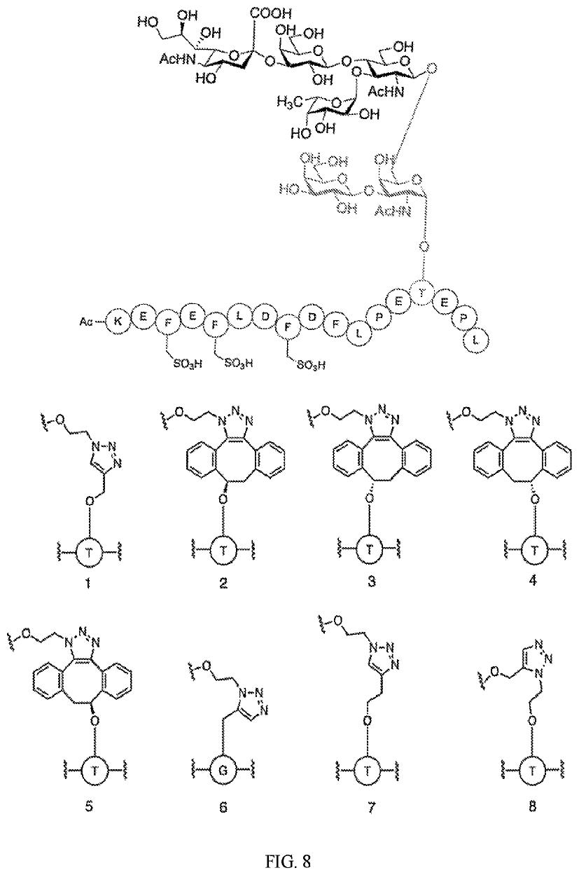

Computational simulations were used to model a subset of structurally simplified glycosulfopeptide variants possessing triazole linkers (1-8) (FIG. 8). The synthesis of multiple GSnP-6 analogues that possess a variety of stereospecific glycosidic linkages demands sophisticated schemes, considerable time, and large quantities of reagents, particularly for candidates that do not share intermediates. Targeted screening a priori using computational methods provides several advantages including: (i) hypothesis testing by evaluating the free energy of compound binding to P-selectin; (ii) elucidating structural insights into entropic and enthalpic components underlying the binding interactions; (iii) guidance to synthetic efforts (vide infra) by identifying a lead; and (iv) reducing time and cost towards identifying an optimal lead candidate.

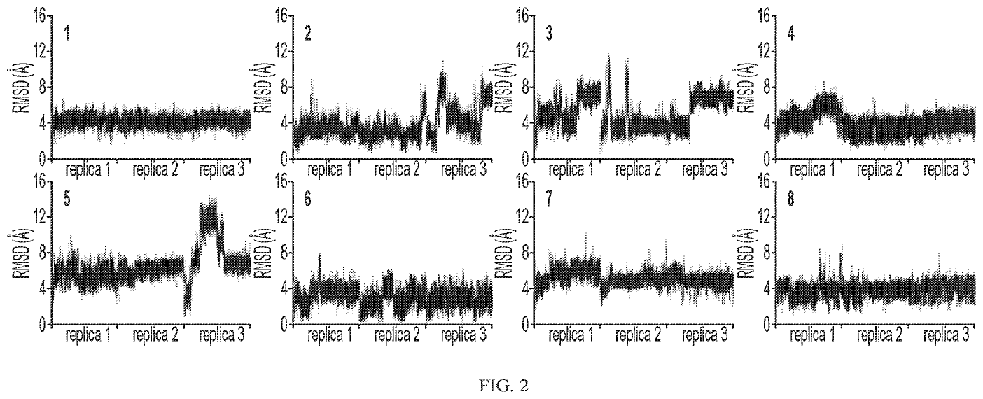

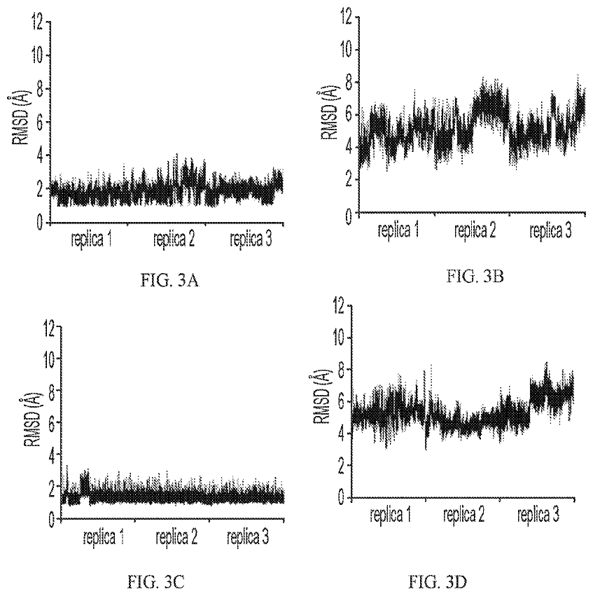

Molecular dynamics (MD) simulations were initially used to validate structural features critical to P-selectin binding. The positional root-mean-squared deviations (RMSD) of the O-linked threonine over the course of the MD simulation indicated that the structure was stable in the binding site of P-selectin (FIG. 2). Moreover, the glycan and the second sulfonated Phe (F48), which are responsible for high affinity P-selectin binding (44), displayed less positional variation than other residues in the peptide despite replacement of the T antigen disaccharide, consistent with stable interactions with P-selectin (FIG. 3). Key hydrogen bonds and salt bridges, as defined by the molecular model of P-selectin to GSnP-6 (44), were observed during the MD simulations (Table 3). These results confirm that compound 1 demonstrates stability with P-selectin comparable to GSnP-6. Molecular mechanics-generalized born solvent-accessible surface area (MM/GBSA) calculations using the GB₁^{OBC} model with an internal dielectric constant value (εᵢₙₜ) of 4.0 showed that the order of relative contributions of the glycan and sulfonated Phe to P-selectin binding were maintained, where Fuc≈total SO₃⁻>Neu5Ac, providing further validation of likely favorable binding interactions (Table 4). Due to the structural differences between the triazole moiety of 1 and the replaced disaccharide, MM/GBSA calculations were followed by a determination of the entropic penalty for accurate representation of the overall binding free energy (61). Significantly, the rotatable bonds of the linker introduce movements within the binding pocket that can alter the overall binding energy as a result of entropic differences, with binding of more flexible ligands less favored than more rigid ligands appropriately positioned within the binding domain.

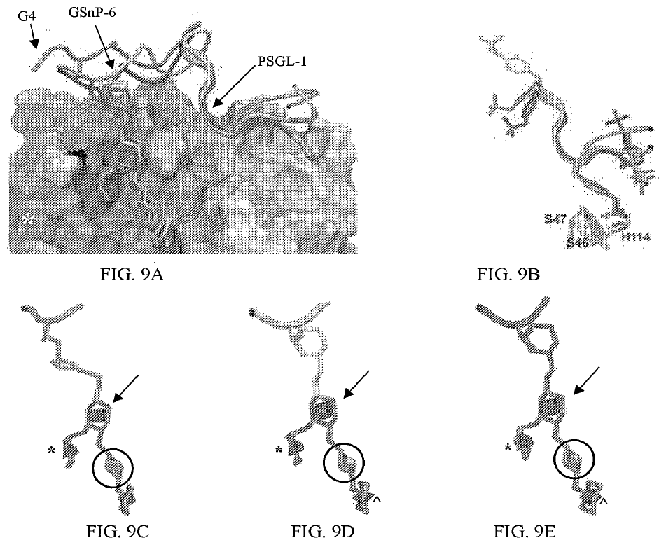

In this approach, configurational (−TΔS_{RTV}) entropic changes associated with rotations, translations, and vibrations, as well as conformational (−TΔS_{q}^{c}) entropic changes due to glycosidic torsion distributions were accounted for. Specifically, it was found that extrapolating the configurational entropic penalty at the infinite simulation time using the Quasi-Harmonic (QH) approximation provided reproducible means to estimate entropic penalties (−TΔS_{RTV}) (Table 1) (54, 55). Furthermore, a Karplus-Kuschick method (56) was used to calculate the conformational entropic penalties (−TΔS_{q}^{c}) by analysis of the covariance matrix of the torsional distributions relative to the unbound state. The overall binding free energies were calculated with convergence of the total entropic penalties, which predicted that candidate 1 retained binding energy to P-selectin comparable to GSnP-6. Synthetic considerations were then applied to support in silico results. Given the requirement to control stereospecificity of 5-8 using SPAAC, preference to CuAAC series 1-4 led us to select the candidate predicted to provide the most favorable binding (1), termed G4, as the lead candidate (FIG. 9A-9E).

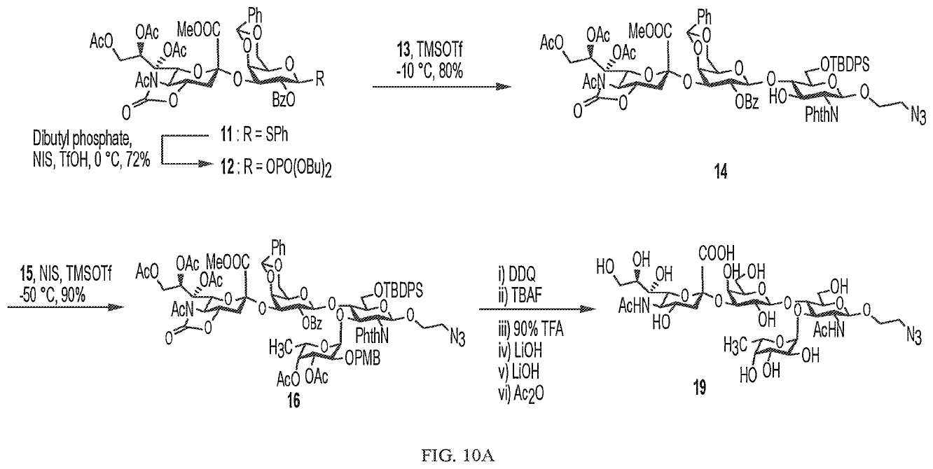

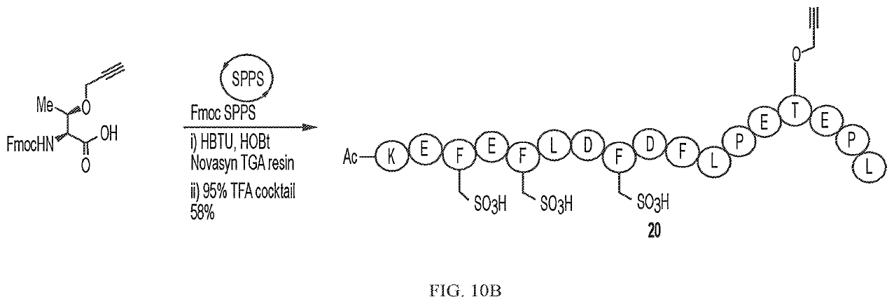

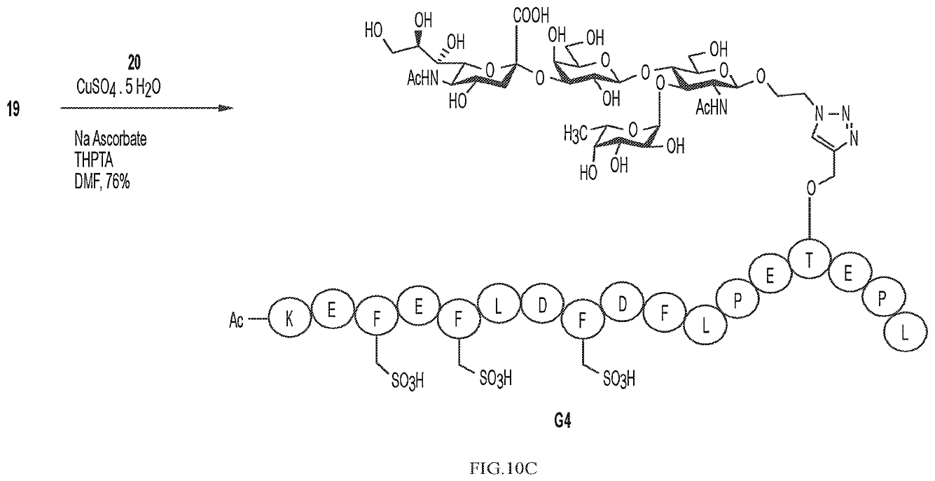

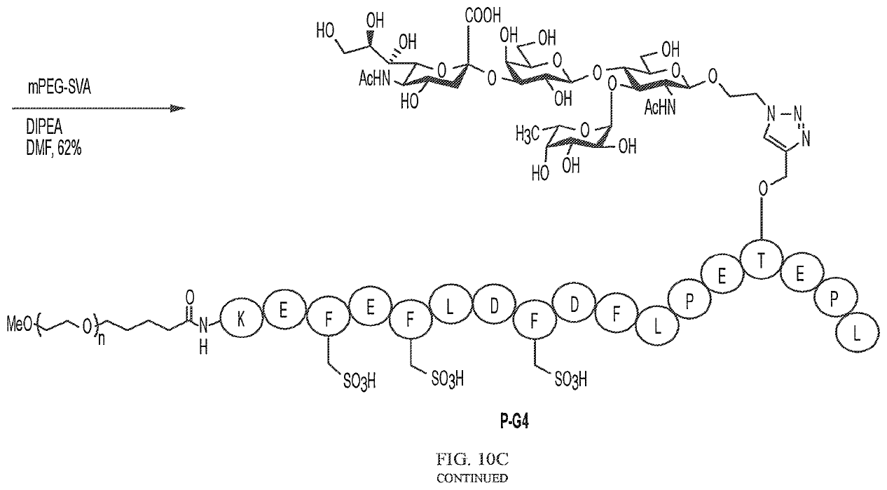

Synthesis of G4 In this retrosynthetic design, a route to G4 (1) was devised that would enable downstream coupling of azido sLe^{x} (19) to the propargyl peptide (20) mediated by CuAAC (FIG. 10A-10C). The propargyl peptide was initially synthesized using solid phase peptide synthesis (SPPS). O-propargylation of Fmoc-protected threonine was executed as previously described (57). A coupling strategy using HBTU incorporated the Fmoc O-propargyl threonine to the resin-bound peptide backbone with deprotection steps conducted in 20% piperidine. Notably, the propargyl group was preserved during both SPPS and the final cleavage step using 95% TFA/H₂O/TIPS, to afford the propargyl peptide 20 in 58% yield.

To date, only one account of the synthesis of sialyl Lewis^{X} bearing a 2-azidoethyl linker has been reported by Wu and co-workers (62). This approach utilizes bacterial glycosyltransferases and respective nucleotide sugars to achieve successful sialylation and fucosylation to a chemically synthesized lactosamine bearing a 2-azidoethyl linker, yielding less than 20 mg of the sLe^{x} azide (62). Due to limitations in cost, scalability, and variability associated with chemoenzymatic synthesis (63, 64), a total chemical synthesis of sLe^{x} azide was pursued (19). Several key challenges were initially identified. Given the structural complexity underlying stereospecific glycosidic linkages, strategic placement of protecting groups in sequence was imperative. In addition, these protecting groups must be removed without affecting the azide functional group of the linker. In previous attempts (65), sLe^{x} was synthesized using benzyl protected glycan intermediates that served as armed donors and acceptors. However, the requirement to remove benzyl groups using hydrogenolysis would reduce the azide functional group in this system as demonstrated in other azide bearing derivatives of sLe^{x} (66-68). Although the reduced amine could be re-converted to the azide, the added step and the potentially explosive nature of the triflic acid-mediated diazotransfer condition (69) renders this scheme unappealing for process optimization. It was found that the use of p-methoxybenzyl (PMB) protecting groups enables stereospecific glycosylation towards sLe^{x} with deprotection steps that were successful without compromising the azide functional group.

The synthesis of 19 began with preparation of monosaccharide building blocks 9, 10 13, and 15 equipped with appropriate protecting groups, which were synthesized according to previously reported protocols (70-74). A series of glycosylations generates a fully protected 2-azidoethyl sLe^{x} derivative (16) followed by deprotection steps to yield 19. A key disaccharide building block 11 featuring highly α-selective glycosidic linkage formation was synthesized using a 5-N, 4-O-oxazolidinone sialic acid phosphate donor in 75% yield (75). Subsequently, the disaccharide thioglycoside equipped with a benzoyl (Bz) group at the C-2 position enables direct β-linked glycosylation without observing aglycon transfer or orthoester formation (76). Subsequently, the preparation of 2-azidoethyl GlcNAc acceptor (13) was focused on bearing 2-N-phthalyl (Phth) and 6-O-tert-butyldiphenyl silyl (TBDPS) protecting groups (77). The selection of the N-Phth group, in particular, was important as it allows β-linked glycosylation of 2-azido ethanol and its removal remains orthogonal to the azide functional group (67). To synthesize trisaccharide 14, a [2+1] glycosylation between the disaccharide donor 11 and acceptor 13 was employed. In the initial attempt, glycosylation of acceptor 13 to disaccharide donor 11 using an NIS-TfOH promoter yielded only 30-40% of the trisaccharide product. Given the disarmed nature of the donor, switching to a more activated leaving group, such as a phosphate leaving group from the thioglycoside, was hypothesized to improve the reaction profile (75). Sialylgalactosyl phosphate 12 was synthesized from the corresponding thioglycoside derivative using dibutyl phosphate under NIS and TfOH activation in 72% yield (75). Subsequently, the [2+1] glycosylation between acceptor 11 and the disaccharide phosphate donor 12 was carried out using TMSOTf to afford selective β-(1→4) linked trisaccharide 14 in 85% yield. Regioselectivity was observed, yielding exclusively β-(1→4) glycosylation, as a result of the synergistic effect of the highly reactive sialylgalactose phosphate donor and the reduced reactivity of the 3-OH masked by the N-phthalimide protecting group (67). Subsequent α-(1-3) fucosylation of trisaccharide 14 under an NIS-TfOH activation system afforded the protected sLe^{x} azide derivative 16 in 90% yield (78). Notably, the partially disarmed nature of the donor equipped with a p-methoxybenzyl (PMB) group at C-2 and acetyl groups at C-3/C-4 prevents neighboring group participation at C-2 leading exclusively to α-(1-3) fucosylation (79). Finally, global deprotection steps were carried out in a sequential manner. The removal of PMB ethers was achieved under oxidative cleavage using 2,3-dichloro-5,6-dicyano-1,4-benzoquinone (DDQ), which was followed by removal of the silyl protecting group using tetra-butylammonium fluoride (TBAF). Subsequently, the benzylidene protecting group was hydrolyzed using 90% trifluoroacetic acid and saponification of the acetyl groups and methyl ester was successful using lithium hydroxide (LiOH). The Bz and 2-N-phthalyl groups were removed under hydrazine treatment at high temperatures. The amine was selectively acetylated using acetic anhydride in methanol to generate the target 2-azidoethyl sialyl Lewis^{x} (19). Compound 19 was generated from 16 in 17% yield. Using this approach, the synthesis of 2-azidoethyl sialyl Lewis^{x} was achieved from 6.0 gm of the initial sialic acid derivative (N-acetyl-5-N,4-O-oxazolidinone-thio sialoside, 9a), yielding 150 mg of 19.

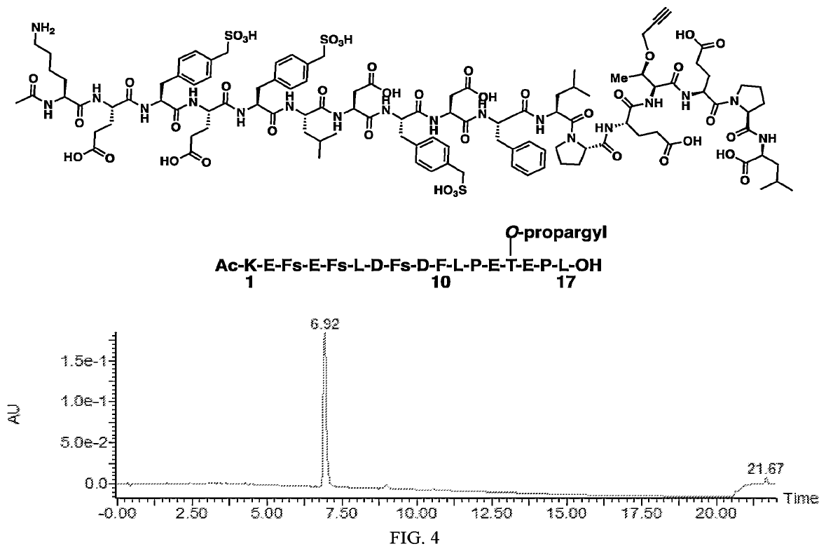

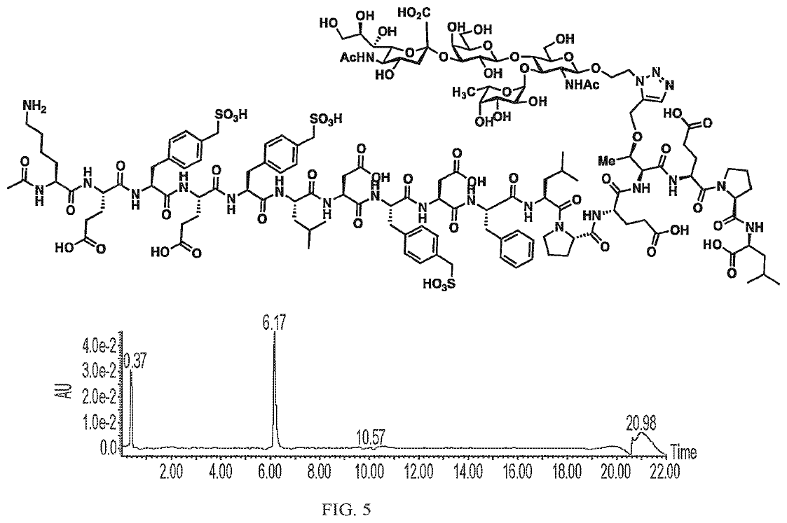

With successful syntheses of 19 and 20, the click reaction was initially performed using copper sulfate and ascorbic acid in DMF (80) affording G4 with a 51% yield. However, it was subsequently determined that use of tris(3-hydroxypropyl-triazolylmethyl)amine (THPTA) improved overall yield to 76% (81), which may reflect a better reaction profile provided by THPTA stabilization of the propargyl group (FIGS. 4-5) (82). Using a CuAAC reaction, a simple, site-specific method was demonstrated to incorporate a complex glycan into a charged peptide in free form.

### Synthesis of PEG-G4 (P-G4)

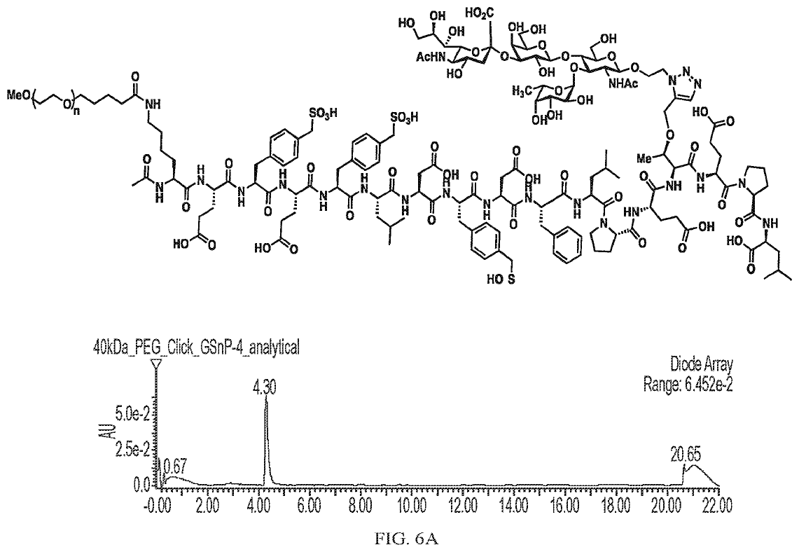

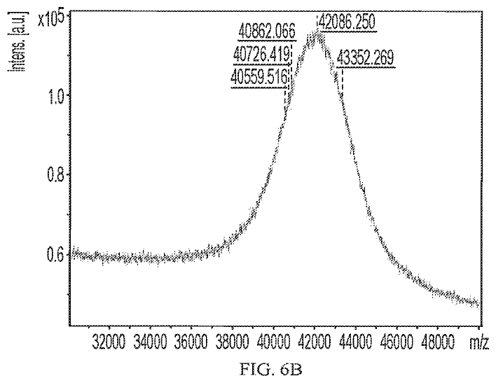

With an aim of extending circulating half-life (t_{1/2}), conjugation of polyethylene glycol (PEG) to the N-terminus of the glycosulfopeptide was explored (83). PEGylation under the activation of a p-nitrophenoxy or succinimidyl group in aqueous solution afforded only a scarce amount of product, presumably due to rapid hydrolysis of the PEGylation reagent. As such, the conjugation of G4 to 40 kDa methoxy polyethylene glycol succinimidyl valerate (mPEG-SVA) was explored in DMF. A 2:1 ratio of G4 to mPEG-SVA was used in the presence of Hünig's base and excess G4 was retrieved for subsequent rounds of PEGylation. PEG-G4 (P-G4) and unreacted G4 were isolated at different retention times using RP-HPLC to afford P-G4 in 64% yield following a single round of conjugation (FIGS. 6A-6B).

### G4 Demonstrates Nanomolar Affinity to P-Selectin

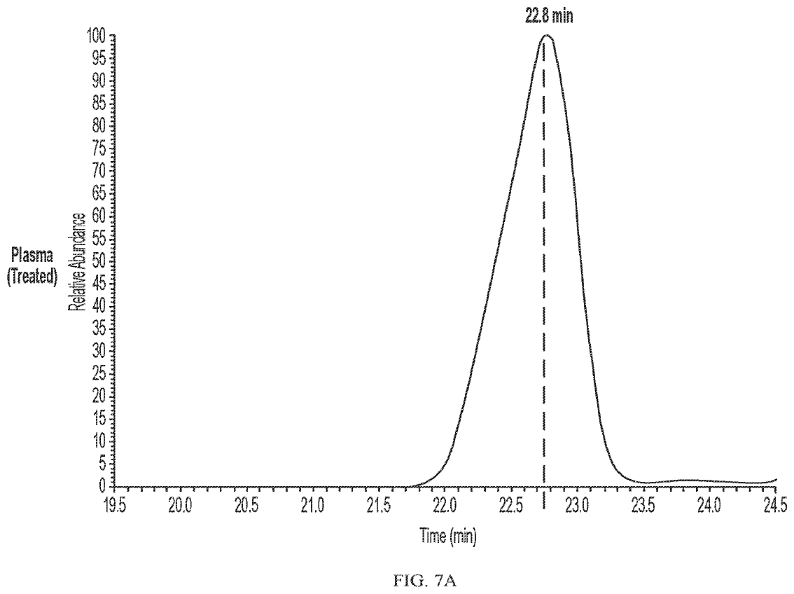

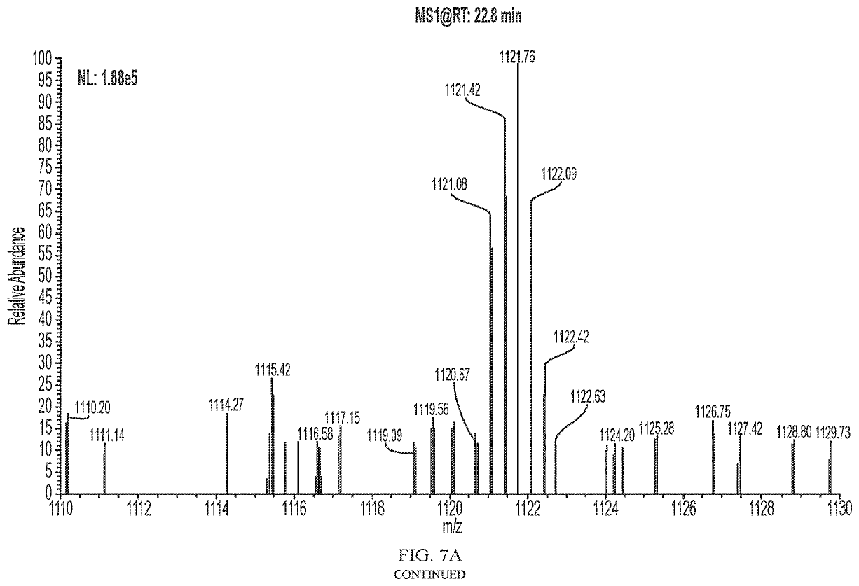

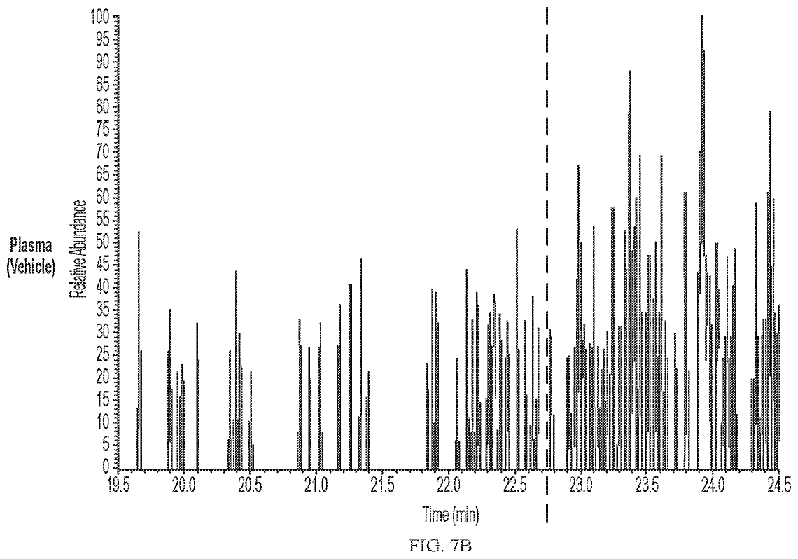

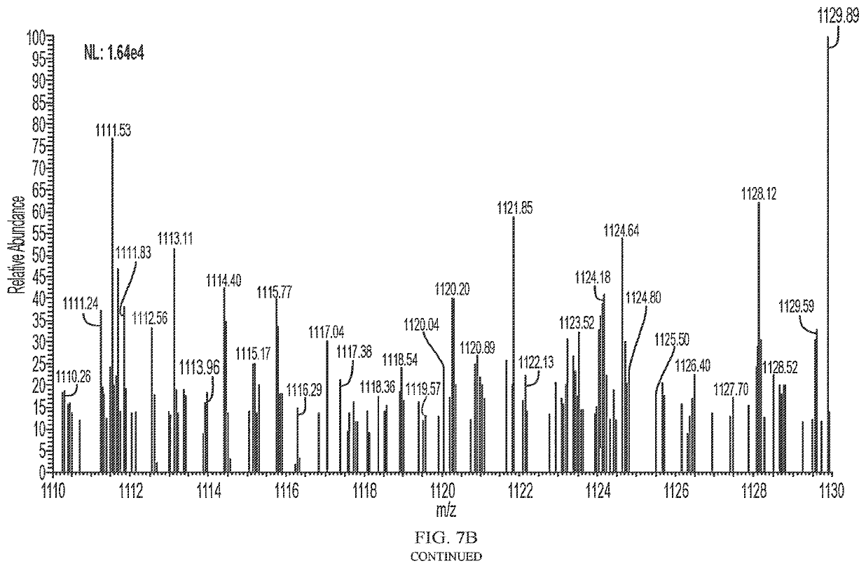

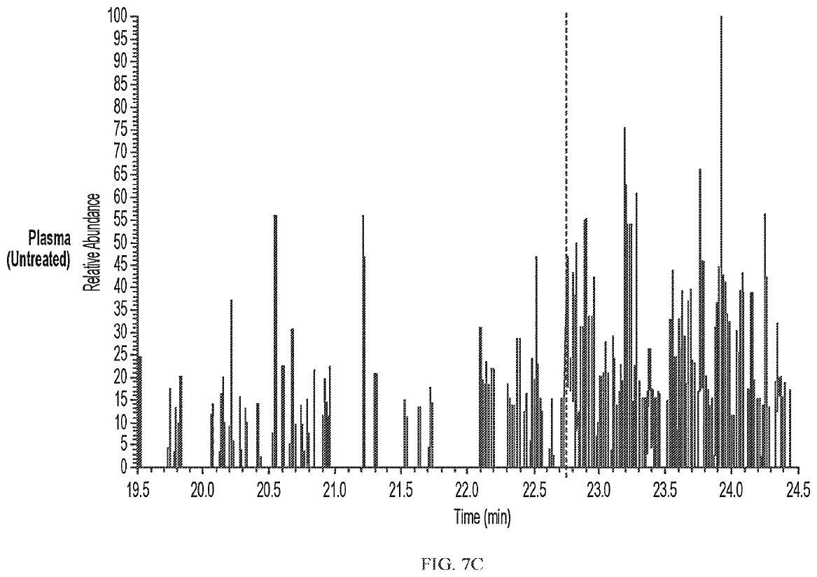

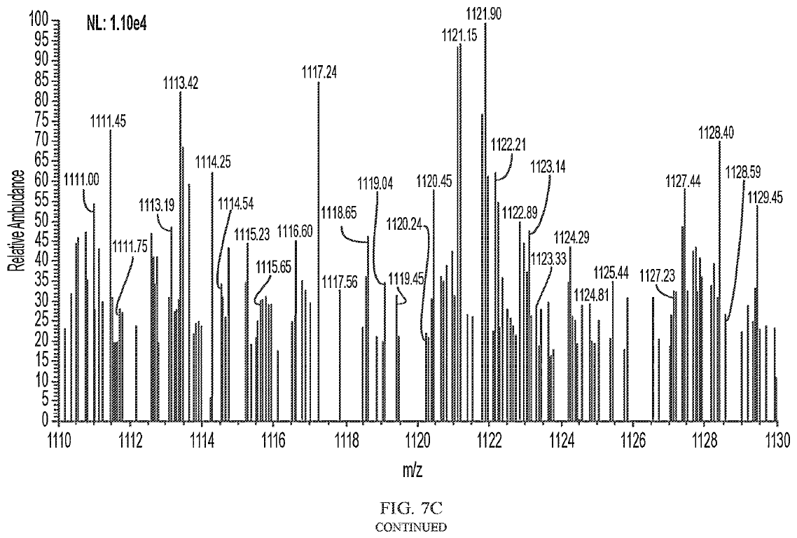

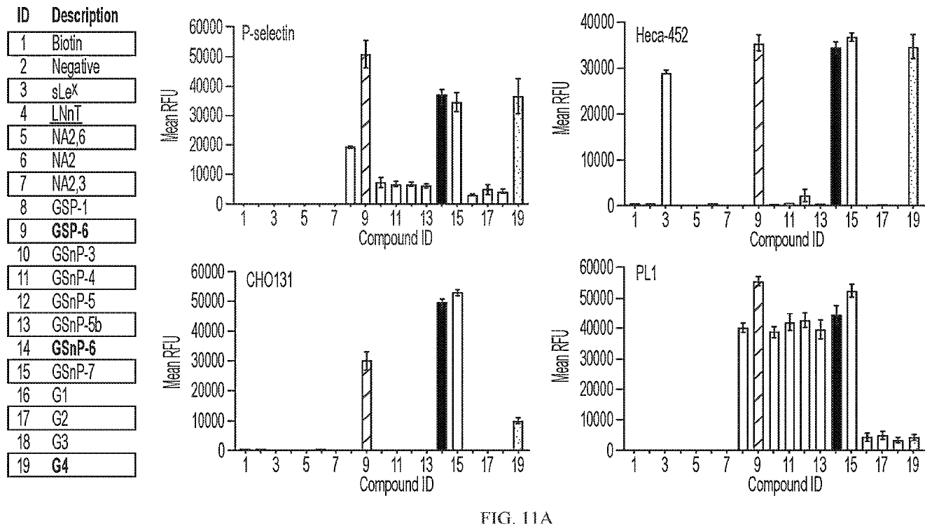

The binding affinity of G4 towards P-selectin was initially screened using microarray technology. G4 along with 11 additional structural variants and synthetic intermediates, as well as five glycan standards (sLe^{x}, NA2, NA2,3, NA2,6, asialobiantennary, asialotetra-saccharide) were printed on an NHS-activated glass slide (FIGS. 7A-7C). The slide was then incubated with a recombinant immunoglobulin (Ig) chimera of P-sel (5 μg mL⁻¹) or monoclonal antibodies, PL1, specific to the N-terminal PSGL-1 peptide sequence, CHO131, specific to the Core 2 O-glycan terminated with sLe^{X}, or HECA-452, specific to sLe^{x}. The array was then washed and incubated with Alexa-488-labelled anti-human IgG antibody (5 μg/mL). Similar to the native, N-terminal PSGL-1 sequence containing tyrosine sulfate (GSP-6) and GSnP-6, G4 strongly bound P-selectin (FIG. 11A). While HECA-452 strongly bound to GSP-6, GSnP-6 and G4, in contrast to GSP-6 or GSnP-6, neither PL1 or CHO131 bound to G4. Of note, the PL1 epitope spans residues 49-62, which includes the key sequence -L-P-E-T-E-P- (84). Thus, modification of the key T residue within the PL1 epitope alters PL1 binding, but not P-selectin binding. Binding of glycopeptide mimics was Ca₂⁺ dependent and inhibited by EDTA.

Dissociation constants (Kd) were determined using a Biacore binding SPR assay after initial capture of biotinylated G4 onto streptavidin-coated sensor chips followed by flow through of P-sel-Ig (0.4-100 nM). The dissociation constant for G4 to human P-selectin was 69 nM (FIG. 11B), as compared with the reported Kd of 73 nM for native PSGL-1 and 27 nM for GSnP-6 (44, 85).

### G4 and P-G4 Inhibit P-Selectin Binding to PSGL-1

Flow cytometry was used to evaluate the ability of G4 and P-G4 to block binding of P-selectin-Fc chimera to human and murine leukocytes. Recombinant species-specific P-selectin-Fc chimeras (3 μg/mL) were incubated with human or murine leukocytes in the presence of G4 or P-G4 (0-100 μM). Binding of P-selectin was detected with phycoerythrin (PE)-conjugated anti-Fc antibody and quantified as mean fluorescent intensity and plotted as percent inhibition. G4 and P-G4 inhibited P-selectin binding to both human and murine leukocytes in a dose-dependent manner. G4 inhibited P-selectin binding to human and murine leukocytes, including human monocytes (IC₅₀ 16 μM), human neutrophils (IC₅₀ 13 μM), murine monocytes (IC₅₀ 10 μM), and murine neutrophils (IC₅₀ 10 μM). In a comparable manner, P-G4 blocked P-selectin binding to human and murine leukocytes, including human monocytes (IC₅₀ 12 μM), human neutrophils (IC₅₀ 9 μM), murine monocytes (IC₅₀ 6 μM), and murine neutrophils (IC₅₀ 9 μM) (FIG. 11C).

### G4 and P-G4 Inhibit Platelet-Leukocyte Aggregation

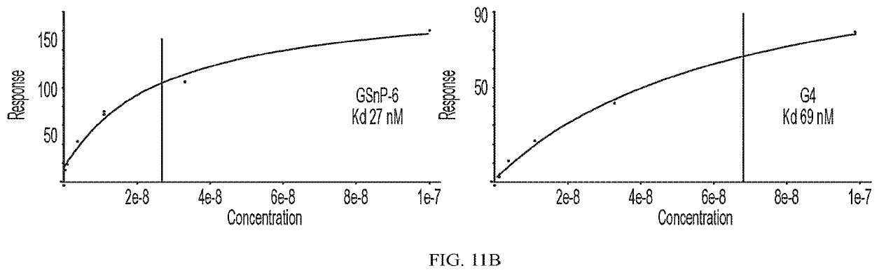

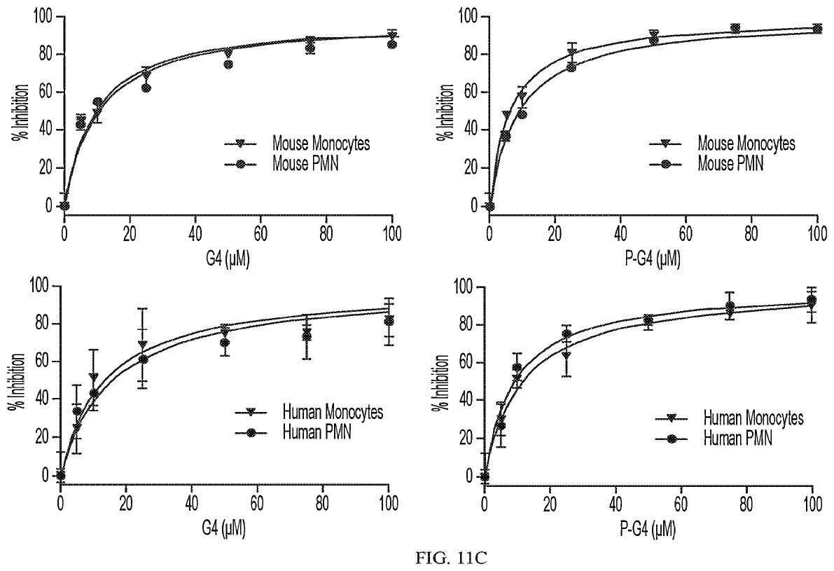

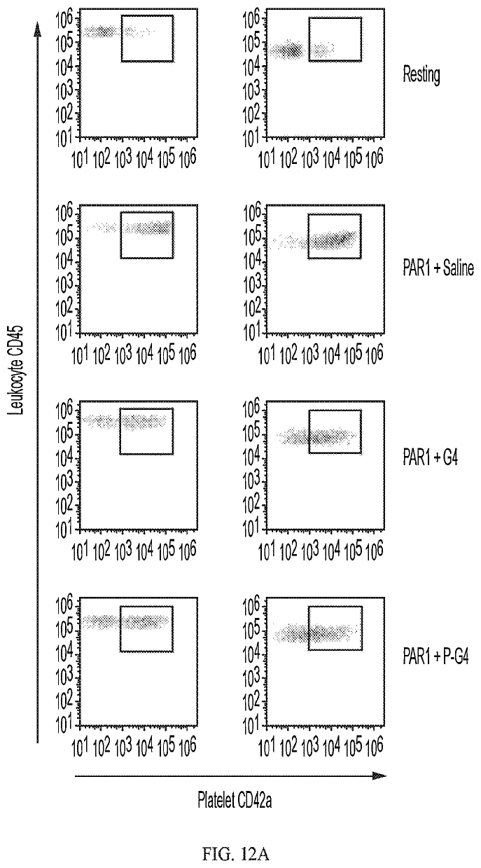

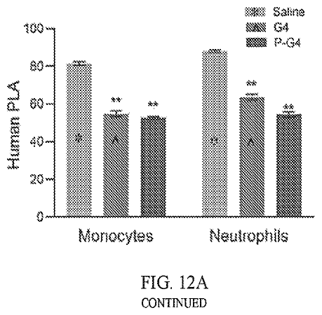

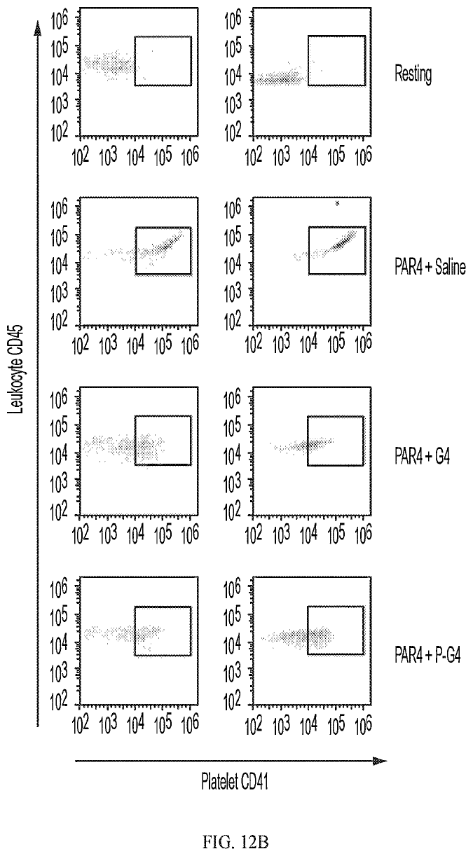

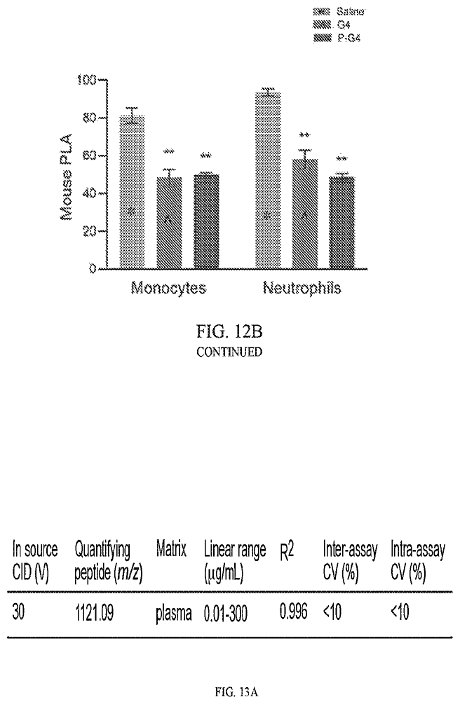

PSGL-1/P-selectin binding are responsible for the generation of platelet-leukocyte aggregates, which leads to the release of neutrophil extracellular traps and other procoagulant factors, as well as the infiltration of leukocytes (86, 87). The ability of G4 and P-G4 to inhibit platelet-leukocyte aggregation in human and murine blood was evaluated using flow cytometry (FIG. 12A,B). Platelet P-selectin expression was induced by exposure to mouse PAR₄-activating peptide or human PAR₁-activating peptide and dosed with 120 μM of G4 or P-G4. G4 inhibited 41% of murine platelet-monocyte and 38% of platelet-neutrophil aggregates, as well as 33% of human platelet-monocyte and 28% of platelet-neutrophil aggregates. Likewise, P-G4 reduced murine platelet-monocyte and platelet-neutrophil aggregates by 39% and 48%, respectively, and human platelet-monocyte and platelet-neutrophil aggregates by 35% and 38%, respectively.

### Pharmacokinetics of G4 and P-G4

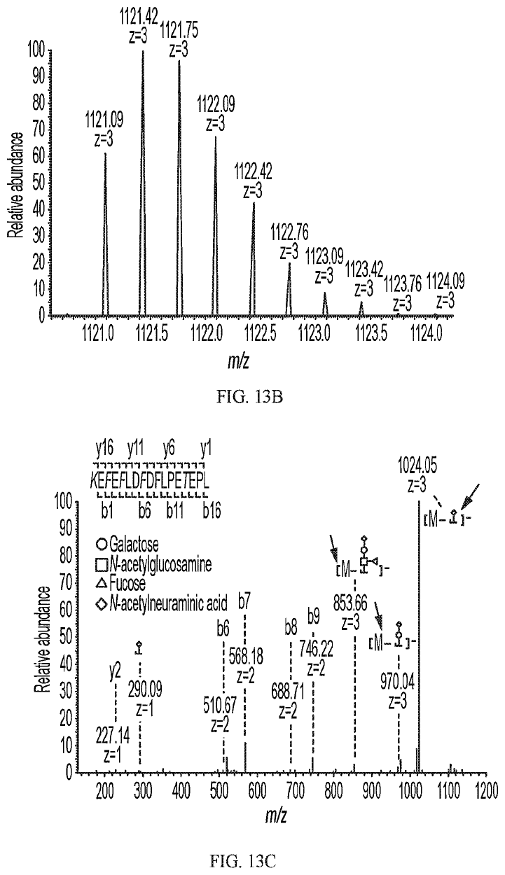

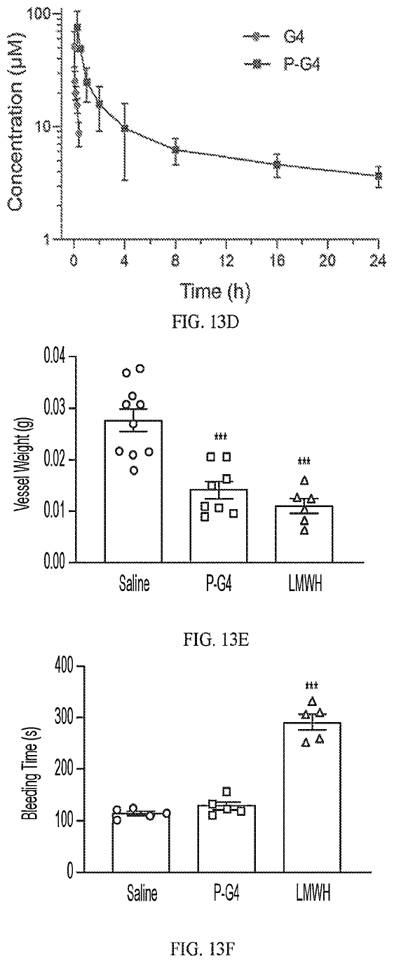

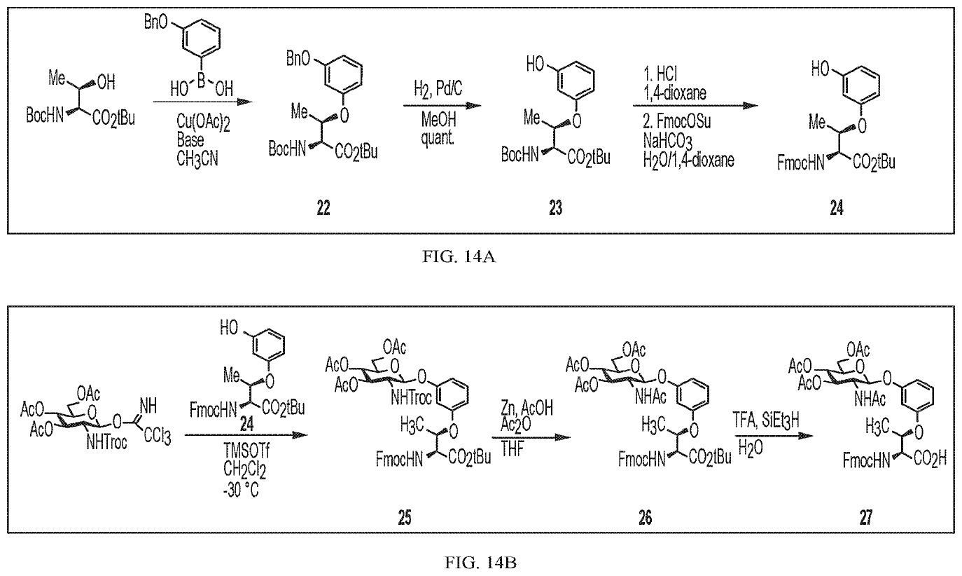

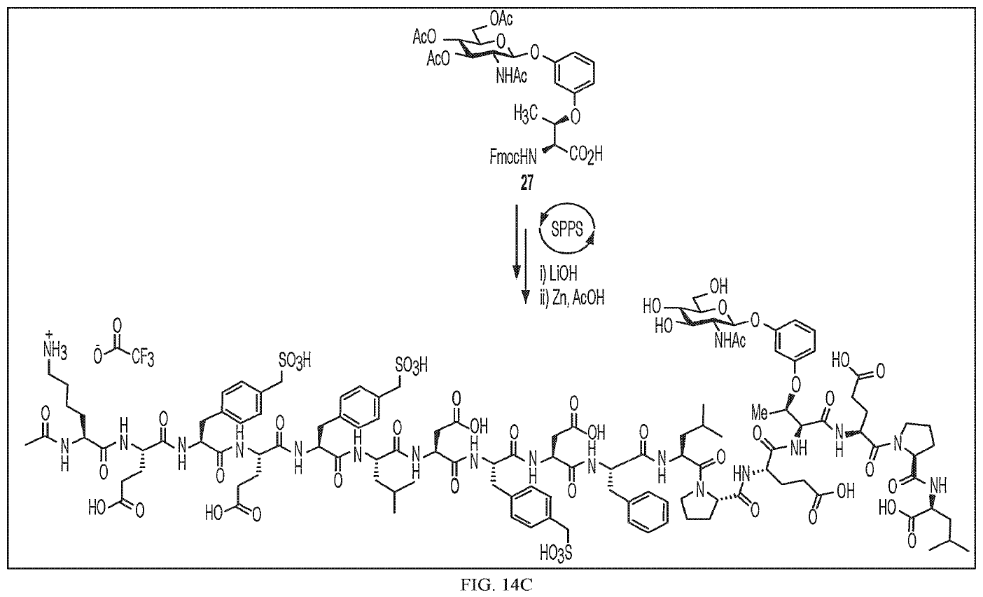

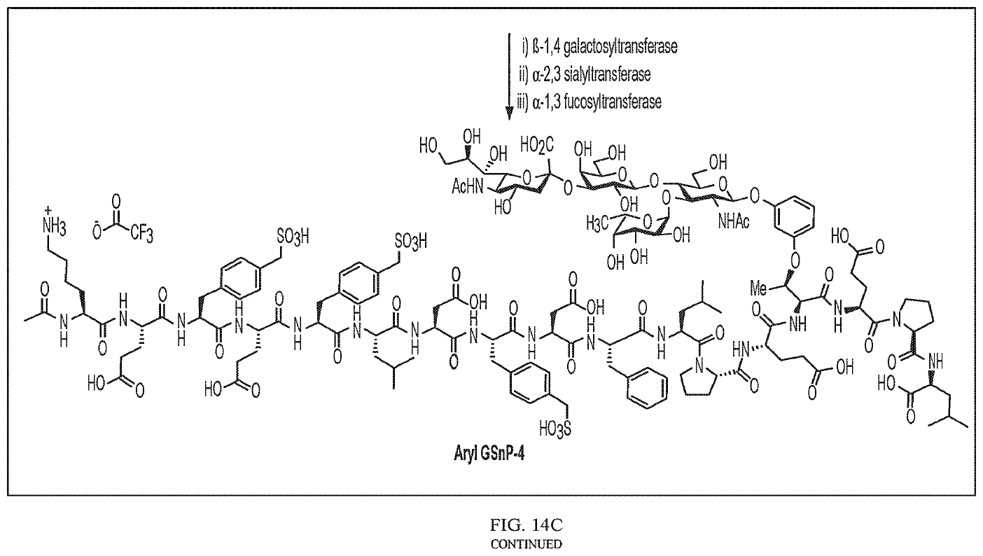

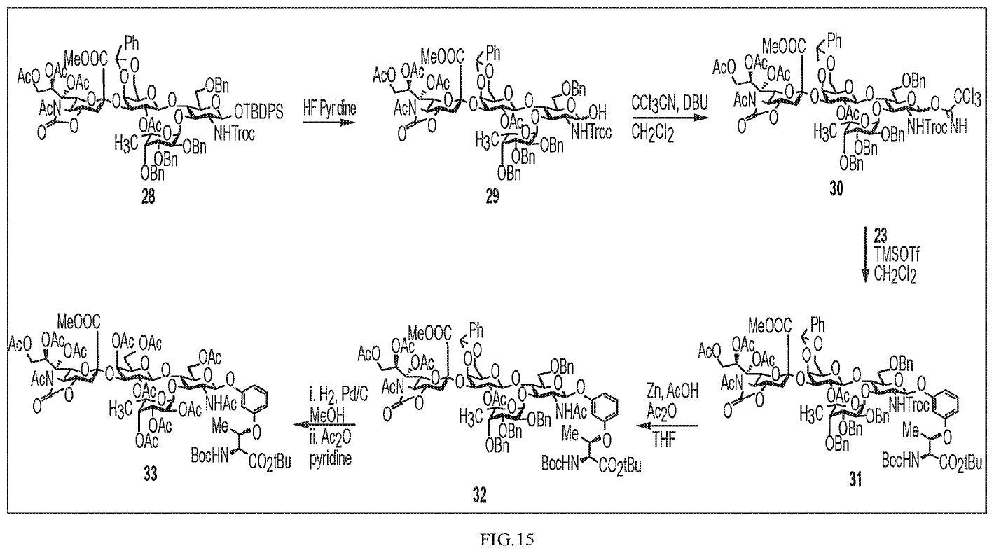

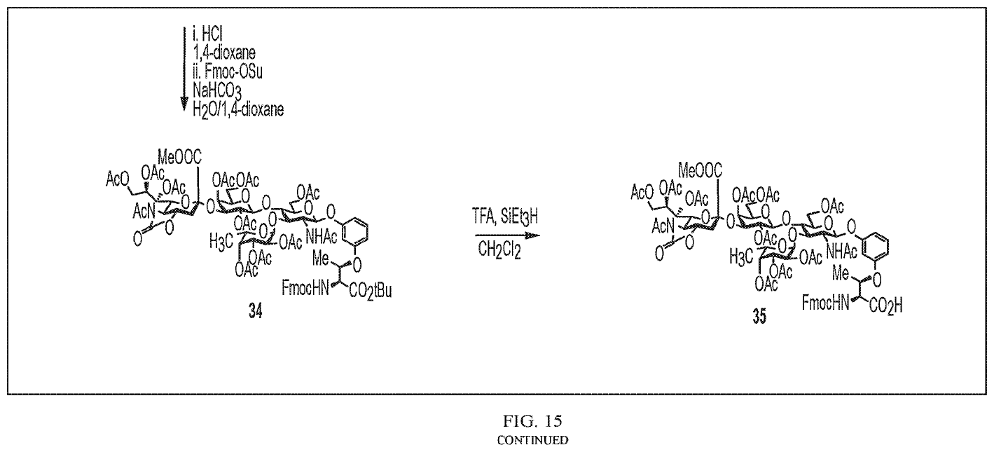

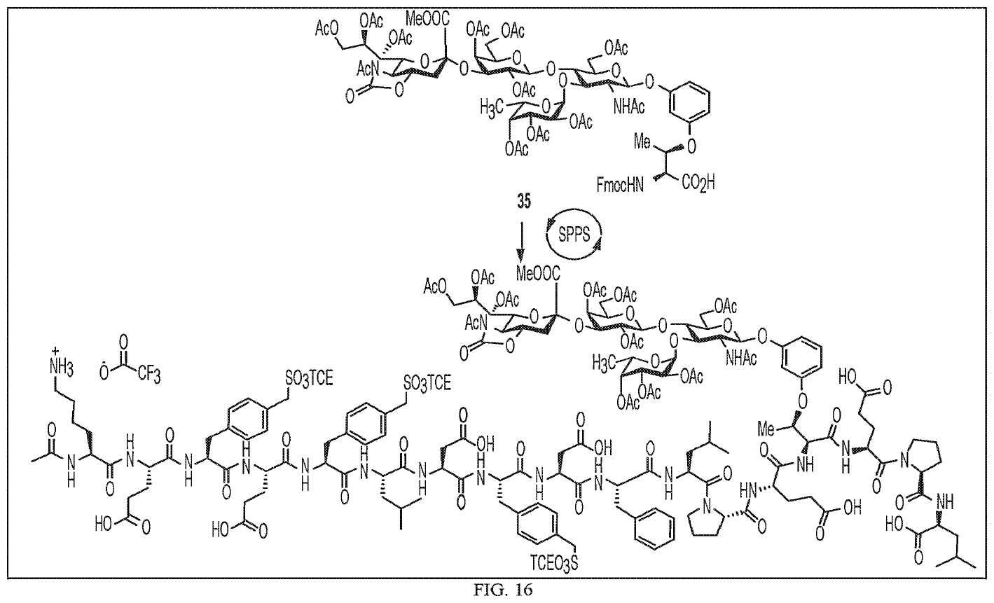

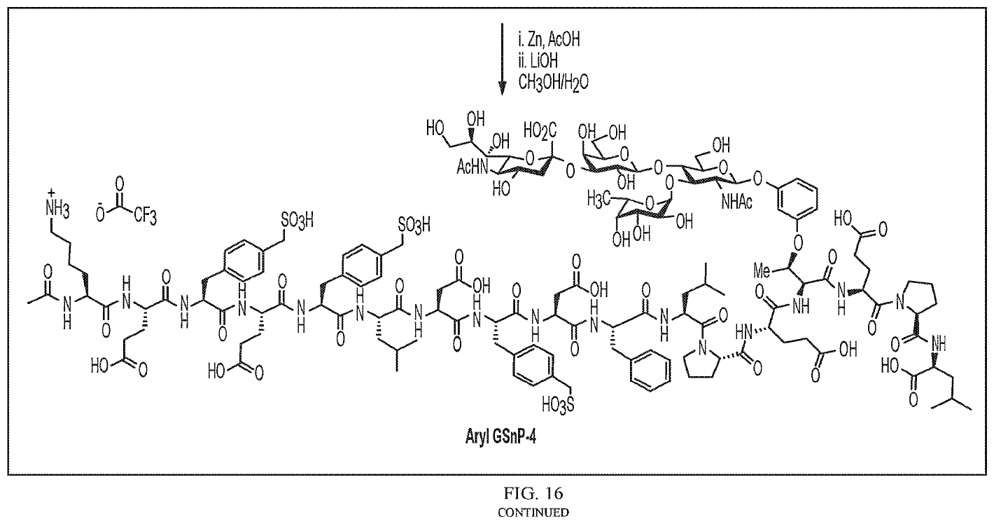

Nanoflow hydrophilic interaction liquid chromatography (HILIC)-electrospray ionization-mass spectrometry (ESI-MS) was used to evaluate chromatographic behavior of G4 and obtain distinguishing structural information. Stepped higher-energy collisional dissociation (HCD) was employed to generate product ions of the constituent parts of G4 in the MS/MS spectra, which was used along with column retention time as a signature profile for facile matching in subsequent samples (FIG. 13A). Structural identification was followed by quantification of G4 and P-G4 in mouse plasma. To facilitate the analysis of P-G4, the application of in-source fragmentation of P-G4 was investigated and a de-PEGylated ion (m/z 1121.09) as a reliable quantifying peptide was identified (FIG. 13B,C). Unlike enzymatic digestion, which lengthens sample preparation and data analysis, source fragmentation of P-G4 allowed samples to be directly injected without additional off-line separation, and analyzed by monitoring a single, resolved peptide designated as a surrogate for quantifying P-G4. The quantifying peptide was unique to samples containing P-G4 and absent from vehicle controls and native plasma (FIGS. 7A-7C). Gradient elution of repeat injections of extracted G4 and P-G4 yielded highly reproducible retention times without co-elution of other overlapping matrix components.

A single dose of G4 or P-G4 was administered intravenously to mice and in vivo plasma concentrations were measured over time. External standard curves were constructed from pooled plasma spiked with serial dilutions of G4 or P-G4. Linearity was demonstrated over a dynamic range that spanned four orders of magnitude (0.01-300 μg/mL). Similar to the pharmacokinetics of non-PEGylated GSP mimics of PSGL-1 (88), circulating levels of G4 rapidly declined following administration and were undetectable within 30 minutes. Time-dependent changes in the plasma concentration of G4 were consistent with a terminal half-life of 13.25±5.04 minutes (FIG. 13D). PEGylation significantly extended the half-life of G4 with plasma concentrations well above the limit of quantification at 24 h. Based on the concentration-time profile of P-G4, a terminal half-life of 17.29±8.38 hours was determined using non-compartmental analysis (FIG. 13D).

P-G4 Inhibits Venous Thrombosis without Disruption of Hemostasis

Administration of P-G4 led to a significant decrease in venous thrombus formation after induction of a non-occlusive thrombus by electrolytic injury of the murine inferior vena cava. This effect was equivalent to that observed for enoxaparin (FIG. 13E). Although tail vein transection bleeding time was prolonged after treatment with enoxaparin, bleeding time was unaffected after intravenous administration of P-G4 (FIG. 13F).

TABLE 1
MM/GBSA interaction energies, configurational (−TΔSRTV)
and conformational (−TΔSqc) entropies of PSGL-1
analogues bound to P-selectin at 300°K (kcal/mol).

| | Calculated values of PSGL-1 analogues | | | |
|---|---|---|---|---|
| | 1 | 2 | 3 | 4 |
| ΔG_{MM/GBSA} | −53.9 ± 1.4 | −48.6 ± 3.1 | −49.3 ± 3.8 | −52.7 ± 2.0 |
| −TΔS_{RTV} | 25.6 ± 1.2 | 27.7 ± 1.5 | 28.2 ± 0.0 | 23.9 ± 0.8 |
| −TΔS_{q}^{c} | 2.4 ± 0.5 | 3.3 ± 0.1 | 3.5 ± 0.2 | 5.1 ± 0.4 |
| ΔG_{binding} | −25.9 ± 1.9 | −17.6 ± 3.4 | −17.6 ± 3.8 | −23.7 ± 2.2 |
| | 5 | 6 | 7 | 8 |
| ΔG_{MM/GBSA} | −50.5 ± 5.2 | −48.9 ± 2.2 | −50.9 ± 3.4 | −49.7 ± 2.6 |
| −TΔS_{RTV} | 24.3 ± 0.3 | 24.0 ± 0.4 | 26.8 ± 0.4 | 27.7 ± 0.2 |
| −TΔS_{q}^{c} | 3.0 ± 0.2 | 2.2 ± 0.1 | 2.7 ± 0.1 | 2.1 ± 0.2 |
| ΔG_{binding} | −23.2 ± 5.2 | −22.7 ± 2.2 | −21.4 ± 3.4 | −19.9 ± 2.6 |

SUPPLEMENTARY TABLE 1
Parameters employed for linker molecules in 1 to 8.

| Bond Lengths | kᵣ | r_{eq} | Source | Atom types |
|---|---|---|---|---|
| c3—Os | 285.0 | 1.460 | GLYCAM06 | Cg—Os |
| c3—OS | 285.0 | 1.460 | GLYCAM06 | Cg—Os |

| Bond Angles | k_{θ} | θ_{eq} | Source | Atom types |
|---|---|---|---|---|
| C—CA—C | 63.0 | 120.00 | ff99sb | CA—C—CA |
| CA—C—Os | 70.00 | 120.00 | ff99sb | CA—C—OH |
| CA—C—OS | 70.00 | 120.00 | ff99sb | CA—C—OH |
| c3-c3—Os | 70.00 | 108.50 | GLYCAM06 | Os—Cg—Cg |
| Os-c3-h1 | 60.00 | 110.00 | GLYCAM06 | H1—Cg—Os |
| Cg—Os-c3 | 50.00 | 111.60 | GLYCAM06 | Cg—Os—Cg |
| CT—OS—c3 | 50.00 | 111.60 | GLYCAM06 | Cg—Os—Cg |
| OS—c3-h1 | 60.00 | 110.00 | GLYCAM06 | H1—Cg—Os |
| OS—c3-cc | 50.00 | 109.50 | ff99sb | CM—CT—OS |
| H1—CT—CA | 50.00 | 109.50 | ff99sb | CA—CT—HC |
| Os—CT—CA | 60.00 | 109.50 | ff99sb | C—CT—OS |
| OS—c3-ca | 50.00 | 109.50 | ff99sb | CM—CT—OS |
| OS—c3-c3 | 70.00 | 108.50 | GLYCAM06 | Os—Cg—Cg |
| N—CT—c3 | 80.0 | 109.70 | ff99sb | CT—CT—N |
| CT—c3-hc | 50.0 | 109.50 | ff99sb | CT—CT—HC |
| CT—c3-cd | 63.0 | 113.10 | ff99sb | CT—CT—CC |
| H1—CT—c3 | 50.0 | 109.50 | ff99sb | CT—CT—H1 |
| C—CT—c3 | 63.0 | 111.10 | ff99sb | C—CT—CT |
| cd-c3—Os | 68.51 | 109.01 | GAFF | cd-c3-os |

| Torsion Angles | V₁/2 | V₂/2 | V₃/2 | Source | Atom types |
|---|---|---|---|---|---|
| Cg—Os—C—CA | −0.25 | −3.99 | −0.87 | GLYCAM06 | Cg—Os—Ck—Ck |
| H2—Cg—Os—C | 0.00 | 0.00 | 0.00 | GLYCAM06 | H1—Cg—Os—C |
| Os—Cg—Os—C | 1.08 | 1.38 | 0.96 | GLYCAM06 | Os—Cg—Os—Cg |
| Cg—Os-c3-c3 | 0.00 | 0.00 | 0.16 | GLYCAM06 | Cg—Os—Cg—Cg |
| Cg—Os-c3-h1 | 0.00 | 0.00 | 0.27 | GLYCAM06 | Cg—Os—Cg—H1 |
| H2—Cg—Os-c3 | 0.00 | 0.60 | 0.10 | GLYCAM06 | H2—Cg—Os—Cg |
| Os—Cg—Os-c3 | 1.08 | 1.38 | 0.96 | GLYCAM06 | Os—Cg—Os—Cg |
| Cg—Cg—Os-c3 | 0.00 | 0.00 | 0.16 | GLYCAM06 | Cg—Os—Cg—Cg |
| CT—OS—c3-h1 | 0.00 | 0.00 | 0.27 | GLYCAM06 | Cg—Os—Cg—H1 |
| CT—OS—c3-cc | −0.60 | 0.45 | 0.32 | GLYCAM06 | Cg—Os—Cg—C |
| CT—OS—c3-ca | −0.60 | 0.45 | 0.32 | GLYCAM06 | Cg—Os—Cg—C |
| Cg—Os—CT—CA | −0.60 | 0.45 | 0.32 | GLYCAM06 | Cg—Os—Cg—C |
| CT—OS—c3-c3 | 0.00 | 0.00 | 0.16 | GLYCAM06 | Cg—Os—Cg—Cg |
| N—CT—c3-hc | 0.00 | 0.00 | 0.16 | ff99sb | X—CT—CT—X |
| N—CT—c3-cd | 0.00 | 0.00 | 0.16 | ff99sb | X—CT—CT—X |
| H1—CT—c3-hc | 0.00 | 0.00 | 0.16 | ff99sb | X—CT—CT—X |
| H1—CT—c3-cd | 0.00 | 0.00 | 0.16 | ff99sb | X—CT—CT—X |
| C—CT—c3-hc | 0.00 | 0.00 | 0.16 | ff99sb | X—CT—CT—X |
| C—CT—c3-cd | 0.00 | 0.00 | 0.16 | ff99sb | X—CT—CT—X |
| cd-c3—Os—Cg | 0.00 | 0.10 | 0.383 | GAFF | c3-c3-os-c3 |

SUPPLEMENTARY TABLE 2
Key intermolecular hydrogen bond distances and occupancies
between P-selectin and different ligands.

| Ligand | | P-selectin | | | |
|---|---|---|---|---|---|
| residue | Atom | Atom | x-ray | GSnP-6 | G4 |
| Neu5Ac | O1^{a} | Y48—Oη | 2.5^{b} | 2.6 ± 0.1 (100)^{c,d} | 2.8 ± 0.2 (100)^{c,d} |
| | O4 | S99—Oγ | 3.1 | 3.3 ± 0.1 (53) | 2.9 ± 0.2 (74) |
| Core-2 Gal | O4 | Y94—Oη | 2.7 | 2.7 ± 0.1 (100) | 2.9 ± 0.2 (83) |
| | O6 | E92—Oε^{a} | 2.5 | 2.8 ± 0.2 (100)^{c,d} | 2.9 ± 0.2 (100)^{c,d} |
| Fuc | O2 | E88—Oε^{a} | 2.6^{b} | 2.9 ± 0.1 (67) | 2.7 ± 0.1 (100)^{c,d} |
| | O3 | N105—Nδ2 | 2.9 | 2.9 ± 0.2 (75) | 3.4 ± 0.1 (1) |
| | O3 | E107—Oε^{a} | 2.7 | 2.9 ± 0.2 (100)^{c,d} | 2.8 ± 0.2 (98) |
| | O4 | E80—Oε^{a} | 2.6 | 2.8 ± 0.3 (100)^{c,d} | 2.9 ± 0.2 (100)^{c,d} |
| | O4 | N82—Nδ2 | 3.0 | 2.8 ± 0.2 (88) | 2.8 ± 0.1 (95) |
| SO₃⁻ 605 | O^{e} | K8—Nζ | — | 3.0 ± 0.2 (31) | 3.0 ± 0.2 (29) |
| | O^{e} | K112—Nζ | — | 2.9 ± 0.2 (12) | 2.9 ± 0.2 (6) |
| SO₃⁻ 607 | O^{e} | S46—Oγ | 3.3 | 2.8 ± 0.2 (48) | 2.8 ± 0.2 (42) |
| | O^{e} | S47—Oγ | 3.0 | 2.9 ± 0.3 (100)^{c,d} | 2.9 ± 0.2 (100)^{c,d} |
| | O^{e} | H114—Nε2 | 2.7 | 2.8 ± 0.1 (100)^{c,d} | 2.8 ± 0.1 (100)^{c,d} |
| SO₃⁻ 610 | O^{e} | R85—Nη1 | 2.7 | 2.9 ± 0.2 (8) | 3.1 ± 0.2 (4) |
| | O^{e} | R85—Nη2 | 3.7 | 3.0 ± 0.2 (15) | 2.9 ± 0.2 (6) |

^{a}Hydrogen bonds with both oxygen atoms in carboxylate group were counted.
^{b}In Å.
^{c}Percentage (%) based on a distance between non-hydrogen atoms of less than 3.5 Å. When multiple hydrogen bonds are formed between two heavy atoms through different hydrogens, the occupancy of the interaction listed is the sum of all the individual hydrogen bonds and the distance is the average of all the individual hydrogen bonds.
^{d}The occupancy of the interactions between two heavy atoms, calculated as the sum of all the individual hydrogen bonds through different hydrogens, is greater than 100%.
^{e}Oxygen atom in SO₃⁻ group of tyrosine sulfate or tyrosine sulfonate.

SUPPLEMENTARY TABLE 3
Per-residue MM/GBSA interaction energiesa and entropiesb
for different ligands binding with P-selectin.

| | |
|---|---|
| GSnP-6 | 1 |

Tyrosine sulfonate

| | | |
|---|---|---|
| YC605^{c} | −1.5 ± 0.1 | −1.5 ± 0.6 |
| YC607^{c} | −5.9 ± 0.0 | −6.1 ± 0.1 |
| YC610^{c} | −0.4 ± 0.2 | −0.5 ± 0.1 |
| SO3- 605^{d} | −2.6 ± 0.2 | −2.3 ± 0.7 |
| SO3- 607^{d} | −3.7 ± 0.1 | −3.7 ± 0.1 |
| SO3- 610^{d} | −1.0 ± 0.3 | −0.8 ± 0.2 |
| Subtotal | −15.1 ± 0.4 | −14.9 ± 1.0 |

Glycan

| | | |
|---|---|---|
| Neu5Ac | −2.9 ± 0.0 | −2.8± 0.1 |
| Core-2 Gal | −4.2 ± 0.1 | −4.1 ± 0.2 |
| GlcNAc | −3.5 ± 0.2 | −3.6 ± 0.0 |
| Fuc | −7.6 ± 0.1 | −7.6 ± 0.0 |
| GalNAc/click/aryl | −0.5 ± 0.1 | −1.5 ± 0.1 |
| Gal | 0.2 ± 0.0 | |
| Subtotal | −18.5 ± 0.2 | −19.6 ± 0.2 |

Amino acids

| | | |
|---|---|---|
| K603^{e} | 1.6 ± 0.1 | 1.7 ± 0.1 |
| E604 | −0.7 ± 0.1 | −0.9 ± 0.1 |
| E606 | −1.3 ± 0.1 | −1.3 ± 0.1 |
| L608 | −2.5 ± 0.5 | −2.2 ± 0.2 |
| D609 | −1.4 ± 0.3 | −1.8 ± 0.1 |
| D611 | −0.9 ± 0.0 | −0.9 ± 0.1 |
| F612 | −2.3 ± 0.9 | −2.2 ± 0.1 |
| L613 | −4.1 ± 0.6 | −4.2 ± 0.2 |
| P614 | −1.2 ± 0.2 | −2.7 ± 0.9 |
| E615 | −1.2 ± 0.1 | −1.2 ± 0.0 |
| T616^{f} | −1.4 ± 0.1 | −2.2 ± 0.2 |
| E617 | −0.9 ± 0.1 | −0.8 ± 0.1 |
| P618 | −0.6 ± 0.1 | −0.7 ± 0.0 |
| Subtotal | −16.9 ± 1.3 | −19.4 ± 1.0 |
| Total MM/GBSA Energy | −50.5 ± 1.4 | −53.9 ± 1.4 |

Entropy

| | | |
|---|---|---|
| −TΔS_{RTV} | 23.8 ± 0.6 | 25.6 ± 1.2 |
| −TΔS_{q}^{c} | 3.1 ± 0.2 | 2.4 ± 0.5 |
| Binding Free energy | −23.6 ± 1.4 | −25.9 ± 1.8 |

^{a}In kcal/mol.
^{b}At 300K.
^{c}Tyrosine sulfonate (YC) not including SO₃⁻ group.
^{d}SO₃⁻ is considered as a residue in energy calculation.
^{e}Residue numbering is based on the crystal structure (PDB ID: 1G1S).
^{f}Glycosylation site.

### Discussion

There is a need to develop P-selectin inhibitors that exhibit both high affinity and specificity. First generation synthetic small molecule P-selectin antagonists were designed to mimic the tetrasaccharide sLe^{x} moiety of PSGL-1, but failed to account for crucial contributions of multiple clustered tyrosine sulfates (89-94). This historical failure is crucial, because high-affinity binding of P-selectin to PSGL-1 requires stereospecific interactions with both clustered tyrosine sulfates and the sLe^{x}-containing hexasaccharide epitope (95-97). Furthermore, earlier attempts to synthesize glycosulfopeptide (GSP) mimics of the N-terminus of PSGL-1 were limited by poor selectivity (98), incompatible protecting groups (99), and the instability of tyrosine sulfates (100, 101). As detailed elsewhere (44, 65, 78, 102, 103), the design and chemoenzymatic synthesis of a broad range of GSP mimics of PSGL-1 was previously reported, and it was determined that hydrolytically labile tyrosine sulfates could be replaced with bioisosteric sulfonate analogues. Library screening identified GSnP-6, which is exceptionally stable and binds human P-selectin with low nanomolar affinity, preventing venous thrombus formation via inhibition of PSGL-1/P-selectin mediated platelet-leukocyte aggregation without affecting the coagulation pathway and, thereby, maintaining normal hemostasis.

In an analysis of the binding interactions of PSGL-1 analogues with P-selection, a computational model was first validated by reproducing all structural attributes of the PSGL-1/P-selectin interaction as defined by crystallographic data and experimental investigations. In particular, all observed H-bonds and salt bridges were detected and the interactions between tyrosine sulfate and P-selectin surface residues were most stable when histidine H114 was fully-protonated. Consistent with experimental findings, computed interaction energies were able to correctly rank the relative contributions of unique PSGL-1 structural features towards P-selectin binding (44). Specifically, the contribution of fucose was determined to be equivalent to that of the total sulfate residues, and greater than Neu5Ac. Moreover, simulations identified that the second sulfate made a larger contribution than the other two. The predicted absolute interaction energies of P-selectin with either PSGL-1 or GSnP-6 were statistically equivalent in agreement with experimental data comparing GSnP-6 with the native ligand (44). In the current investigation, synthetic considerations along with in silico results identified compound 1 (G4) as a lead candidate, which MD simulations had predicted would retain binding energy to P-selectin comparable to PSGL-1 and GSnP-6. Consistent with these predictions, G4 displayed low nanomolar binding affinity to P-selection and both this compound and its PEGylated derivative, P-G4, effectively inhibited P-selectin binding to PSGL-1 in a dose-dependent manner in cell-based assays and reduced both human and mouse neutrophil-platelet and monocyte-platelet aggregation. Significantly, P-G4 inhibited venous thrombosis in a preclinical model of VTE without disruption of hemostasis. Importantly, the development of a total chemical synthesis for P-G4 enables the scalable production of this drug candidate.

Inhibitors of the PSGL-1/P-selectin pathway, including recombinant PSGL-1 (rPSGL-Ig) (104-106), monoclonal antibodies (mAbs) (107-109), small molecule antagonists (110-113), and oligonucleotide aptamers (114) have been shown to inhibit venous thrombus formation, accelerate clot resolution, and decrease local inflammatory responses, as well as late vein wall fibrosis in rodent, feline, and primate models. These results are consistent with studies in transgenic mouse models, which have all demonstrated that P-selectin mediates leukocyte recruitment, tissue factor release, and fibrin deposition, thereby, playing a critical role in the initiation and propagation of venous thrombosis (43, 115-117).

Although supporting the significance of the P-selectin/PSGL-1 pathway in VTE, approaches for inhibiting this pathway have faced a number of hurdles to clinical translation. In particular, the production of recombinant glycoproteins, such as rPSGL-Ig, is often associated with imprecise post-translation modifications, especially under sensitive culture conditions in non-human cell lines with a failure to preserve glycan fine structure (118). Moreover, protein modifications can alter pharmacokinetics, and induce immunogenicity and hypersensitivity (119, 120). In part, initial studies with rPSGL-Ig were only feasible after transfection of Chinese hamster ovarian cell lines with core-2 β1,6-GlcNAc transferase (121-123). The production of therapeutic glycoproteins bearing complex glycans requires exquisite control of an inherently sensitive complex biologic process, which may be difficult to achieve on a commercial scale (124).

Several antibodies have been developed targeting the PSGL-1/P-selectin pathway. Crizanilizumab (SelG1), an anti-P-selectin mAb, was recently approved for the treatment of sickle cell disease (125) and SelK2, an anti-PSGL-1 mAb, is in ongoing clinical trials. However, all therapeutic antibodies are susceptible to an immune response, which may lead to loss of drug efficacy, especially when repeated dosing and long-term therapy are required (126). The recognition of anti-drug antibody production to both chimeric and humanized mAbs emphasizes the persistent challenges that exist towards reducing antibody immunogenicity (127).

The development of synthetic P-selectin antagonists continues to be pursued, but the identification of inhibitors with high affinity and specificity remains a significant challenge (128). Synthetic small molecule inhibitors largely display micromolar affinity for P-selectin (GMI-1070 IC₅₀ 423 μM; PSI-697 IC₅₀150 μM; PSI-421 IC₅₀ 225 μM) (129, 130) as compared to nanomolar affinity observed for native P-selectin/PSGL-1 interactions (K_{d} 70-300 nM) (131, 132). While PSI-697 and PSI-421 have been evaluated in rodent (110, 111) and primate (112, 113) models of VTE with enhanced thrombus resolution, disappointing clinical outcomes for these compounds have been attributed, at least in part, to their limited binding affinity. For example, PSI-697 failed to inhibit platelet-monocyte aggregation in a phase I clinical trial despite in vitro efficacy and GMI-1070 has required gram-scale dosing in clinical studies of sickle cell disease (133, 134). The potential for P-selectin inhibition with oligonucleotide aptamers has been demonstrated in a baboon model of VTE, but further studies are lacking (114). Furthermore, significant translational challenges exist for aptamer therapy, including enzymatic degradation, endosomal sequestration, and rapid renal clearance, along with a limited understanding of the safety profile of this class of compounds (135). In summary, the variable effectiveness of many of the previously reported P-selectin antagonists as a consequence of limited affinity and specificity, combined with the inherent challenges associated with recombinant glycoproteins and therapeutic antibodies have hindered the introduction of P-selectin targeted therapy for VTE.

As a high affinity P-selectin inhibitor, P-G4 presents a number of advantages as a drug candidate. The sLe^{x} moiety in P-G4 is identical to that in PSGL-1 and the peptide sequence similar to the PSGL-1 epitope responsible for binding to P-selectin, with modification of the tyrosine sulfates to sulfonates. Therefore, P-G4 is unlikely to induce an immunogenic response. As noted, the formation of anti-drug or neutralizing antibodies to a biologic, such as a therapeutic antibody or protein, is a major cause of loss of drug efficacy and a significant burden in drug development. Further, unlike a biologic, whose systemic clearance may require several weeks to complete (crizanlizumab t_{1/2} 10.6 days; rPSGL-Ig t_{1/2} 4 days) (123, 125), the short half-life of P-G4 (t_{1/2} 17 h) is advantageous, particularly, in cancer patients. Finally, P-G4 is a synthetic compound and can be stored as a lyophilized powder and from the perspective of supplies and logistics management, particularly during the development phase, more cost effective and feasible than a therapeutic antibody supplied in solution. Of note, there are approximately 80 peptide drugs on the global market, with more than 150 peptides in clinical development (136, 137), along with multiple FDA-approved PEGylated proteins and peptides in clinical trials (138, 139). Indeed, extracellular receptors are ideal candidates for peptide drugs, especially those targets that require a large surface area for a therapeutic response.

In studies, in vivo administration of P-G4 resulted in a 60% reduction in thrombus weight. Notably, this effect is equivalent to that obtained by treatment with low molecular weight heparin, yet is not associated with an increase in bleeding time. The electrolytically induced vena cava injury model employed in these studies creates a consistent, partially occlusive thrombus with continuous exposure to venous flow and reliable drug exposure levels. As such, this model is the preferred small animal model for comparing anti-thrombotic agents (140).

VTE occurs disproportionately in a number of specific types of cancers, which are among those most frequently reported, including lung, breast, colorectal, and pancreatic cancer (13, 14, 141). The rate of VTE in hospitalized cancer patients in the US increased from 3.4% in 1995 to 6.5% in 2012, consistent with an unequivocal increase in incidence. Low molecular weight heparin, such as dalteparin, has an established history of clinical effectiveness in the treatment of VTE and is first-line therapy for cancer associated VTE (142). However, dalteparin, which is the only FDA approved anticoagulant for prevention of recurrent VTE in cancer patients carries an FDA black box warning for risk of epidural or spinal hematoma and has also been associated with a risk of thrombocytopenia that may lead to either terminating or reducing the dose in a significant proportion of patients. Thus, the equivalent anti-thrombotic and anti-inflammatory effects of P-G4 without disruption of hemostasis is a significant finding given the morbidity associated with anticoagulants even at reduced dosing regimens. It is noteworthy that plasma P-selectin levels represents a validated biomarker of patients at increased risk of cancer-associated VTE (39, 40). As such, plasma P-selectin may serve as an important biomarker of the relative effectiveness of P-G6 inhibition in at-risk individuals.

In summary, blockade of the P-selectin/PSGL-1 pathway by P-G4 inhibited murine and human leukocyte/P-selectin binding in a dose dependent manner and reduced platelet-leukocyte aggregation. P-G4 inhibited venous thrombosis in a pre-clinical model of VTE without impairing hemostasis. These findings support the development of P-G4 for the prevention of VTE, particularly among cancer patients who are at increased risk of bleeding from direct oral anticoagulants and low molecular weight heparin.

### Example 2: Synthesis of Arl GSnP-4

### N-(tert-butoxycarbonyl)-O-(3-benzyloxy)-phenyl-L-threonine tert-butyl ester (22)

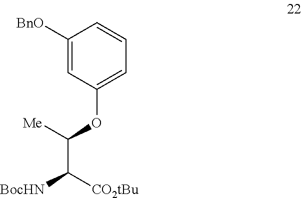 [SMILES: C[C@@H](Oc1cccc(OCc2ccccc2)c1)[C@H](NC(=O)OC(C)(C)C)C(=O)OC(C)(C)C]

N-Boc protected threonine t-butyl ester (200 mg, 0.727 mmol, 1 equiv), 3-(benzyloxy)phenyl boronic acid (414 mg, 1.81 mmol, 2.5 equiv), and activated 4 Å molecular sieves (250 mg) in a 25 mL round bottom flask were diluted in acetonitrile (2.6 mL, 0.275 M). Copper (II) acetate (198 mg, 1.09 mmol, 1.5 equiv) was then added followed by pyridine (583 μL, 2.91 mmol, 4 equiv) dropwise. The crude reaction was capped with a rubber septum under Ar and stirred for 18 hours. The reaction was then diluted in ethyl acetate (300 mL), filtered through celite (5 g), washed with water (2×200 mL), brine (2×200 mL), and dried with anhydrous Na₂SO₄. Ethyl acetate was removed in vacuo and the crude mixture was purified by flash column chromatography to afford 22 as a viscous oil in 68% yield. R_{f}=0.54 (n-hexanes:ethyl acetate=5/1). ¹H NMR (600 MHz, CDCl₃): δ 7.45-7.32 (m, 5H, PhH), 7.18 (t, J=7.6 Hz, 1H, PhH), 6.61 (dd, J=8.7, 2.2 Hz, 1H), 6.56 (s, 1H), 6.54-6.51 (m, 1H), 5.37 (d, J=9.7 Hz, 1H), 5.07-5.02 (m, 2H), 4.96-4.90 (m, 1H), 4.39 (dd, J=9.5, 2.2 Hz, 1H), 1.51 (s, 9H), 1.36 (s, 9H), 1.35-1.32 (m, 3H). ¹³C NMR (150 MHz, CDCl₃): δ 169.4, 160.0, 158.6, 156.1, 136.9, 129.9, 128.6, 127.9, 127.5, 108.2, 107.7, 103.1, 82.22, 79.86, 74.42, 70.01, 58.30, 28.35, 27.91, 16.11. HRMS: (ESI-MS m/z) calc. mass for [C₂₈H₃₅NO₆+Na⁺] 480.2357, found 480.2356.

### N-(tert-butoxycarbonyl)-O-(3-phenoxy)-L-threonine tert-butyl ester (23)

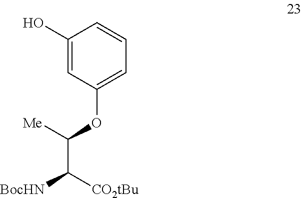 [SMILES: C[C@@H](Oc1cccc(O)c1)[C@H](NC(=O)OC(C)(C)C)C(=O)OC(C)(C)C]

Compound 22 (120 mg) was then diluted in methanol (10 mL) and palladium was added (150 mg) under hydrogen gas at 1 atm. The reaction was completed after 1 hour at rt and the crude mixture was filtered through celite (2 g) and solvents were removed in vacuo. Purification via flash column chromatography gave 23 as a white foam in 99% yield. R_{f}=0.52 (n-hexanes:ethyl acetate=2/1). ¹H NMR (600 MHz, CDCl₃): δ 7.09 (t, J=8.65 Hz, 1H, PhH), 6.49-6.39 (m, 3H, PhH), 5.44 (d, J=9.59 Hz, 1H), 4.94-4.88 (m, 1H), 4.39 (d, J=9.59 Hz, 1H), 1.49 (s, 3H), 1.35 (s, 1H), 1.34 (d, J=6.40 Hz, 1H, CH₃)¹³C NMR (150 MHz, CDCl₃): δ 169.5, 158.6, 158.4, 157.4, 156.4, 130.1, 108.6, 107.8, 103.3, 82.53, 80.25, 74.20, 58.28, 16.15. HRMS: (ESI-MS m/z) calc. mass for [C₁₉H₂₉NO₆+Na⁺] 390.1887, found 390.1886.

### N-[((9H-fluoren-9-yl)methoxy)carbonyl]-O-(3-phenoxy)-L-threonine tert-butyl ester (24)

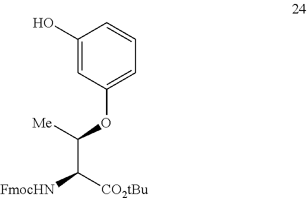

Compound 23 (100 mg, 0.272 mmol, 1 equiv) was dissolved in 4 M HCl in 1,4-dioxane (10 mL) and let stir vigorously at 0° C. and slowly warmed to rt. Once the reaction was complete, the reaction mixture was removed in vacuo. The crude mixture was then dissolved in 1:1 (v/v) mixture of 1,4-dioxane/water (10 mL) at 0° C. and sodium bicarbonate (57 mg, 0.68 mmol, 2.5 equiv) was added. Subsequently, Fmoc-OSu (119 mg, 0.354 mmol, 1.3 equiv) was added slowly and the reaction mixture was warmed to rt and stirred for 18 hours. The reaction mixture was diluted in dichloromethane (200 mL), washed with water (2×200 mL), brine (2×200 mL), then dried with anhydrous Na₂SO₄. The solvents were removed in vacuo then the crude mixture was subjected to flash chromatography, which rendered purified 24 as a white foam in 78% yield. R_{f}=0.45 (n-hexanes:ethyl acetate=2/1). ¹H NMR (600 MHz, CDCl₃): δ 7.80 (dd, J=7.5, 2.6, 2H), 7.67 (t, J=6.8 Hz, 2H), 7.45-7.41 (m, 2H), 7.37-7.33 (m, 2H), 7.14 (t, J=8.2 Hz 1H), 6.54-6.51 (m, 1H), 6.48-6.45 (m, 1H), 5.66 (d, J=9.6 Hz, 1H), 5.05 (br s., 1H), 4.97-4.91 (m, 1H), 4.49 (dd, J=9.6, 2.5 Hz, 1H), 4.46 (d, J=7.4 Hz, 2H), 4.29 (t, J=6.8 Hz, 1H), 1.39 (s, 9H), 1.35 (d, J=6.3 Hz, 3H), 1.29-1.27 (m, 3H). ¹³C NMR (150 MHz, CDCl₃): δ 169.0, 158.6, 156.8, 156.7, 143.9, 143.7, 141.3, 130.2, 127.7, 127.1, 125.2, 120.0, 108.5, 108.1, 103.3, 82.6, 74.2, 62.3, 58.5, 47.2, 29.7, 27.9, 16.1. HRMS: (ESI-MS m/z) calc. mass for [C₂₉H₃₁NO₆+Na⁺] 512.2044, found 512.2044.

### N-[((9H-fluoren-9-yl)methoxy)carbonyl]-O-3-(phenoxy)-[3,4,6-tri-O-acetyl-2-deoxy-(2,2,2-trichloroethoxycarbonylamino)-β-D-glucopyranosyl]-L-threonine tert-butyl ester (25)

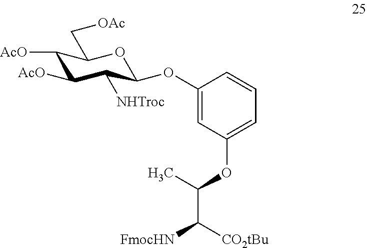

Glycosyl imidate (200 mg, 0.321 mmol, 1 equiv) and 24 (204 mg, 0.417 mmol, 1.3 equiv) were dissolved in dichloromethane with activated 4 Å molecular sieves for 1 hour at rt. The reaction mixture was cooled to −30° C. and TMSOTf (11 μL, 0.064 mmol, 0.2 equiv) was slowly added and let stir for 1 hour. The reaction was quenched with triethylamine (0.2 mL) and the reaction mixture diluted in dichloromethane was filtered through celite (5 g), washed with sodium bicarbonate (200 mL), brine (200 mL) and dried with anhydrous Na₂SO₄. Solvents were removed in vacuo and the crude mixture was subjected to flash chromatography, which gave the purified 25 as a white foam in 91% yield. R_{f}=0.17 (n-hexanes:ethyl acetate=2/1). ¹H NMR (600 MHz, CDCl₃): δ 7.81 (d, J=7.13 Hz, 2H), 7.66 (t, J=7.13 Hz, 2H), 7.43 (app. t., J=7.34, 1H), 7.34 (q, J=6.8 Hz, 2H), 7.18 (t, J=8.08 Hz, 1H), 6.78 (s, 1H), 6.68-6.63 (m, 2H), 5.80 (d, J=9.13 Hz, 1H), 5.67 (d, J=8.48 Hz, 1H), 5.35 (t, J=10.1 Hz, 1H), 5.18-5.10 (m, 2H), 4.87-4.80 (m, 1H), 4.76-4.65 (m, 2H) 4.55-4.47 (m, 3H), 4.35-4.25 (m, 2H), 4.19-4.11 (m, 2H), 3.97 (q, J=9.28 Hz, 1H), 3.84-3.77 (m, 1H), 2.1 (s, 3H), 2.04 (s, 6H), 1.43 (s, 9H), 1.33 (d, J=6.05 Hz, 3H). ¹³C NMR (150 MHz, CDCl₃): δ 170.6, 170.5, 169.4, 168.8, 158.5, 158.2, 156.6, 154.3, 143.8, 141.36, 141.34, 130.1, 127.8, 127.1, 125.2, 125.1, 120.09, 120.04, 111.0, 110.8, 105.2, 100.2, 95.5, 82.9, 74.4, 73.5, 72.0, 71.8, 68.5, 67.4, 62.1, 57.3, 56.1, 47.1, 27.9, 20.7, 20.6, 15.8, 14.2. HRMS: (ESI-MS m/z) calc. mass for [C₄₄H₄₉Cl₃N₂O₁₅+Na⁺] 973.2091, found 973.2091.

### N-[((9H-fluoren-9-yl)methoxy)carbonyl]-O-(3-phenoxy) 3,4,6-tri-O-acetyl-2-deoxy-(2-acetamido)-β-D-glucopyranosyl-L-threonine tert-butyl ester (26)

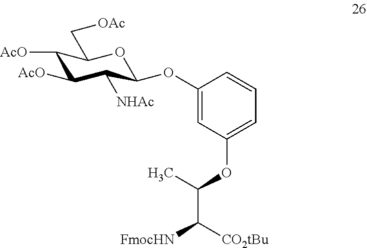

Activated zinc (421 mg), acetic anhydride (5 mL), and acetic acid (0.5 mL) were added to compound 25 (100 mg, 0.105 mmol, 1 equiv), which was dissolved in THF (5 mL) and let stir vigorously for 2 hours at rt under Ar. Once the reaction was complete, the reaction mixture diluted in ethyl acetate (300 mL) was filtered through Celite (4 g), washed with sodium bicarbonate (2×150 mL), brine (200 mL), and dried with anhydrous Na₂SO₄. Solvents were removed in vacuo and the crude mixture was subjected to flash chromatography, which gave the purified 26 as a white foam in 78% yield. R_{f}=0.16 (n-hexanes:ethyl acetate=1/1.5). ¹H NMR (600 MHz, CDCl₃): δ 7.78 (d, J=7.36 Hz, 2H), 7.64 (t, J=7.93 Hz, 2H), 7.43-7.38 (m, 2H), 7.32 (q, J=6.91 Hz, 2H), 7.16 (t, J=8.06 Hz, 1H), 6.66 (s, 1H), 6.63-6.59 (m, 2H), 5.76 (d, J=9.21 Hz, 1H), 5.62 (d, J=8.63 Hz, 1H), 5.34-5.28 (m, 1H), 5.17-5.01 (m, 2H), 4.88-4.80 (m, 1H), 4.50-4.42 (m, 3H), 4.29-4.24 (m, 2H), 4.19-4.08 (m, 3H), 3.84-3.79 (m, 1H), 2.06 (s, 3H), 2.03 (s, 3H), 2.02 (s, 3H), 1.92 (s, H), 1.38 (s, 9H), 1.31 (d, J=6.33 Hz, 1H). ¹³C NMR (150 MHz, CDCl₃): δ 170.8, 170.6, 170.2, 169.4, 168.9, 158.4, 158.3, 156.6, 143.8, 143.7, 141.3, 130.1, 127.8, 127.7, 127.1, 125.2, 125.1, 120.08, 120.04, 110.6, 110.1, 105.0, 99.4, 82.8, 73.7, 72.2, 72.0, 68.4, 67.3, 62.1, 57.9, 54.5, 47.2, 27.9, 23.3, 20.7, 20.67, 20.62, 15.9. HRMS: (ESI-MS m/z) calc. mass for [C₄₃H₅₀N₂O₁₄+Na⁺] 841.3154, found 841.3154.

### N-[(9H-fluoren-9-yl)methoxy)carbonyl]-O-(3-phenoxy)-3,4,6-tri-O-acetyl-2-deoxy-(2-acetamido)-β-D-glucopyranosyl-L-threonine (27)

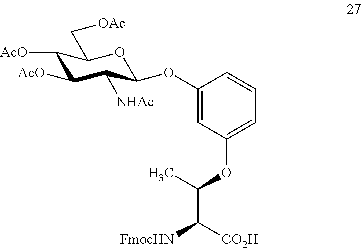

At 0° C., compound 26 (55 mg, 0.058 mmol, 1 equiv) was dissolved in dichloromethane (10 mL). Triethylsilane (1.8 μL, 0.012 mmol, 0.2 equiv) and trifluoroacetic acid (5 mL) were added dropwise, respectively. The reaction mixture was slowly warmed to rt, stirred for 3 h, and concentrated in vacuo to afford a yellow oil, which was diluted in ethyl acetate and washed carefully with sodium bicarbonate then 1 N HCl to acidify the crude mixture to a pH at ˜1-1.5, from which the crude mixture was extracted in ethyl acetate. The organic layer was washed with brine then dried with anhydrous Na₂SO₄. The solvents were dried in vacuo then the resulting mixture was subjected to flash chromatography, which gave the purified 27 as a yellow oil in 84% yield. R_{f}=0.08 (ethyl acetate:methanol=5/1). ¹H NMR (600 MHz, CD₃OH): δ 7.828 (d, J=7.5 Hz, 2H), 7.75-7.67 (m, 2H), 7.45-7.30 (m, 4H), 7.19 (t, J=8.6 Hz, 1H), 6.70 (dd, J=8.2, 1.5 Hz, 1H), 6.67 (s, 1H), 6.63 (dd, J=8.1, 1.7 Hz, 1H), 5.02-4.98 (m, 1H), 4.50-4.45 (m, 1H), 4.44-4.39 (m, 1H), 4.34-4.27 (m, 2H), 4.15 (dd, J=12.2, 1.9 Hz, 1H), 4.01 (app. t., J=9.14 Hz, 1H), 4.01-3.96 (m, 1H), 2.05 (s, 3H), 2.031 (s, 3H), 2.027 (s, 3H), 1.95 (s, 3H), 1.31 (d, J=6.3 Hz, 1H). ¹³C NMR (150 MHz, CD₃OH): δ 172.3, 170.9, 170.4, 169.9, 158.6, 158.3, 157.6, 143.9, 143.8, 141.2, 129.6, 127.4, 126.8, 124.9, 124.8, 119.6, 119.5, 110.1, 105.2, 98.4, 74.2, 72.4, 71.6, 68.7, 66.7, 61.9, 54.2, 47.06, 21.4, 19.3, 19.2, 19.1, 15.2. HRMS: (ESI-MS m/z) calc. mass for [C₃₉H₄₂N₂O₁₄+Na⁺] 785.2528, found 785.2528.

### 4-O-{2-O-acetyl-4,6-benzylidene-3-O-[methyl-(5-acetamido-7,8,9-tri-O-acetyl-5-N,4-O-carbonyl-3,5-dideoxy-D-glycero-α-D-galacto-non-2-ulopyranosyl)onate]-β-D-galactopyranosyl}-3-O-(2,3,4-tri-O-benzyl-α-L-fucopyranosyl)-6-O-benzyl-2-deoxy-2-(2,2,2-trichlorethoxy-carbonylamino)-α,β-D-glucopyranose (29)

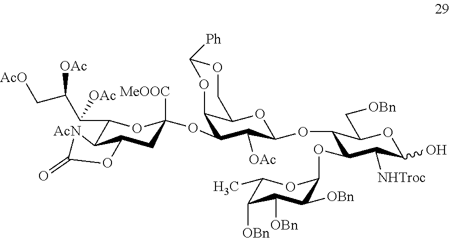

In a plastic round bottom flask, compound 28 (200 mg, 0.108 mmol, 1 equiv) was dissolved in pyridine (4 mL) and Olah's reagent (0.4 mL) was added dropwise at 0° C. After 17 hours at rt, the reaction was diluted in CH₂Cl₂ (10 mL) and water (10 mL). Sodium bicarbonate (200 mg) was slowly added to quench the reaction. The organic layer was separated, collected, and washed with saturated sodium bicarbonate solution (20 mL) twice. The aqueous layer was discarded and the organic layer was washed with brine (20 mL) and dried over Na₂SO₄. The solvents were removed in vacuo and the crude material was subjected to flash column chromatography, which gave 29 in 92% yield. R_{f}=0.42 (n-hexanes:ethyl acetate=1/2). ¹H NMR (600 MHz, CDCl₃): δ 7.61-7.53 (m, 2H), 7.39-7.17 (m, 30H), 5.67 (dd, J=8.99, 1.65 Hz, 1H), 5.66-5.62 (m, 1H), 5.57-5.53 (m, 1H), 5.45 (s, 1H), 5.26 (app. t., J=9.49 Hz, 1H), 5.13-5.09 (m, 1H), 5.02 (br. s., 1H), 4.80-4.74 (m, 2H), 4.73-4.66 (m, 4H), 4.64-4.54 (m, 5H), 4.49 (dd, J=9.62, 1.65 Hz, 1H), 4.45 (dd, J=10.13, 3.92 Hz, 1H), 4.39-4.35 (m, 2H), 4.34-4.29 (m, 1H), 4.15-3.94 (m, 8H), 3.89-3.83 (m, 7H), 3.71 (app. t., J=10.13 Hz), 1H), 3.56 (s, 3H), 3.27 (m, 1H), 3.09 (dd, J=12.05, 3.34 Hz, 1H), 2.51 (s, 3H), 2.16 (s, 3H), 2.14 (s, 3H), 2.07 (s, 3H) 1.99 (app. t., J=12.05 Hz, 1H), 1.97 (s, 3H), 1.14 (d, J=7.74 Hz, 3H). ¹³C NMR (150 MHz, CDCl₃): δ 171.9, 171.2, 170.9, 170.4, 170.26, 169.7, 169.4, 169.0, 168.4, 153.5, 139.7, 138.7, 138.1, 137.9, 128.5, 128.3, 128.2, 127.9, 127.6, 127.3, 127.1, 126.9, 126.0, 125.7, 100.0, 99.43, 97.59, 97.28, 95.23, 91.66, 79.25, 78.57, 75.86, 75.16, 75.09, 74.89, 74.67, 74.41, 73.22, 73.10, 72.67, 71.80, 71.71, 71.07, 69.69, 68.99, 68.19, 68.09, 66.38, 66.25, 63.31, 60.44, 59.13, 58.93, 56.97, 53.23, 52.96, 37.21, 24.69, 24.27, 21.32, 21.09, 20.96, 20.88, 20.74, 20.69, 20.59, 16.17, 16.11, 14.21.

### 4-O-{2-O-acetyl-4,6-benzylidene-3-O-[methyl-(5-acetamido-7,8,9-tri-O-acetyl-5-N,4-O-carbonyl-3,5-dideoxy-D-glycero-α-D-galacto-non-2-ulopyranosyl)onate]-β-D-galactopyranosyl}-3-O-(2,3,4-tri-O-benzyl-α-L-fucopyranosyl)-6-O-benzyl-2-deoxy-2-(2,2,2-trichlorethoxy-carbonylamino)-α,β-D-glucopyranosyl trichloroacetimidate (30)

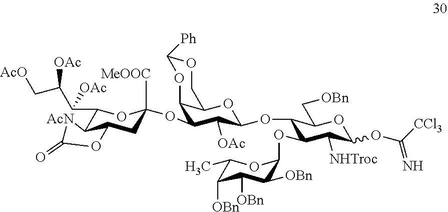

At 0° C., compound 29 (160 mg, 0.099 mmol, 1 equiv) was dissolved in dichloromethane (10 mL). Trichloroacetonitrile (99 μL, 10 equiv) was added and 1,8-diazabicyclo(5.4.0)undec-7-ene (3 μL, 0.2 equiv) was added dropwise. The reaction was halted after 1 hour and the solvents were removed in vacuo before it was subjected to flash column chromatography to render 30 as a white foam in 64% yield. R_{f}=0.48 (n-hexanes:ethyl acetate=1/1). ¹H NMR (600 MHz, CDCl₃): δ 8.67 (s, 1H), 7.59 (d, J=7.47 Hz, 1H), 7.39-7.19 (m, 25H), 7.14 (d, J=7.03 Hz, 1H), 6.38 (d, J=3.96 Hz, 1H), 5.66 (dd, J=9.03, 1.40 Hz, 1H), 5.55 (ddd, J=9.19, 7.32, 2.80 Hz, 1H), 5.46 (s, 1H), 5.33-5.25 (m, 3H), 4.86 (d, J=8.90 Hz, 1H), 4.82-4.60 (m, 8H), 4.49 (dd, J=8.49, 1.21 Hz, 1H), 4.45 (dd, J=10.11, 4.05 Hz, 1H), 4.38-4.31 (m, 3H), 4.28-4.06 (m, 5H), 4.05-3.77 (m, 10H), 3.69 (app. t., J=10.52 Hz, 1H), 3.55 (s, 3H), 3.17 (br. s., 1H), 3.09 (dd, J=12.54, 3.24 Hz, 1H), 2.51 (s, 3H), 2.16 (s, 3H), 2.09 (s, 3H), 2.05 (s, 3H), 1.97 (s, 3H), 1.98 (app. t., J=12.54 Hz, 1H), 1.19 (d, J=7.26 Hz, 3H). ¹³C NMR (150 MHz, CDCl₃): δ 172.0, 170.7, 170.3, 169.6, 168.4, 154.1, 153.4, 139.7, 139.6, 138.1, 137.9, 128.9, 128.4, 128.2, 128.1, 127.9, 127.8, 127.6, 127.5, 126.9, 126.8, 125.9, 99.92, 99.47, 98.04, 97.28, 95.30, 95.28, 78.87, 78.61, 75.17, 75.03, 74.64, 73.75, 73.23, 73.11, 72.72, 71.80, 71.65, 69.63, 68.98, 68.00, 66.72, 66.29, 63.27, 59.11, 56.19, 53.22, 37.19, 29.71, 24.68, 21.34, 20.94, 20.87, 20.72, 16.05.

### N-(tert-butoxycarbonyl)-O-3-(phenoxy)-[4-O-{2-O-acetyl-4,6-benzylidene-3-O-[methyl-(5-acetamido-7,8,9-tri-O-acetyl-5-N,4-O-carbonyl-3,5-dideoxy-D-glycero-α-D-galacto-non-2-ulopyranosyl]onate)-β-D-galactopyranosyl}-3-O-(2,3,4-tri-O-benzyl-α-L-fucopyranosyl)-6-O-benzyl-2-deoxy-2-(2,2,2-trichlorethoxy-carbonylamino)-β-D-glucopyranosyl-L-threonine tert-butyl ester (31)

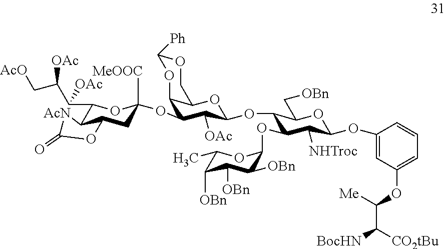

Sialyl Lewis^{X} imidate 30 (100 mg, 0.057 mmol, 1 equiv) and 23 (29 mg, 0.080 mmol, 1.4 equiv) were dissolved in dichloromethane with activated 4 Å molecular sieves for 1 hour at rt. The reaction mixture was cooled to −10° C. and TMSOTf (2 μL, 0.011 mmol, 0.2 equiv) was slowly added and stirred for 1 hour. The reaction was quenched with triethylamine (0.2 mL) and the reaction mixture diluted in dichloromethane was filtered through Celite (5 g), washed with sodium bicarbonate (200 mL), brine (200 mL) and dried with anhydrous Na₂SO₄. Solvents were removed in vacuo and the crude mixture was subjected to flash chromatography, which gave the purified 31 as a white foam in 87% yield. R_{f}=0.43 (n-hexanes:ethyl acetate=1/1). ¹H NMR (600 MHz, CDCl₃): δ 7.63 (d, J=7.72 Hz, 1H), 7.42-7.19 (m, 21H), 7.18 (d, J=7.72 Hz, 1H), 7.09 (app. t., J=8.54 Hz, 1H), 6.61 (d, J=8.54 Hz, 1H), 6.58-6.55 (m, 2H), 5.70-5.65 (m, 1H), 5.59-5.52 (m, 2H), 5.48 (s, 1H), 5.45-5.42 (m, 1H), 5.35 (d, J=9.67 Hz, 1H), 5.31-5.25 (m, 1H), 5.12-5.08 (m, 1H), 4.90-4.86 (m, 1H), 4.85-4.77 (m, 3H), 4.76-4.60 (m, 5H), 4.52-4.45 (m, 3H), 4.41-4.34 (m, 3H), 4.33-4.27 (m, 1H), 4.23 (d, J=10.95 Hz, 1H), 4.18-4.13 (m, 1H), 4.07 (app. t., J=9.02 Hz, 1H), 4.03-3.84 (m, 7H), 3.72 (app. t., J=9.67 Hz, 1H), 3.63-3.59 (m, 1H), 3.58 (s, 3H), 3.55 (app. t., J=5.16 Hz, 1H), 3.49-3.46 (m, 1H), 3.22 (m, 1H), 3.11 (dd, J=12.24, 3.87 Hz, 1H), 2.51 (s, 3H), 2.16 (s, 6H), 2.07 (s, 3H), 2.01 (app. t., J=12.24 Hz, 1H), 1.97 (s, 3H), 1.51 (s, 9H), 1.35 (s, 9H), 1.29 (d, J=5.80 Hz, 1H), 1.18 (d, J=7.09 Hz, 1H). ¹³C NMR (150 MHz, CDCl₃): δ 171.9, 170.7, 170.3, 169.7, 169.3, 168.8, 168.5, 168.4, 158.4, 158.3, 156.2, 153.5, 153.4, 139.6, 139.5, 138.7, 138.2, 137.9 129.8, 1228.9, 128.4, 128.3, 128.2, 127.9, 127.6, 127.4, 127.3, 127.1, 126.9, 126.0, 125.9, 110.1, 109.9, 105.7, 99.82, 99.71, 99.56, 98.09, 97.87, 97.72, 97.28, 95.39, 94.71, 82.28, 79.91, 79.25, 78.91, 76.02, 75.62, 75.17, 75.15, 75.08, 74.89, 74.51, 74.36, 74.11, 73.41, 73.28, 73.18, 72.66, 71.83, 71.79, 71.54, 69.51, 69.15, 68.17, 67.98, 66.50, 66.29, 63.26, 59.48, 59.122, 58.1953.23, 37.14, 28.40, 27.94, 24.69, 21.32, 20.97, 20.89, 20.75, 16.11, 15.99.

### N-(tert-butoxycarbonyl)-O-3-(phenoxy)-[4-O-{2-O-acetyl-4,6-benzylidene-3-O-[methyl-(5-acetamido-7,8,9-tri-O-acetyl-5-N,4-O-carbonyl-3,5-dideoxy-D-glycero-α-D-galacto-non-2-ulopyranosyl]onate)-β-D-galactopyranosyl}-3-O-(2,3,4-tri-O-benzyl-α-L-fucopyranosyl)-6-O-benzyl-2-acetamido-2-deoxy)-β-D-glucopyranosyl-L-threonine tert-butyl ester (32)

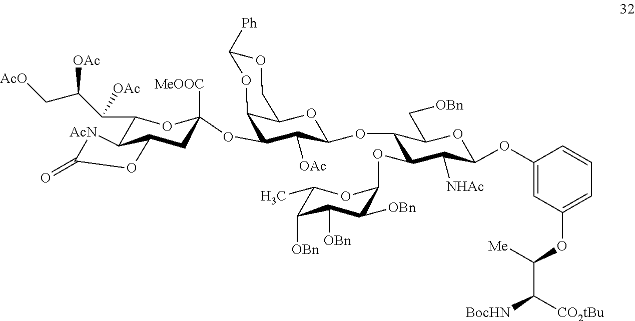

Compound 31 (97 mg, 0.053 mmol, 1 equiv) was dissolved in a solvent mixture containing tetrahydrofuran, acetic anhydride, and acetic acid (v/v 10:10:1). Zinc (212 mg) was then added and vigorously stirred for 1 hour at rt. Upon completion, zinc was filtered through Celite (5 g) and the solvent mixture was co-evaporated with toluene thrice. The crude material was subjected to flash column chromatography, which gave the purified 32 as a white foam.

### N-(tert-butoxycarbonyl)-O-3-(phenoxy)-[4-O-{2,4,6-tri-O-acetyl-3-O-[methyl-(5-acetamido-7,8,9-tri-O-acetyl-5-N,4-O-carbonyl-3,5-dideoxy-D-glycero-α-D-galacto-non-2-ulopyranosyl]onate)-β-D-galactopyranosyl}-3-O-(2,3,4-tri-O-acetyl-α-L-fucopyranosyl)-6-O-acetyl-2-acetamido-2-deoxy)-β-D-glucopyranosyl-L-threonine tert-butyl ester (33)

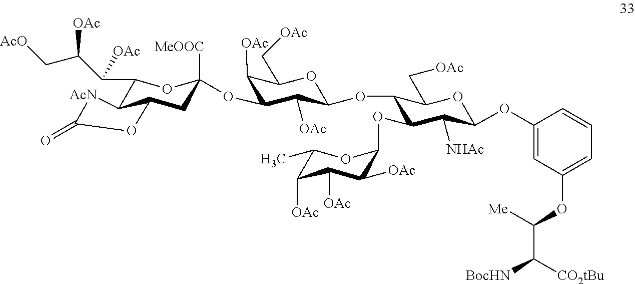

Compound 32 (69 mg, 0.038 mmol, 1 equiv) was dissolved in methanol and palladium (100 mg) was added under hydrogen gas (1 atm) for 17 hours. The crude mixture was filtered through Celite (2 g) and solvents were removed in vacuo and an azeotrope was formed with toluene thrice, which was removed in vacuo. The crude mixture was then dissolved in pyridine (10 mL) at 0° C. and acetic anhydride (5 mL) was added and stirred overnight. After 17 hours, the reaction was diluted in ethyl acetate (250 mL), washed with water (250 mL), 1 N HCl (250 mL) thrice, saturated sodium bicarbonate solution (250 mL), and brine (250 mL). The organic layer was collected, dried over Na₂SO₄ and the solvents were removed in vacuo. Purification via flash column chromatography gave 33 as a white foam.

### N-(Fluoren-9-ylmethoxycarbonyl)-O-3-(phenoxy)-[4-O-{2,4,6-tri-O-acetyl-3-O-[methyl-(5-acetamido-7,8,9-tri-O-acetyl-5-N,4-O-carbonyl-3,5-dideoxy-D-glycero-α-D-galacto-non-2-ulopyranosyl]onate)-β-D-galactopyranosyl}-3-O-(2,3,4-tri-O-acetyl-α-L-fucopyranosyl)-6-O-acetyl-2-acetamido-2-deoxy)-β-D-glucopyranosyl-L-threonine tert-butyl ester (34)

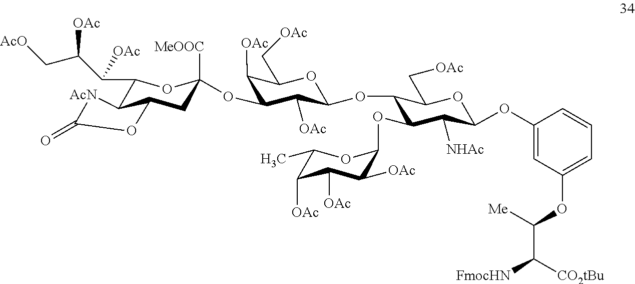

Compound 33 (49 mg, 0.028 mmol, 1 equiv) was dissolved in 1,4-dioxane (10 mL) and 1 N HCl in 1,4-dioxane (5 mL) was added and let stir for 30 minutes. Then, 1,4-dioxane was removed in vacuo and an azeotrope was formed with toluene thrice, which was then removed in vacuo. The crude mixture was then re-constituted in 1,4-dioxane (5 mL) and water (5 mL). At 0° C., sodium bicarbonate (6 mg, 0.070 mmol, 2.5 equiv) was added slowly and stirred for 30 minutes. Then, Fmoc-OSu (11 mg, 0.034 mmol, 1.2 equiv) was slowly added and stirred for 17 hours. The reaction was then diluted in ethyl acetate (125 mL), washed with brine (125 mL), and dried over Na₂SO₄. The solvents were removed in vacuo and purification via flash column chromatography gave 34 as a white foam.

### N-(Fluoren-9-ylmethoxycarbonyl)-O-3-(phenoxy)-[4-O-{2,4,6-tri-O-acetyl-3-O-[methyl-(5-acetamido-7,8,9-tri-O-acetyl-5-N,4-O-carbonyl-3,5-dideoxy-D-glycero-α-D-galacto-non-2-ulopyranosyl]onate)-β-D-galactopyranosyl}-3-O-(2,3,4-tri-O-acetyl-α-L-fucopyranosyl)-6-O-acetyl-2-acetamido-2-deoxy)-β-D-glucopyranosyl-L-threonine (35)

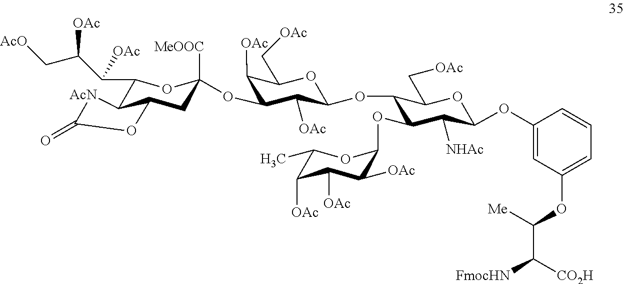

At 0° C., compound 34 (37 mg, 0.021 mmol, 1 equiv) was dissolved in dichloromethane (10 mL). Triethylsilane (0.7 μL, 0.004 mmol, 0.2 equiv) and trifluoroacetic acid (5 mL) were added dropwise, respectively. The reaction mixture was slowly warmed to rt, stirred for 3 h, and concentrated in vacuo to afford a yellow oil, which was diluted in ethyl acetate and washed carefully with sodium bicarbonate then 1 N HCl to acidify the crude mixture to a pH at ˜1-1.5, from which the crude mixture was extracted in ethyl acetate. The organic layer was washed with brine then dried with anhydrous Na₂SO₄. The solvents were dried in vacuo then the resulting mixture was subjected to flash chromatography, which gave the purified 35 as a yellow oil.

### Experimental Procedures for Solid Phase Peptide Synthesis of Aryl GSnPs

Dimethylformamide was used to swell the Novasyn TGA resin (0.18 mmol loading capacity), which was loaded into a propylene centrifuge filter tube (0.22 μM micron) for 20 minutes at rt. The coupling reactions were performed using Fmoc protected amino acids (0.5 M in DMF, 5 equiv), HBTU (0.5 M in DMF, 4.99 equiv), and DIPEA (0.5 M in DMF, 12.5 equiv) and shaken for 2 hours at rt. During the Fmoc Phe (TCE-SO₃)—OH coupling (Fs3, Fs5, and Fs8), the reaction times were increased to 4 hours at rt. Fmoc cleavage was performed using 20% piperidine in DMF for 8 minutes twice at rt and washed with DMF six times at rt. Cleavage from the resin was performed using TFA/TIPS/H₂O (95/2.5/2.5) cocktail and the resin was filtered off and washed with TFA twice. The collected TFA liquids were removed and cold diethyl ether was used to obtain the precipitate, which was centrifuged twice and collected.

### Deprotection Protocols for Aryl GSnP-1

The acetyl groups of the aryl GSnP-1 were removed using 1 N LiOH (44 equiv) in 1:1 mixture of methanol/water (0.1 M) for 30 minutes at rt. Upon completion, the reaction was quenched with acetic acid. Subsequently, a stoichiometric amount of zinc (4× mmol) was added and the reaction proceeded for 1 hour at rt to remove the resulting TCE groups. The zinc solids were filtered off and washed twice with water/methanol before RP-HPLC (Method A) purification and lyophilized to obtain the desired aryl GSnP-1 in 28% yield. HRMS: (ESI-MS m/z) calc. mass for [C₁₂₁H₁₆₆N₁₉O₄₇S₃—H⁺] 2733.0346, found 2733.043.

### Deprotection Protocols for Aryl GSnP-4

The TCE groups of the protected aryl GSnP-4 (1 equiv) were removed using zinc (4× mmol) and acetic acid (4 equiv) in methanol/water mixture (v/v 1:1) at 0° C. The reaction was slowly warmed to rt and let stir for 1 hour. At 0° C., 1 N LiOH (44 equiv) was added carefully to retain the pH at approximately 10. After 30 minutes, the reaction was neutralized with acetic acid and subjected to RP-HPLC (Method B) purification and lyophilized to afford the desired aryl GSnP-4.

### HPLC Purification Methods

### Method A: For Aryl GSnP-1

Solvent A=Water+0.1% TFA
Solvent B=Acetonitrile

| Time | Flow | | |
|---|---|---|---|
| (mins) | (mL/min) | % A | % B |
| 2 | 40 | 95 | 5 |
| 5 | 40 | 80 | 20 |
| 16 | 40 | 55 | 45 |
| 17 | 40 | 2 | 98 |
| 18.5 | 40 | 2 | 98 |
| 20 | 40 | 95 | 5 |

### Method B: For Aryl GSnP-4

Solvent A=95% Water+5% Acetonitrile+0.1% TFA
Solvent B=5% Water+95% Acetonitrile+0.1% TFA

| Time | Flow | | |
|---|---|---|---|
| (mins) | (mL/min) | % A | % B |
| 2 | 2.5 | 95 | 5 |
| 22 | 2.5 | 85 | 15 |
| 100 | 2.5 | 75 | 25 |
| 118 | 2.5 | 5 | 90 |
| 120 | 2.5 | 5 | 95 |

### Experimental Procedure for Chemoenzymatic Synthesis of Aryl GSnP-4

Aryl GSnP-1 was subjected to sodium cacodylate buffer (pH 6.5, 1.7 mM) and MgCl₂, UDP-galactose, and β4GALT1 were added and incubated at 37° C. for 18 hours. Upon completion, CMP-sialic acid and ST3GAL4 were added and incubated for 18 hours followed by the addition of GDP-fucose and FUT7, which was incubated for an additional 18 hours. Subsequently, 0.1% TFA was added and the crude reaction mixture was purified by HPLC (using Method B) and lyophilized to obtain the desired aryl GSnP-4 in 86% yield. HRMS: (ESI-MS m/z) calc. mass for [C₁₄₄H₂₀₃N₂₀O₆₄S₃—H⁺] 3332.2407, found 3332.2540.

### Experimental Procedure for Biotinylated Aryl GSnP-4

(±)-Biotin N-hydroxysuccinimide ester (20 equiv) was added to aryl GSnP-4 (1.3 mg) submerged in DMF (2 mL). Hunig's base (12.5 equiv) was added carefully and the reaction was shaken for 24 hours at rt, after which acetic acid was used to quench the reaction. The resulting solution was lyophilized and subjected directly to HPLC purification (using Method A). Biotinylated aryl GSnP-4 was obtained in 61% yield. HRMS: (MALDI-TOF m/z) calc. mass for [C₁₄₄H₂₀₃N₂₀O₆₄S₃-C₉H₁₃N₂O₂S-Na⁺-2H⁺] 3354.2227, found 3355.8630.

### Biacore Data Set

Dissociation constants (Kd) were determined using a Biacore binding assay after initial capture of biotinylated ligands onto streptavidin-coated sensor chips followed by flow through of increasing concentrations of P-selectin-Ig (0.4, 1.2, 3.6, 11, 33, 100 nM). The Biacore assay demonstrated strong binding of G4 to human P-selectin at a Kd of 50 nM, which was similar to native PSGL-1 and GSnP-6. A Biacore assay demonstrated strong binding of aryl-GSnP-4 to human P-selectin at a Kd of 37 nM (Table 2).

TABLE 2
Dissociation Constants for Core 2 Mimics

| Peptide | Kd (nM) |
|---|---|
| Click-GSP-4 | 50 |
| G4 | 69 |
| Aryl-GSnP-4 | 37 |
| GSnP-6 | 27 |

### Example 3: Single Dose, Ascending PK Study of Compound P-G4 (Pegylated Click Compound)

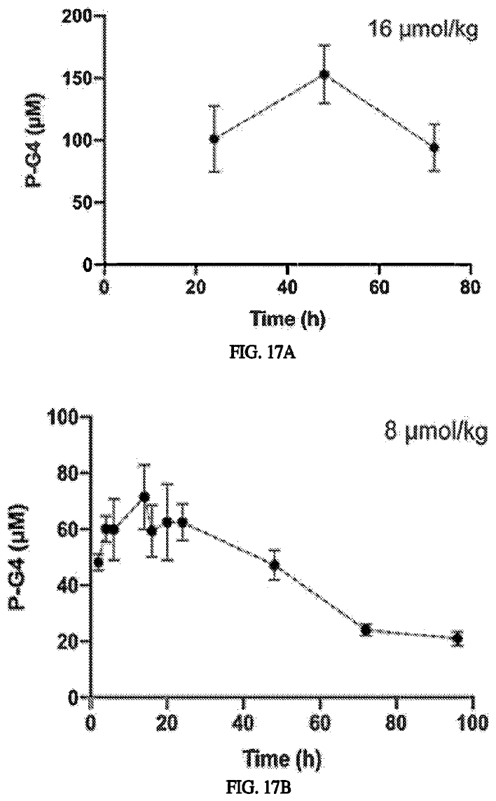

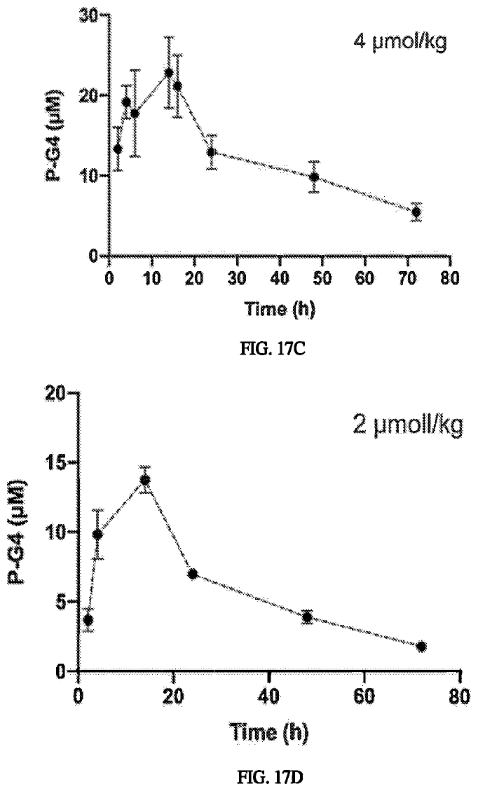

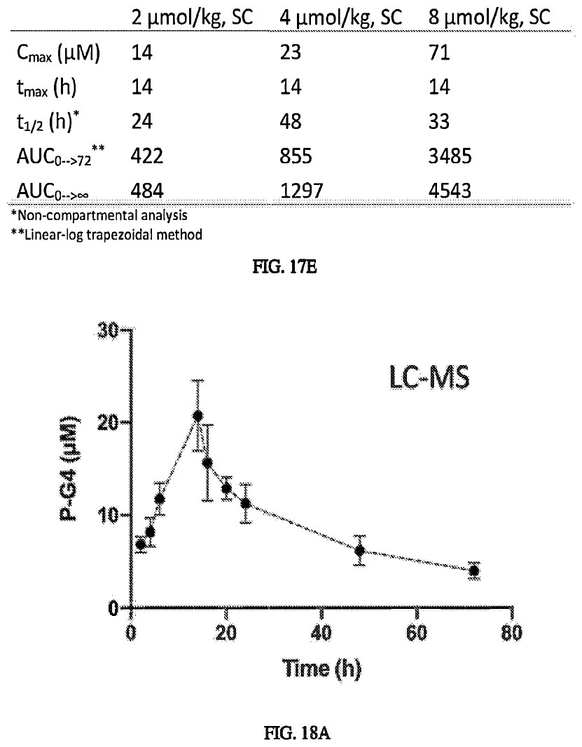

P-G4 was administered as a single subcutaneous dose in mice (n=4 mice/time point), and plasma concentrations were measured using an ELISA assay. An initial trial was performed at 16 μmol/kg to determine if the compound could be measured in plasma (FIG. 17A). For 4 and 8 μmol/kg subcutaneous doses, plasma concentrations were determined at 2, 4, 6, 14, 16, 20, 24, 48, and 72 h (FIGS. 17B and 17C, respectively). For a 2 μmol/kg subcutaneous dose, plasma concentrations were determined at 2, 4, 14, 24, 48, and 72 h (FIG. 17D). Pharmacokinetic parameters were calculated for subcutaneous delivery of P-G4 (FIG. 17E). FIGS. 18A and 18B show the correlation of ELISA and LC/MS measurements for plasma concentrations after subcutaneous delivery at 4 μmol/kg.

### Example 4: Inhibition of Venous Thrombus Formation after Subcutaneous Administration of P-G4: Murine Model of Nonocclusive Venous Thrombosis

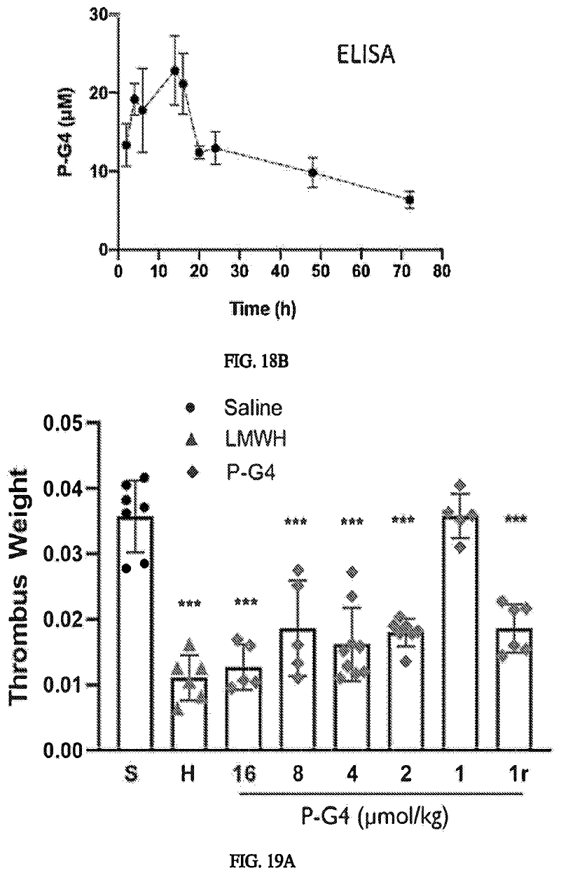

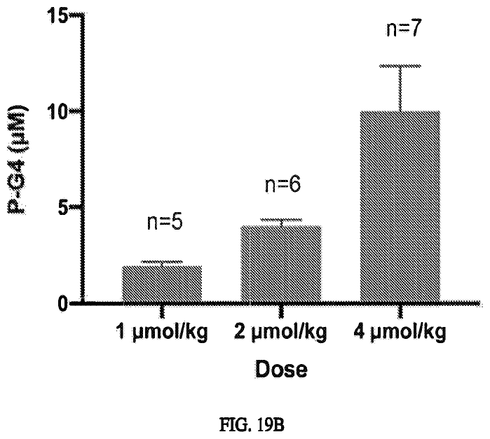

Drug efficacy after subcutaneous administration was evaluated in a preclinical mouse model in which nonocclusive venous thrombosis was induced by electrolytic injury of the inferior vena cava. Low molecular weight heparin (enoxaparin, H) (6 mg/kg), saline, or P-G4 were administered subcutaneously (8, 4, 2, or 1 μmol/kg) to male C57BL/6 mice (8-12 weeks of age) 4 hours prior and 24 hours after electrolytic injury. The vena cava and associated thrombus, immediately below the renal veins to just above the bifurcation, were excised 48 hours after injury for determination of wet thrombus weight (FIG. 19A). Plasma concentration of P-G4 was determined at the time of thrombus harvest in the two-dose protocol (FIG. 19B).

In an additional dosing protocol, P-G4 was administered at 16 μmol/kg as a single SC dose, 48 hours before electrolytic injury. The vena cava and associated thrombus was subsequently excised 48 hours after injury for determination of wet thrombus weight (FIG. 19A).

In an additional dosing protocol, P-G4 was administered as a once daily SC dose at 1 μmol/kg for 4 doses, followed by electrolytic injury, and a subsequent dose at 1 μmol/kg 24 hours later. The vena cava and associated thrombus was subsequently excised 48 hours after injury for determination of wet thrombus weight (FIG. 19A, cohort 1r).

The electrolytic injury of the inferior vena cava is conducted as follows. C57BL/6 mice were anesthetized with 2% isoflurane, and the inferior vena cava was approached via a midline laparotomy. Venous side branches were ligated or cauterized, whereas posterior branches were left patent. A 25-gauge stainless steel needle, attached to a silver-coated copper wire, was inserted into the exposed caudal vena cava and positioned against the anterior wall (anode). A second wire was implanted subcutaneously to complete the circuit (cathode), and a 250 microamp current was applied for 15 minutes. Subsequently, the needle was removed, and a cotton swab was held in gentle contact with the puncture site to prevent bleeding. The vena cava and associated thrombus, immediately below the renal veins to just above the bifurcation, were excised 48 hours after injury for determination of wet thrombus weight and histological examination.

### REFERENCES

1. Khorana A A, Francis C W, Culakova E, Kuderer N M, Lyman G H. Thromboembolism is a leading cause of death in cancer patients receiving outpatient chemotherapy. J Thromb Haemost 2007; 5(3):632-4.
2. Blom J W, Doggen C J, Osanto S, Rosendaal F R. Malignancies, prothrombotic mutations, and the risk of venous thrombosis. JAMA 2005; 293(6):715-22.
3. Walker A J, Card T R, West J, Crooks C, Grainge M J. Incidence of venous thromboembolism in patients with cancer—a cohort study using linked United Kingdom databases. Eur J Cancer 2013; 49(6):1404-13.
4. Jara-Palomares L, Solier-Lopez A, Elias-Hernandez T, Asensio-Cruz M, Blasco-Esquivias I, Marin-Barrera L, de la Borbolla-Artacho M R, Praena-Fernandez J M, Montero-Romero E, Navarro-Herrero S, Serrano-Gotarredona M P, Sanchez-Diaz J M, Palacios C, Otero R. Tinzaparin in cancer associated thrombosis beyond 6 months: TiCAT study. Thromb Res 2017; 157:90-6.
5. Roopkumar J, Kim A S, Bicky T, Hobbs B P, Khorana A A. Venous thromboembolism in cancer patients receiving immunotherapy. Blood 2018; 132 (Supplement 1):2510.
6. Prandoni P, Lensing A W, Piccioli A, Bernardi E, Simioni P, Girolami B, Marchiori A, Sabbion P, Prins M H, Noventa F, Girolami A. Recurrent venous thromboembolism and bleeding complications during anticoagulant treatment in patients with cancer and venous thrombosis. Blood 2002; 100(10):3484-8.
7. Monreal M, Falga C, Valdes M, Suarez C, Gabriel F, Tolosa C, Montes J, Riete I. Fatal pulmonary embolism and fatal bleeding in cancer patients with venous thromboembolism: Findings from the RIETE registry. J Thromb Haemost 2006; 4(9):1950-6.
8. Khorana A A, Soff G A, Kakkar A K, Vadhan-Raj S, Riess H, Wun T, Streiff M B, Garcia D A, Liebman H A, Belani C P, O'Reilly E M, Patel J N, Yimer H A, Wildgoose P, Burton P, Vijapurkar U, Kaul S, Eikelboom J, McBane R, Bauer K A, Kuderer N M, Lyman G H, Investigators C. Rivaroxaban for thromboprophylaxis in high-risk ambulatory patients with cancer. N Engl J Med 2019; 380(8):720-8.
9. Carrier M, Abou-Nassar K, Mallick R, Tagalakis V, Shivakumar S, Schattner A, Kuruvilla P, Hill D, Spadafora S, Marquis K, Trinkaus M, Tomiak A, Lee A Y Y, Gross P L, Lazo-Langner A, El-Maraghi R, Goss G, Le Gal G, Stewart D, Ramsay T, Rodger M, Witham D, Wells P S, Investigators A. Apixaban to prevent venous thromboembolism in patients with cancer. N Engl J Med 2019; 380(8):711-9.
10. Khorana A A, Francis C W, Culakova E, Kuderer N M, Lyman G H. Frequency, risk factors, and trends for venous thromboembolism among hospitalized cancer patients. Cancer 2007; 110(10):2339-46.
11. Khorana A A, Kuderer N M, Culakova E, Lyman G H, Francis C W. Development and validation of a predictive model for chemotherapy-associated thrombosis. Blood 2008; 111(10):4902-7.
12. Levitan N, Dowlati A, Remick S C, Tahsildar H I, Sivinski L D, Beyth R, Rimm A A.

Rates of initial and recurrent thromboembolic disease among patients with malignancy versus those without malignancy. Risk analysis using Medicare claims data. Medicine (Baltimore) 1999; 78(5):285-91.
13. Lyman G H, Khorana A A, Kuderer N M, Lee A Y, Arcelus J I, Balaban E P, Clarke J M, Flowers C R, Francis C W, Gates L E, Kakkar A K, Key N S, Levine M N, Liebman H A, Tempero M A, Wong S L, Prestrud A A, Falanga A, American Society of Clinical Oncology Clinical P. Venous thromboembolism prophylaxis and treatment in patients with cancer: American Society of Clinical Oncology clinical practice guideline update. J Clin Oncol 2013; 31(17):2189-204.
14. Streiff M B, Holmstrom B, Angelini D, Ashrani A, Bockenstedt P L, Chesney C, Fanikos J, Fenninger R B, Fogerty A E, Gao S, Goldhaber S Z, Gundabolu K, Hendrie P, Lee A I, Lee J T, Mann J, McMahon B, Millenson M M, Morton C, Ortel T L, Ozair S, Paschal R, Shattil S, Siddiqi T, Smock K J, Soff G, Wang T F, Williams E, Zakarija A, Hammond L, Dwyer M A, Engh A M. NCCN Guidelines insights: Cancer-associated venous thromboembolic disease, Version 2.2018. J Natl Compr Canc Netw 2018; 16(11):1289-303.
15. Francis C W, Kessler C M, Goldhaber S Z, Kovacs M J, Monreal M, Huisman M V, Bergqvist D, Turpie A G, Ortel T L, Spyropoulos A C, Pabinger I, Kakkar A K. Treatment of venous thromboembolism in cancer patients with dalteparin for up to 12 months: The DALTECAN Study. J Thromb Haemost 2015; 13(6):1028-35.
16. Raskob G E, Buller H R, Segers A. Edoxaban for cancer-sssociated venous thromboembolism. N Engl J Med 2018; 379(1):95-6.
17. Kahale L A, Hakoum M B, Tsolakian I G, Matar C F, Terrenato I, Sperati F, Barba M, Yosuico V E, Schunemann H, Akl E A. Anticoagulation for the long-term treatment of venous thromboembolism in people with cancer. Cochrane Database Syst Rev 2018; 6:CD006650.
18. Sobieraj D M, Baker W L, Smith E, Sasiela K, Trexler S E, Kim O, Coleman C I. Anticoagulation for the treatment of cancer-associated thrombosis: A systematic review and network meta-analysis of randomized trials. Clin Appl Thromb Hemost 2018; 24(9_suppl):182S-7S.
19. Young A M, Marshall A, Thirlwall J, Chapman O, Lokare A, Hill C, Hale D, Dunn J A, Lyman G H, Hutchinson C, MacCallum P, Kakkar A, Hobbs F D R, Petrou S, Dale J, Poole C J, Maraveyas A, Levine M. Comparison of an oral factor Xa inhibitor with low molecular weight heparin in patients with cancer with venous thromboembolism: Results of a randomized trial (SELECT-D). J Clin Oncol 2018; 36(20):2017-23.
20. van Es N, Coppens M, Schulman S, Middeldorp S, Buller H R. Direct oral anticoagulants compared with vitamin K antagonists for acute venous thromboembolism: Evidence from phase 3 trials. Blood 2014; 124(12):1968-75.
21. Zakai N A, Walker R F, MacLehose R F, Adam T J, Alonso A, Lutsey P L. Impact of anticoagulant choice on hospitalized bleeding risk when treating cancer-associated venous thromboembolism. J Thromb Haemost 2018; 16(12):2403-12.
22. Nieto J A, Solano R, Ruiz-Ribo M D, Ruiz-Gimenez N, Prandoni P, Kearon C, Monreal M, Riete I. Fatal bleeding in patients receiving anticoagulant therapy for venous thromboembolism: Findings from the RIETE registry. J Thromb Haemost 2010; 8(6):1216-22.
23. Koster T, Rosendaal F R, Briet E, Vandenbroucke J P. John Hageman's factor and deep-vein thrombosis: Leiden thrombophilia Study. Br J Haematol 1994; 87(2):422-4.
24. Cushman M, O'Meara E S, Folsom A R, Heckbert S R. Coagulation factors IX through XIII and the risk of future venous thrombosis: The longitudinal investigation of thromboembolism etiology. Blood 2009; 114(14):2878-83.
25. Falati S, Liu Q, Gross P, Merrill-Skoloff G, Chou J, Vandendries E, Celi A, Croce K, Furie B C, Furie B. Accumulation of tissue factor into developing thrombi in vivo is dependent upon microparticle P-selectin glycoprotein ligand 1 and platelet P-selectin. J Exp Med 2003; 197(11):1585-98.
26. Darbousset R, Thomas G M, Mezouar S, Frere C, Bonier R, Mackman N, Renne T, Dignat-George F, Dubois C, Panicot-Dubois L. Tissue factor-positive neutrophils bind to injured endothelial wall and initiate thrombus formation. Blood 2012; 120(10):2133-43.
27. von Bruhl M L, Stark K, Steinhart A, Chandraratne S, Konrad I, Lorenz M, Khandoga A, Tirniceriu A, Coletti R, Kollnberger M, Byrne R A, Laitinen I, Walch A, Brill A, Pfeiler S, Manukyan D, Braun S, Lange P, Riegger J, Ware J, Eckart A, Haidari S, Rudelius M, Schulz C, Echtler K, Brinkmann V, Schwaiger M, Preissner K T, Wagner D D, Mackman N, Engelmann B, Massberg S. Monocytes, neutrophils, and platelets cooperate to initiate and propagate venous thrombosis in mice in vivo. J Exp Med 2012; 209(4):819-35.
28. Clark S R, Ma A C, Tavener S A, McDonald B, Goodarzi Z, Kelly M M, Patel K D, Chakrabarti S, McAvoy E, Sinclair G D, Keys E M, Allen-Vercoe E, Devinney R, Doig C J, Green F H, Kubes P. Platelet TLR4 activates neutrophil extracellular traps to ensnare bacteria in septic blood. Nat Med 2007; 13(4):463-9.
29. Ammollo C T, Semeraro F, Xu J, Esmon N L, Esmon C T. Extracellular histones increase plasma thrombin generation by impairing thrombomodulin-dependent protein C activation. J Thromb Haemost 2011; 9(9):1795-803.
30. Fuchs T A, Brill A, Duerschmied D, Schatzberg D, Monestier M, Myers D D, Jr., Wrobleski S K, Wakefield T W, Hartwig J H, Wagner D D. Extracellular DNA traps promote thrombosis. Proc Natl Acad Sci USA 2010; 107(36):15880-5.
31. Ward C M, Tetaz T J, Andrews R K, Berndt M C. Binding of the von Willebrand factor A1 domain to histone. Thromb Res 1997; 86(6):469-77.
32. Massberg S, Grahl L, von Bruehl M L, Manukyan D, Pfeiler S, Goosmann C, Brinkmann V, Lorenz M, Bidzhekov K, Khandagale A B, Konrad I, Kennerknecht E, Reges K, Holdenrieder S, Braun S, Reinhardt C, Spannagl M, Preissner K T, Engelmann B. Reciprocal coupling of coagulation and innate immunity via neutrophil serine proteases. Nat Med 2010; 16(8):887-96.
33. da Costa Martins P, van den Berk N, Ulfman L H, Koenderman L, Hordijk P L, Zwaginga J J. Platelet-monocyte complexes support monocyte adhesion to endothelium by enhancing secondary tethering and cluster formation. Arterioscler Thromb Vasc Biol 2004; 24(1):193-9.
34. Shao B, Wahrenbrock M G, Yao L, David T, Coughlin S R, Xia L, Varki A, McEver R P. Carcinoma mucins trigger reciprocal activation of platelets and neutrophils in a murine model of Trousseau syndrome. Blood 2011; 118(15):4015-23.
35. Maraveyas A, Waters J, Roy R, Fyfe D, Propper D, Lofts F, Sgouros J, Gardiner E, Wedgwood K, Ettelaie C, Bozas G. Gemcitabine versus gemcitabine plus dalteparin thromboprophylaxis in pancreatic cancer. Eur J Cancer 2012; 48(9):1283-92.
36. Ay C, Jungbauer L V, Sailer T, Tengler T, Koder S, Kaider A, Panzer S, Quehenberger P, Pabinger I, Mannhalter C. High concentrations of soluble P-selectin are associated with risk of venous thromboembolism and the P-selectin Thr715 variant. Clin Chem 2007; 53(7):1235-43.
37. Blann A D, Noteboom W M, Rosendaal F R. Increased soluble P-selectin levels following deep venous thrombosis: Cause or effect? Br J Haematol 2000; 108(1):191-3.
38. Kyrle P A, Hron G, Eichinger S, Wagner O. Circulating P-selectin and the risk of recurrent venous thromboembolism. Thromb Haemost 2007; 97(6):880-3.
39. Ay C, Simanek R, Vormittag R, Dunkler D, Alguel G, Koder S, Kornek G, Marosi C, Wagner O, Zielinski C, Pabinger I. High plasma levels of soluble P-selectin are predictive of venous thromboembolism in cancer patients: Results from the Vienna Cancer and Thrombosis Study (CATS). Blood 2008; 112(7):2703-8.
40. Ay C, Dunkler D, Marosi C, Chiriac A L, Vormittag R, Simanek R, Quehenberger P, Zielinski C, Pabinger I. Prediction of venous thromboembolism in cancer patients. Blood 2010; 116(24):5377-82.
41. Myers D, Jr., Farris D, Hawley A, Wrobleski S, Chapman A, Stoolman L, Knibbs R, Strieter R, Wakefield T. Selectins influence thrombosis in a mouse model of experimental deep venous thrombosis. J Surg Res 2002; 108(2):212-21.
42. Hrachovinova I, Cambien B, Hafezi-Moghadam A, Kappelmayer J, Camphausen R T, Widom A, Xia L, Kazazian H H, Jr., Schaub R G, McEver R P, Wagner D D. Interaction of P-selectin and PSGL-1 generates microparticles that correct hemostasis in a mouse model of hemophilia A. Nat Med 2003; 9(8):1020-5.
43. Sullivan V V, Hawley A E, Farris D M, Knipp B S, Varga A J, Wrobleski S K, Thanapron P, Eagleton M J, Myers D D, Fowlkes J B, Wakefield T W. Decrease in fibrin content of venous thrombi in selectin-deficient mice. J Surg Res 2003; 109(1):1-7.
44. Krishnamurthy V R, Sardar M Y R, Yu Y, Song X, Haller C A, Dai E, Wang X, Hanjaya-Putra D, Sun L, Morikis V, Simon S I, Woods R J, Cummings R D, Chaikof E L. Glycopeptide analogues of PSGL-1 inhibit P-selectin in vitro and in vivo. Nat Commun 2015; 6:6387. doi: 10.1038/ncomms7387.
45. Wong D J, Park D D, Park S, Haller C A, Chen J, Dai E, Liu L, Mandhapati A, Eradi P, Dhakal B, Wever W, Hanes M, Sun L, Cummings R D, Chaikof E L. A PSGL-1 glycomimetic reduces thrombus burden without affecting hemostasis Blood 2021 In press.
46. Kirschner K N, Yongye A B, Tschampel S M, Gonzalez-Outeirino J, Daniels C R, Foley B L, Woods R J. GLYCAM06: A generalizable biomolecular force field. Carbohydrates. J Comput Chem 2008; 29(4):622-55.
47. Case D A, Berryman J T, Betz R M, Cerutti D S, Cheatham III T E, Darden T A, Duke R E, Giese T J, Gohlke H, Goetz A W, Homeyer N, Izadi S, Janowski P, Kaus J, Kovalenko A, Lee T S, LeGrand S, Li P, Luchko T, Luo R, Madej B, Merz K M, Monard G, Needham P, Nguyen H, Nguyen H T, Omelyan I, Onufriev A, Roe D R, Roitberg A, Salomon-Ferrer R, Simmerling C L, Smith W, Swails J, Walker R C, Wang J, Wolf R M, Wu X, York D M, Kollman P A. AMBER 2015. University of California San Francisco 2015.
48. Frisch M J, Trucks G W, Schlegel H B, Scuseria G E, Robb M A, Cheeseman J R, Scalmani G, Barone V, Mennucci B, Petersson G A, Nakatsuji H, Caricato M, Li X, Hratchian H P, Izmaylov A F, Bloino J, Zheng G, Sonnenberg J L, Hada M, Ehara M, Toyota K, Fukuda R, Hasegawa J, Ishida M, Nakajima T, Honda Y, Kitao O, Nakai H, Vreven T, Montgomery Jr J A, Peralta J E, Ogliaro F, Bearpark M, Heyd J J, Brothers E, Kudin K N, Staroverov V N, Kobayashi R, Normand J, Raghavachari K, Rendell A, Burant J C, Iyengar S S, Tomasi J, Cossi M, Rega N, Millam J M, Klene M, Knox J E, Cross J B, Bakken V, Adamo C, Jaramillo J, Gomperts R, Stratmann R E, Yazyev O, Austin A J, Cammi R, Pomelli C, Ochterski J W, Martin R L, Morokuma K, Zakrzewski V G, Voth G A, Salvador P, Dannenberg J J, Dapprich S, Daniels A D, Farkas Ö, Foresman J B, Ortiz J V, Cioslowski J, Fox D J. Gaussian 09 Gaussian Inc, Wallingford CT 2009.
49. Jorgensen W L, Chandrasekhar J, Madura J D, Impey R W, Klein M L. Comparison of simple potential functions for simulating liquid water. J Chem Physics 1983; 79(2):926-35.
50. Berendsen H J C, Postma J P M, Vangunsteren W F, Dinola A, Haak J R. Molecular-dynamics with coupling to an external bath. J Chem Physics 1984; 81(8):3684-90.
51. Vangunsteren W F, Berendsen H J C. Algorithms for macromolecular dynamics and constraint dynamics. Molecular Physics 1977; 34(5):1311-27.
52. Gotz A W, Williamson M J, Xu D, Poole D, Le Grand S, Walker R C. Routine microsecond molecular dynamics simulations with AMBER on GPUs. 1. Generalized Born. J Chem Theory Comput 2012; 8(5):1542-55.
53. Kollman P A, Massova I, Reyes C, Kuhn B, Huo S, Chong L, Lee M, Lee T, Duan Y, Wang W, Donini O, Cieplak P, Srinivasan J, Case D A, Cheatham T E, 3rd. Calculating structures and free energies of complex molecules: Combining molecular mechanics and continuum models. Acc Chem Res 2000; 33(12):889-97.
54. Schlitter J. Estimation of absolute and relative entropies of macromolecules using the covariance-matrix. Chem Phys Lett 1993; 215(6):617-21.
55. Roe D R, Cheatham T E, 3rd. PTRAJ and CPPTRAJ: Software for processing and analysis of molecular dynamics trajectory data. J Chem Theory Comput 2013; 9(7):3084-95.
56. Karplus M, Kushick J N. Method for estimating the configurational entropy of macromolecules. Macromolecules 1981; 14(2):325-32.
57. Henseler A, Ayats C, Pericas M. An enatioselective recyclable polystyrene-supported threonine-derived organocatalyst for Aldol reactions. Adv Synth Catal 2014; 356:1795-802.
58. Hanjaya-Putra D, Haller C, Wang X, Dai E, Lim B, Liu L, Jaminet P, Yao J, Searle A, Bonnard T, Hagemeyer C, Peter K, Chaikof E. Platelet-targeted dual pathway antithrombotic inhibits thrombosis with preserved hemostasis. JCI Insight 2018; 3(15):e99329.
59. Leppanen A, White S P, Helin J, McEver R P, Cummings R D. Binding of glycosulfopeptides to P-selectin requires stereospecific contributions of individual tyrosine sulfate and sugar residues. J Biol Chem 2000; 275(50):39569-78.
60. Thirumurugan P, Matosiuk D, Jozwiak K. Click chemistry for drug development and diverse chemical-biology applications. Chem Rev 2013; 113(7):4905-79.
61. Sood A, Gerlits O O, Ji Y, Bovin N V, Coates L, Woods R J. Defining the specificity of carbohydrate-protein interactions by quantifying functional group contributions. J Chem Inf Model 2018; 58(9):1889-901.
62. Soriano del Amo D, Wang W, Besanceney C, Zheng T, He Y, Gerwe B, Seidel R D, 3rd, Wu P. Chemoenzymatic synthesis of the sialyl Lewis X glycan and its derivatives. Carbohydr Res 2010; 345(9):1107-13.
63. Boltje T J, Buskas T, Boons G J. Opportunities and challenges in synthetic oligosaccharide and glycoconjugate research. Nat Chem 2009; 1(8):611-22.
64. Chao Q, Ding Y, Chen Z H, Xiang M H, Wang N, Gao X D. Recent progress in chemoenzymatic methods for the synthesis of N-glycans. Front Chem 2020; 8:513.
65. Sardar M Y R, Mandhapati A R, Park S, Wever W J, Cummings R D, Chaikof E L.

Convergent synthesis of sialyl Lewis(X)-O-core-1 threonine. J Org Chem 2018; 83(9):4963-72.
66. Herzner H, Kunz H. Spacer-separated sialyl Lewis^{X} cyclopeptide conjugates as potential E-selectin ligands. Carbohydr Res 2007; 342(3-4):541-57.
67. Cao H, Huang S, Cheng J, Li Y, Muthana S, Son B, Chen X. Chemical preparation of sialyl Lewis x using an enzymatically synthesized sialoside building block. Carbohydr Res 2008; 343(17):2863-9.
68. Lu D, Hu Y, He X, Sollogoub M, Zhang Y. Total synthesis of a sialyl Lewis(x) derivative for the diagnosis of cancer. Carbohydr Res 2014; 383:89-96.
69. Bernet B, Vasella A, Liu Q, Tor Y. In: Charette A, Bode J, Rovis T, and Shenvi R eds. Encyclopedia of Reagents for Organic Synthesis. 2009.
70. Smid P, de Ruiter G A, van der Marel G A, Rombouts F M, van Boom J H. Iodonium-Ion assisted stereospecific glycosylation: Synthesis of oligosaccharides containing α(1→4)-linked L-fucopyranosyl units. J Carbohydr Chem 1991; 10:833-49.
71. Hernindez-Torres J M, Liew S-T, Achkar J, Wei A. Optimized synthesis of an orthogonally protected glucosamine. Synthesis 2002; 4:487-90.
72. Mukherjee A, Palcic M M, Hindsgaul O. Synthesis and enzymatic evaluation of modified acceptors of recombinant blood group A and B glycosyltransferases. Carbohydr Res 2000; 326(1):1-21.
73. Li Z, Gildersleeve J C. Mechanistic studies and methods to prevent aglycon transfer of thioglycosides. J Am Chem Soc 2006; 128(35):11612-9.
74. Crich D, Li W. O-sialylation with N-acetyl-5-n,4-o-carbonyl-protected thiosialoside donors in dichloromethane: Facile and selective cleavage of the oxazolidinone ring. J Org Chem 2007; 72(7):2387-91.
75. Hsu C H, Chu K C, Lin Y S, Han J L, Peng Y S, Ren C T, Wu C Y, Wong C H. Highly alpha-selective sialyl phosphate donors for efficient preparation of natural sialosides. Chemistry 2010; 16(6):1754-60.
76. Schmidt R R, Zimmermann P. Synthesis of glycosphingolipids and psychosines. Angew Chem Int Ed 1986; 25:725-6.
77. Baumann K, Kowalczyk D, Kunz H. Total synthesis of the glycopeptide recognition domain of the P-selectin glycoprotein ligand 1. Angew Chem Int Ed Engl 2008; 47(18):3445-9.
78. Sardar M Y R, Krishnamurthy V R, Park S, Mandhapati A R, Wever W J, Park D, Cummings R D, Chaikof E L. Synthesis of Lewis(X)—O-Core-1 threonine: A building block for O-linked Lewis(X) glycopeptides. Carbohydr Res 2017; 452:47-53.
79. Schmidt R R, Kinzy W. Anomeric-oxygen activation for glycoside synthesis: The trichloroacetimidate method. Adv Carbohydr Chem Biochem 1994; 50:21-123.
80. Meldal M, Tornoe C W. Cu-catalyzed azide-alkyne cycloaddition. Chem Rev 2008; 108(8):2952-3015.
81. Hsieh H W, Davis R A, Hoch J A, Gervay-Hague J. Two-step functionalization of oligosaccharides using glycosyl iodide and trimethylene oxide and its applications to multivalent glycoconjugates. Chemistry 2014; 20(21):6444-54.
82. Fantoni N Z, El-Sagheer A H, Brown T. A hitchhiker's guide to click-chemistry with nucleic acids. Chem Rev 2021; doi: 10.1021/acs.chemrev.0c00928.
83. Harris J M, Chess R B. Effect of pegylation on pharmaceuticals. Nat Rev Drug Discov 2003; 2(3):214-21.
84. Li F, Erickson H P, James J A, Moore K L, Cummings R D, McEver R P. Visualization of P-selectin glycoprotein ligand-1 as a highly extended molecule and mapping of protein epitopes for monoclonal antibodies. J Biol Chem 1996; 271(11):6342-8.
85. Mehta P, Cummings R D, McEver R P. Affinity and kinetic analysis of P-selectin binding to P-selectin glycoprotein ligand-1. J Biol Chem 1998; 273(49):32506-13.
86. Evangelista V, Manarini S, Sideri R, Rotondo S, Martelli N, Piccoli A, Totani L, Piccardoni P, Vestweber D, de Gaetano G, Cerletti C. Platelet/polymorphonuclear leukocyte interaction: P-selectin triggers protein-tyrosine phosphorylation-dependent CD11b/CD18 adhesion: Role of PSGL-1 as a signaling molecule. Blood 1999; 93(3):876-85.
87. Etulain J, Martinod K, Wong S L, Cifuni S M, Schattner M, Wagner D D. P-selectin promotes neutrophil extracellular trap formation in mice. Blood 2015; 126(2):242-6.
88. Leppanen A, Mehta P, Ouyang Y-B, Ju T, Helin J, Moore K L, Van Die I, Canfield W M, McEver R P, Cummings R D. A novel glycosulfopeptide binds to P-selectin and inhibits leukocyte adhesion to P-selectin. J Biol Chem 1999; 274(35):24838-48.
89. Chang J, Patton J T, Sarkar A, Ernst B, Magnani J L, Frenette P S. GMI-1070, a novel pan-selectin antagonist, reverses acute vascular occlusions in sickle cell mice. Blood 2010; 116(10):1779-86.
90. Huang A, Moretto A, Janz K, Lowe M, Bedard P W, Tam S, Di L, Clerin V, Sushkova N, Tchernychev B, Tsao D H, Keith J C, Shaw G D, Schaub R G, Wang Q, Kaila N. Discovery of 2-[1-(4-chlorophenyl)cyclopropyl]-3-hydroxy-8-(trifluoromethyl)quinoline-4-carboxylic acid (PSI-421), a P-selectin inhibitor with improved pharmacokinetic properties and oral efficacy in models of vascular injury. J Med Chem 2010; 53(16):6003-17.
91. Kaila N, Janz K, DeBernardo S, Bedard P W, Camphausen R T, Tam S, Tsao D H H, Keith J C, Jr., Nickerson-Nutter C, Shilling A, Young-Sciame R, Wang Q. Synthesis and biological evaluation of quinoline salicylic acids as P-selectin antagonists. J Med Chem 2007; 50(1):21-39.
92. Kranich R, Busemann A S, Bock D, Schroeter-Maas S, Beyer D, Heinemann B, Meyer M, Schierhorn K, Zahlten R, Wolff G, Aydt E M. Rational design of novel, potent small molecule pan-selectin antagonists. J Med Chem 2007; 50(6):1101-15.
93. Meier T R, Myers D D, Jr., Wrobleski S K, Zajkowski P J, Hawley A E, Bedard P W, Ballard N E, Londy F J, Kaila N, Vlasuk G P, Schaub R G, Wakefield T W. Prophylactic P-selectin inhibition with PSI-421 promotes resolution of venous thrombosis without anticoagulation. Thromb Haemost 2008; 99(2):343-51.
94. Watz H, Bock D, Meyer M, Schierhorn K, Vollhardt K, Woischwill C, Pedersen F, Kirsten A, Beeh K M, Meyer-Sabellek W, Magnussen H, Beier J. Inhaled pan-selectin antagonist Bimosiamose attenuates airway inflammation in COPD. Pulm Pharmacol Ther 2013; 26(2):265-70.
95. Leppanen A, Mehta P, Ouyang Y-B, Ju T, Helin J, Moore K L, Van Die I, Canfield W M, McEver R P, Cummings R D. A novel glycosulfopeptide binds to P-selectin and inhibits leukocyte adhesion to P-selectin. J Biol Chem 1999; 274(35):24838-48.
96. Leppanen A, White S P, Helin J, McEver R P, Cummings R D. Binding of glycosulfopeptides to P-selectin requires stereospecific contributions of individual tyrosine sulfate and sugar residues. J Biol Chem 2000; 275(50):39569-78.
97. Leppanen A, Yago T, I. O V, McEver R P, Cummings R D. Model glycosulfopeptides from P-selectin glycoprotein ligand-1 require tyrosine sulfation and a core 2-branched 0-glycan to bind to L-selectin. J Biol Chem 2003; 278(29):26391-400.
98. Baumann K, Kowalczyk D, Kunz H. Total synthesis of the glycopeptide recognition domain of the P-selectin glycoprotein ligand 1. Angew Chem Int Ed 2008; 47(18):3445-9.
99. Vohra Y, Buskas T, Boons G J. Rapid assembly of oligosaccharides: A highly convergent strategy for the assembly of a glycosylated amino acid derived from PSGL-1. J Org Chem 2009; 74(16):6064-71.
100. Huang K T, Wu B C, Lin C C, Luo S C, Chen C, Wong C-H, Lin C. Multi-enzyme one-pot strategy for the synthesis of sialyl Lewis X-containing PSGL-1 glycopeptide. Carbohydr Res 2006; 341(12):2151-5.
101. Koeller K M, Smith M E B, Huang R F, Wong C H. Chemoenzymatic synthesis of a PSGL-1 N-terminal glycopeptide containing tyrosine sulfate and α-O-linked sialyl Lewis X. J Am Chem Soc 2000; 122(17):4241-2.
102. Krishnamurthy V R, Dougherty A, Kamat M, Song X, Cummings R D, Chaikof E L. Synthesis of an Fmoc-threonine bearing core-2 glycan: A building block for PSGL-1 via Fmoc-assisted solid-phase peptide synthesis. Carbohydr Res 2010; 345(11):1541-7.
103. Chaikof E L, Cummings R D. Selectin inhibitors, composition, and uses related thereto. U.S. Pat. No. 10,253,071: Apr. 9, 2019.
104. Eppihimer M J, Schaub R G. P-selectin-dependent inhibition of thrombosis during venous stasis. Arterioscler Thromb Vasc Biol 2000; 20(11):2483-8.
105. Wakefield T W, Strieter R M, Schaub R, Myers D D, Prince M R, Wrobleski S K, Londy F J, Kadell A M, Brown S L, Henke P K, Greenfield U. Venous thrombosis prophylaxis by inflammatory inhibition without anticoagulation therapy. Journal of Vascular Surgery 2000; 31(2):309-24.
106. Thanaporn P, Myers D D, Wrobleski S K, Hawley A E, Farris D M, Wakefield T W, Henke P K. P-selectin inhibition decreases post-thrombotic vein wall fibrosis in a rat model.

Surgery 2003; 134(2):365-71.
107. Downing L J, Wakefield T W, Strieter R M, Prince M R, Londy F J, Fowlkes J B, Hulin M S, Kadell A M, Wilke C A, Brown S L, Wrobleski S K, Burdick M D, Anderson D C, Greenfield L J, Baxter T B. Anti-P-selectin antibody decreases inflammation and thrombus formation in venous thrombosis. J Vasc Surg 1997; 25(5):816-28.
108. Myers D D, Hawley A E, Farris D M, Wrobleski S K, Thanaporn P, Schaub R G, Wagner D D, Kumar A, Wakefield T W. P-selectin and leukocyte microparticles are associated with venous thrombogenesis. J Vasc Surg 2003; 38(5):1075-89.
109. Wakefield T W, Strieter R M, Downing L J, Kadell A M, Wilke C A, Burdick M D, Wrobleski S K, Phillips M L, Paulson J C, Anderson D C, Greenfield U. P-selectin and TNF inhibition reduce venous thrombosis inflammation. J Surg Res 1996; 64(1):26-31.
110. Myers D D, Rectenwald J E, Bedard P W, Kaila N, Shaw G D, Schaub R G, Farris D M, Hawley A E, Wrobleski S K, Henke P K, Wakefield T W. Decreased venous thrombosis with an oral inhibitor of P selectin. J Vasc Surg 2005; 42(2):329-36.
111. Myers D D, Henke P K, Bedard P W, Wrobleski S K, Kaila N, Shaw G, Meier T R, Hawley A E, Schaub R G, Wakefield T W. Treatment with an oral small molecule inhibitor of P selectin (PSI-697) decreases vein wall injury in a rat stenosis model of venous thrombosis.

J Vasc Surg 2006; 44(3):625-32.
112. Myers D D, Wrobleski S K, Longo C, Bedard P W, Kaila N, Shaw G D, Londy F J, Rohrer S E, Fex B A, Zajkowski P J, Meier T R, Hawley A E, Farris D M, Ballard N E, Henke P K, Schaub R G, Wakefield T W. Resolution of venous thrombosis using a novel oral small-molecule inhibitor of P-selectin (PSI-697) without anticoagulation. Thromb Haemostasis 2007; 97(3):400-7.
113. Meier T R, Myers D D, Wrobleski S K, Zajkowski P J, Hawley A E, Bedard P W, Ballard N E, Londy F J, Kaila N, Vlasuk G P, Schaub R G, Wakefield T W. Prophylactic P-selectin inhibition with PSI-421 promotes resolution of venous thrombosis without anticoagulation. Thromb Haemostasis 2008; 99(02):343-51.
114. Diaz J A, Wrobleski S K, Alvarado C M, Hawley A E, Doornbos N K, Lester P A, Lowe S E, Gabriel J E, Roelofs K J, Henke P K, Schaub R G, Wakefield T W, Myers D D. P-Selectin inhibition therapeutically promotes thrombus resolution and prevents vein wall fibrosis better than enoxaparin and an inhibitor to von willebrand factor. Arterioscl Thromb Vasc Biol 2015; 35(4):829-37.
115. Myers D, Farris D, Hawley A, Wrobleski S, Chapman A, Stoolman L, Knibbs R, Strieter R, Wakefield T. Selectins influence thrombosis in a mouse model of experimental deep venous thrombosis. J Surg Res 2002; 108(2):212-21.
116. Falati S, Liu Q, Gross P, Merrill-Skoloff G, Chou J, Vandendries E, Celi A, Croce K, Furie B C, Furie B. Accumulation of tissue factor into developing thrombi in vivo is dependent upon microparticle P-selectin glycoprotein ligand 1 and platelet P-selectin. J Exp Med 2003; 197(11):1585-98.
117. von Bruhl M L, Stark K, Steinhart A, Chandraratne S, Konrad I, Lorenz M, Khandoga A, Tirniceriu A, Coletti R, Kollnberger M, Byrne R A, Laitinen I, Walch A, Brill A, Pfeiler S, Manukyan D, Braun S, Lange P, Riegger J, Ware J, Eckart A, Haidari S, Rudelius M, Schulz C, Echtler K, Brinkmann V, Schwaiger M, Preissner K T, Wagner D D, Mackman N, Engelmann B, Massberg S. Monocytes, neutrophils, and platelets cooperate to initiate and propagate venous thrombosis in mice in vivo. J Exp Med 2012; 209(4):819-35.
118. Sola R J, Griebenow K. Glycosylation of therapeutic proteins: An effective strategy to optimize efficacy. BioDrugs 2010; 24(1):9-21.
119. Arnold D F, Misbah S A. Cetuximab-induced anaphylaxis and IgE specific for galactose-α-1,3-galactose. N Engl J Med 2008; 358(25):2735-6.
120. Jefferis R. Glycosylation as a strategy to improve antibody-based therapeutics. Nat Rev Drug Discov 2009; 8(3):226-34.
121. Mertens P, Maes A, Nuyts J, Belmans A, Desmet W, Esplugas E, Charlier F, Figueras J, Sambuceti G, Schwaiger M, Mortelmans L, Van de Werf F, investigators P. Recombinant P-selectin glycoprotein ligand-immunoglobulin, a P-selectin antagonist, as an adjunct to thrombolysis in acute myocardial infarction. The P-Selectin Antagonist Limiting Myonecrosis (PSALM) trial. Am Heart J 2006; 152(1):125 el-8.
122. Busuttil R W, Lipshutz G S, Kupiec-Weglinski J W, Ponthieux S, Gjertson D W, Cheadle C, Watkins T, Ehrlich E, Katz E, Squiers E C, Rabb H, Hemmerich S. RPSGL-Ig for improvement of early liver allograft function: A double-blind, placebo-controlled, single-center phase II study. American Journal of Transplantation 2011; 11(4):786-97.
123. Gaber A O, Mulgaonkar S, Kahan B D, Woodle E S, Alloway R, Bajjoka I, Jensik S, Klintmalm G B, Patton P R, Wiseman A, Lipshutz G, Kupiec-Weglinski J, Gaber L W, Katz E, Irish W, Squiers E C, Hemmerich S. YSPSL (rPSGL-Ig) for improvement of early renal allograft function: A double-blind, placebo-controlled, multi-center phase IIa study. Clinical Transplantation 2011; 25(4):523-33.
124. Hossler P, Khattak S F, Li Z J. Optimal and consistent protein glycosylation in mammalian cell culture. Glycobiology 2009; 19(9):936-49.
125. Ataga K I, Kutlar A, Kanter J, Liles D, Cancado R, Friedrisch J, Guthrie T H, Knight-Madden J, Alvarez O A, Gordeuk V R, Gualandro S, Colella M P, Smith W R, Rollins S A, Stocker J W, Rother R P. Crizanlizumab for the prevention of pain crises in sickle cell disease. N Engl J Med 2017; 376(5):429-39.
126. Prado M S, Bendtzen K, Andrade L E C. Biological anti-TNF drugs: immunogenicity underlying treatment failure and adverse events. Expert Opin Drug Metab Toxicol 2017;
   13(9):985-95.
127. Harding F A, Stickler M M, Razo J, DuBridge R B. The immunogenicity of humanized and fully human antibodies: Residual immunogenicity resides in the CDR regions. mAbs 2010; 2(3):256-65.
128. Patel M S, Miranda-Nieves D, Chen J, Haller C A, Chaikof E L. Targeting P-selectin glycoprotein ligand-1/P-selectin interactions as a novel therapy for metabolic syndrome. Transl Res 2017; 183:1-13.
129. Chang J, Patton J T, Sarkar A, Ernst B, Magnani J L, Frenette P S. GMI-1070, a novel pan-selectin antagonist, reverses acute vascular occlusions in sickle cell mice. Blood 2010; 116(10):1779-86.
130. Huang A, Moretto A, Janz K, Lowe M, Bedard P W, Tam S, Di L, Clerin V, Sushkova N, Tchernychev B, Tsao D H, Keith J C, Shaw G D, Schaub R G, Wang Q, Kaila N. Discovery of 2-[1-(4-chlorophenyl)cyclopropyl]-3-hydroxy-8-(trifluoromethyl)quinoline-4-carboxylic acid (PSI-421), a P-selectin inhibitor with improved pharmacokinetic properties and oral efficacy in models of vascular injury. J Med Chem 2010; 53(16):6003-17.
131. Ushiyama S, Laue T M, Moore K L, Erickson H P, McEver R P. Structural and functional characterization of monomeric soluble P-selectin and comparison with membrane P-selectin. J Biol Chem 1993; 268(20):15229-37.
132. Mehta P, Cummings R D, McEver R P. Affinity and kinetic analysis of P-selectin binding to P-selectin glycoprotein ligand-1. J Biol Chem 1998; 273(49):32506-13.
133. Japp A G, Chelliah R, Tattersall L, Lang N N, Meng X, Weisel K, Katz A, Burt D, Fox K A, Feuerstein G Z, Connolly T M, Newby D E. Effect of PSI-697, a novel P-selectin inhibitor, on platelet-monocyte aggregate formation in humans. J Am Heart Assoc 2013; 2(1):e006007.
134. Telen M J, Wun T, McCavit T L, De Castro L M, Krishnamurti L, Lanzkron S, Hsu L L, Smith W R, Rhee S, Magnani J L, Thackray H. Randomized phase 2 study of GMI-1070 in SCD: Reduction in time to resolution of vaso-occlusive events and decreased opioid use. Blood 2015; 125(17):2656-64.
135. Dowdy S F. Overcoming cellular barriers for RNA therapeutics. Nat Biotechnol 2017; 35(3):222-9.
136. Fosgerau K, Hoffmann T. Peptide therapeutics: Current status and future directions. Drug Discov Today 2015; 20(1):122-8.
137. Lau J L, Dunn M K. Therapeutic peptides: Historical perspectives, current development trends, and future directions. Bioorg Med Chem 2018; 26(10):2700-7.
138. Park E J, Choi J, Lee K C, Na D H. Emerging PEGylated non-biologic drugs. Expert Opin Emerg Drugs 2019; 24(2):107-19.
139. Liao D S, Grossi F V, El Mehdi D, Gerber M R, Brown D M, Heier J S, Wykoff C C, Singerman L J, Abraham P, Grassmann F, Nuernberg P, Weber B H F, Deschatelets P, Kim R Y, Chung C Y, Ribeiro R M, Hamdani M, Rosenfeld P J, Boyer D S, Slakter J S, Francois C G. Complement C3 inhibitor pegcetacoplan for geographic atrophy secondary to age-related macular degeneration: A randomized phase 2 trial. Ophthalmology 2020; 127(2):186-95.
140. Diaz J A, Saha P, Cooley B, Palmer O R, Grover S P, Mackman N, Wakefield T W, Henke P K, Smith A, Lal B K. Choosing a mouse model of venous thrombosis: A consensus assessment of utility and application. J Thromb Haemost 2019; 17(4):699-707.
141. Bray F, Ferlay J, Soerjomataram I, Siegel R L, Torre L A, Jemal A. Global cancer statistics 2018: GLOBOCAN estimates of incidence and mortality worldwide for 36 cancers in 185 countries. C A Cancer J Clin 2018; 68(6):394-424.
142. Kearon C, Akl E A, Ornelas J, Blaivas A, Jimenez D, Bounameaux H, Huisman M, King C S, Morris T A, Sood N, Stevens S M, Vintch J R E, Wells P, Woller S C, Moores L. Antithrombotic therapy for VTE disease: CHEST guideline and expert panel report. Chest 2016; 149(2):315-52.

### Embodiments

1. A glycopeptide, or a salt thereof, comprising the formula Y¹X¹Y²X²X³Y³X⁴X⁵X⁶Z¹X⁷W¹ (SEQ ID NO: 1), wherein:
   W¹ is threonine or serine conjugated with a saccharide or polysaccharide via a linker L¹;
   L¹ comprises a substituted or unsubstituted aryl group or a substituted or unsubstituted heteroaryl group;
   X¹, X², X³, X⁴, X⁵, X⁶, and X⁷ are each individually and independently any amino acid;
   Y¹, Y², and Y³ are each individually and independently tyrosine, phenylalanine, or phenylglycine, and wherein Y¹, Y², and Y³ are each independently unsubstituted or substituted with —SO₃H, —CH₂SO₃H, —CF₂SO₃H, —CO₂H, —CONH₂, —NHSO₂CH₃, —SO₂NH₂, or —CH₂PO₃H;
   wherein at least one of Y¹, Y², and Y³ is substituted with —CH₂SO₃H; and
   Z¹ is proline or hydroxyproline.
2. The glycopeptide of embodiment 1, or a salt thereof, wherein the saccharide or polysaccharide comprises one or more sugars selected from the group consisting of: 2-(acetylamino)-2-deoxy-galactose, galactose, 2-(acetylamino)-2-deoxy-glucose, fucose, and 5-acetamido-3,5-dideoxy-glycero-galacto-2-nonulosonic acid.
3. The glycopeptide of any of the preceding embodiments, or a salt thereof, wherein at least two of Y¹, Y², and Y³ are substituted with —CH₂SO₃H.
4. The glycopeptide of any of the preceding embodiments, or a salt thereof, wherein each of Y¹, Y², and Y³ is substituted with —CH₂SO₃H.
5. The glycopeptide of any of the preceding embodiments, or a salt thereof, wherein each of Y¹, Y², and Y³ is phenylglycine substituted with —CH₂SO₃H.
6. The glycopeptide of any of the preceding embodiments, or a salt thereof, wherein the polysaccharide is sialyl Lewis X or sialyl Lewis A.
7. The glycopeptide of any of the preceding embodiments, or a salt thereof, wherein W¹ is threonine.
8. The glycopeptide of any of the preceding embodiments, or a salt thereof, wherein W¹ is serine.
9. The glycopeptide of any of the preceding embodiments, or a salt thereof, wherein the polysaccharide comprises a radical S:

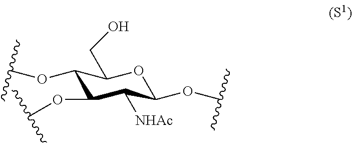

and wherein L¹ is bonded to the anomeric oxygen of S¹.
10. The glycopeptide of embodiment 9, or a salt thereof, wherein the polysaccharide further comprises an α 1-3 bond between S¹ and a radical S²:

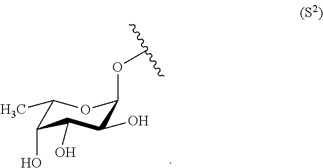

11. The glycopeptide of embodiment 10, or a salt thereof, wherein the polysaccharide further comprises a β 1-4 bond between S¹ and a radical S³:

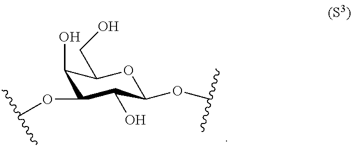

12. The glycopeptide of embodiment 11, or a salt thereof, wherein the polysaccharide further comprises a β 1-3 bond between S³ and a radical S⁴:

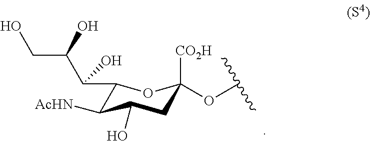

13. The glycopeptide of any of the preceding embodiments, or a salt thereof, wherein the polysaccharide is of the formula:

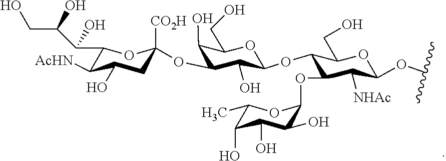

14. The glycopeptide of any of the preceding embodiments, or a salt thereof, wherein X¹, X³, X⁴, and X⁷ are each individually and independently E, D, N, or Q.
15. The glycopeptide of any of the preceding embodiments, or a salt thereof, wherein X², X⁵, and X⁶ are each individually and independently L, I, V, A or F.
16. The glycopeptide of any of the preceding embodiments, or a salt thereof, wherein the glycopeptide comprises:

(SEQ ID NO: 2)
Y¹EY²LDY³DFLZ¹EW¹,
(SEQ ID NO: 3)
Y¹EY²LDY³DFLZ¹EW¹EP,
(SEQ ID NO: 4)
Y¹EY²LDY³DFLZ¹EW¹EPL,
(SEQ ID NO: 5)
EY¹EY²LDY³DFLZ¹EW¹,
(SEQ ID NO: 6)
EY¹EY²LDY³DFLZ¹EW¹E,
(SEQ ID NO: 7)
EY¹EY²LDY³DFLZ¹EW¹EP,
(SEQ ID NO: 8)
EY¹EY²LDY³DFLZ¹EW¹EPL,
(SEQ ID NO: 9)
KEY¹EY²LDY³DFLZ¹EW¹,
(SEQ ID NO: 10)
KEY¹EY²LDY³DFLZ¹EW¹E,
(SEQ ID NO: 11)
KEY¹EY²LDY³DFLZ¹EW¹EP,
or
(SEQ ID NO: 12)
KEY¹EY²LDY³DFLZ¹EW¹EPL.

17. The glycopeptide of any of the preceding embodiments, or a salt thereof, wherein L¹ comprises a substituted or unsubstituted phenyl group.
18. The glycopeptide of embodiment 17, or a salt thereof, wherein L¹ has the structure:

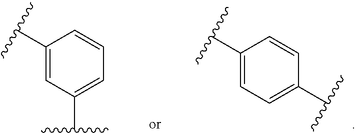 [SMILES: *c1cccc(*)c1 ; *c1ccc(*)cc1]

19. The glycopeptide of any of embodiments 1-16, or a salt thereof, wherein L¹ comprises a triazole.
20. The glycopeptide of embodiment 19, or a salt thereof, wherein the L¹ has the structure:

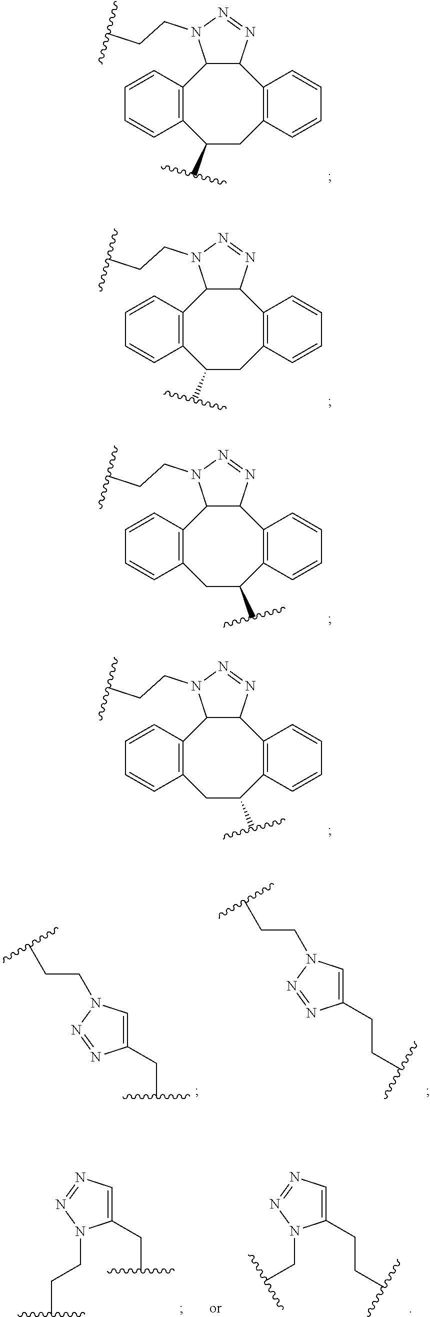

21. The glycopeptide of embodiment 19, or a salt thereof, wherein L¹ comprises a 1,2,3-triazole.
22. The glycopeptide, or salt thereof, of any of the preceding embodiments, comprising an N-terminal acetyl moiety.
23. The glycopeptide of embodiment 1, or a salt thereof, comprising the formula Ac-KEY¹EY²LDY³DFLZ¹EW¹EPL (SEQ ID NO: 13), wherein:
   W¹ is threonine conjugated with a polysaccharide via a linker L¹;
   L1 is

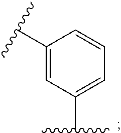 [SMILES: *c1cccc(*)c1]

and
Y¹, Y², and Y³ are each phenylalanine substituted with —CH₂SO₃H.
24. The glycopeptide of embodiment 1, or a salt thereof, comprising the formula Ac-KEY¹EY²LDY³DFLZ¹EW¹EPL (SEQ ID NO: 13), wherein:
   W¹ is threonine conjugated with a polysaccharide via a linker L¹;
   L¹ is

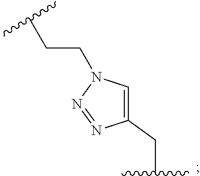 [SMILES: *CCn1cc(C*)nn1]

and
Y¹, Y², and Y³ are each phenylalanine substituted with —CH₂SO₃H.
25. The glycopeptide of embodiment 24, having the structure:

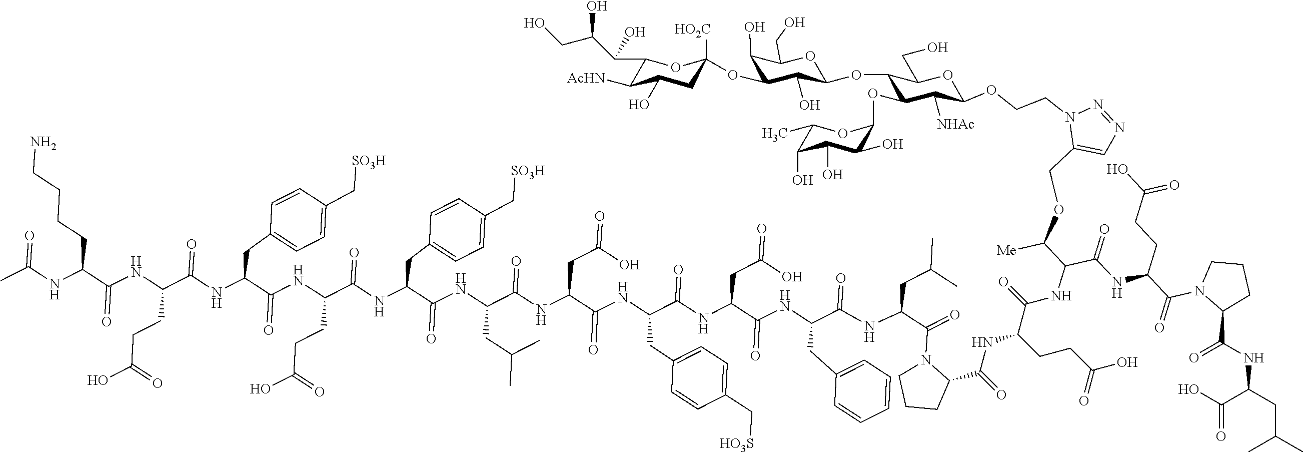

or a salt thereof.
26. The glycopeptide, or salt thereof, of any of the preceding embodiments, further comprising a substituted or unsubstituted aliphatic moiety, or a substituted or unsubstituted heteroaliphatic moiety.
27. The glycopeptide, or salt thereof, of embodiment 26, wherein the aliphatic moiety is a substituted or unsubstituted C₆-C₂₀ alkyl moiety.
28. The glycopeptide, or salt thereof, of embodiment 26, wherein the aliphatic moiety is palymitoyl.
29. The glycopeptide, or salt thereof, of embodiment 26, wherein the heteroaliphatic moiety is polyethylene glycol (PEG).
30. The glycopeptide of embodiment 29, having the structure:

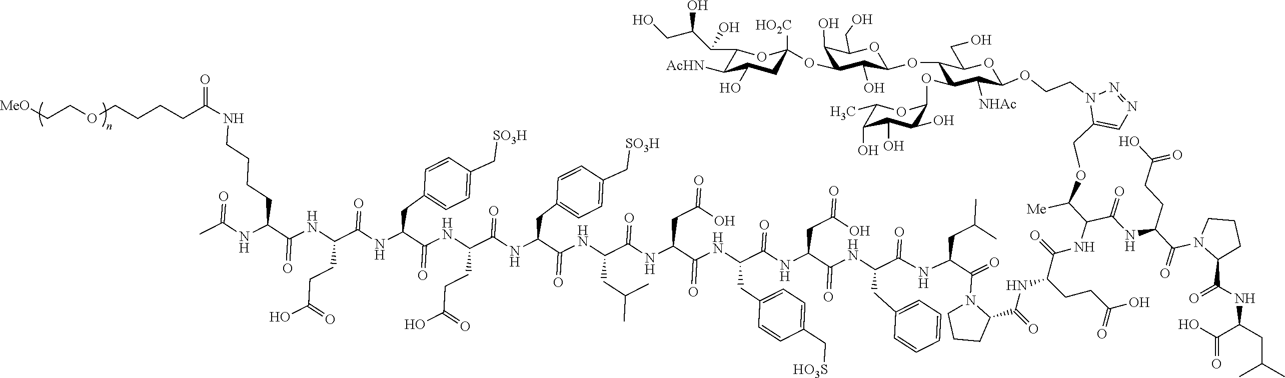

or a salt thereof; wherein n is 1-10,000.
31. The glycopeptide, or salt thereof, of embodiment 30, wherein n is about 903.
32. A pharmaceutical composition comprising a glycopeptide of any of the preceding embodiments, or a salt thereof, and a pharmaceutically acceptable excipient.
33. The pharmaceutical composition of embodiment 32, further comprising an additional therapeutic agent.
34. The pharmaceutical composition of embodiment 32 or 33, which is formulated for intravenous delivery, or which is formulated for subcutaneous delivery.
35. A method, comprising administering to a subject a glycopeptide, or a salt thereof, of any of embodiments 1-31, or a pharmaceutical composition of any of embodiments 32-34.
36. A method of inhibiting P-selectin binding to PSGL-1, comprising contacting the P-selectin with a glycopeptide, or salt thereof, of any of embodiments 1-31.
37. A method of treating or preventing cardiovascular disease, atherosclerosis, atherosclerotic lesions, thrombus formation, thromboembolism, stroke, or myocardial infarction in a subject in need thereof, comprising administering to the subject an effective amount of a glycopeptide, or a salt thereof, of any of embodiments 1-31, or a pharmaceutical composition of any of embodiments 32-34.
38. The method of embodiment 37 wherein the subject is at risk of, exhibiting symptoms of, or diagnosed with atherosclerosis, atherosclerotic lesions, thrombus formation, thromboembolism, stroke, or myocardial infarction.
39. The method of embodiment 37, wherein the subject has an increased risk of bleeding relative to that of a healthy adult.
40. The method of embodiment 37, wherein the subject has a history of bleeding (e.g., bleeding requiring blood transfusion or hospitalization).
41. The method of embodiment 37, wherein the subject has a history of abnormal liver or kidney function, or has increased fall risk.
42. The method of any of embodiments 37-41, wherein the thromboembolism is venous thromboembolism (VTE).
43. The method of embodiment 42, wherein the VTE is cancer-associated.
44. A method of thromboprophylaxis, comprising administering to a subject diagnosed with cancer an effective amount of a glycopeptide, or a salt thereof, of any of embodiments 1-31, or a pharmaceutical composition of any of embodiments 32-34.
45. A method of treating or preventing allergy or lung disease in a subject in need thereof, comprising administering to the subject an effective amount of a glycopeptide, or a salt thereof, of any of embodiments 1-31, or a pharmaceutical composition of any of embodiments 32-34.
46. The method of embodiment 44 wherein the subject is at risk of, exhibiting symptoms of, or diagnosed with asthma, bronchitis, emphysema, and COPD.
47. A method of making a glycopeptide, comprising reacting an polyaccharide group comprising a first reactive moiety, with a peptide comprising a second reactive moiety, to obtain the glycopeptide;
   wherein the first reactive moiety and the second reactive moiety react to form a triazole containing moiety.
48. The method of embodiment 47, wherein the first reactive moiety comprises an azide or an alkyne.
49. The method of embodiment 47, wherein the polysaccharide group has the structure:

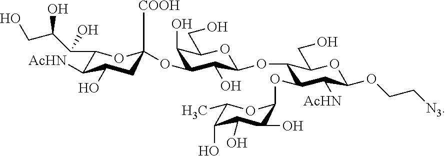

50. The method of embodiment 47, wherein the second reactive moiety comprises an azide or an alkyne.
51. The method of any of embodiments 48-50, wherein the peptide has the structure:

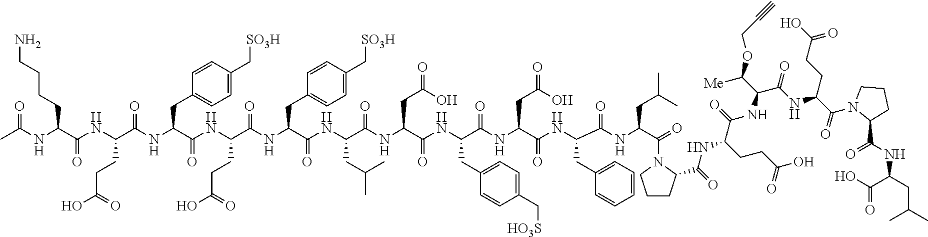 [SMILES: C#CCO[C@H](C)[C@H](NC(=O)[C@H](CCC(=O)O)NC(=O)[C@@H]1CCCN1C(=O)[C@H](CC(C)C)NC(=O)[C@H](Cc1ccccc1)NC(=O)[C@H](CC(=O)O)NC(=O)[C@H](Cc1ccc(CS(=O)(=O)O)cc1)NC(=O)[C@H](CC(=O)O)NC(=O)[C@H](CC(C)C)NC(=O)[C@H](Cc1ccc(CS(=O)(=O)O)cc1)NC(=O)[C@H](CCC(=O)O)NC(=O)[C@H](Cc1ccc(CS(=O)(=O)O)cc1)NC(=O)[C@H](CCC(=O)O)NC(=O)[C@H](CCCCN)NC(C)=O)C(=O)N[C@@H](CCC(=O)O)C(=O)N1CCC[C@H]1C(=O)N[C@@H](CC(C)C)C(=O)O]

52. A kit comprising a glycopeptide, or salt thereof, of any of embodiments 1-31, and instructions for use.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed was only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Furthermore, the present disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the present disclosure, or aspects of the present disclosure, is/are referred to as comprising particular elements and/or features, certain embodiments of the present disclosure or aspects of the present disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the present disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the present disclosure can be excluded from any claim, for any reason, whether or not related to the existence of prior art.

It should also be understood that, unless clearly indicated to the contrary, in any methods claimed herein that include more than one step or act, the order of the steps or acts of the method is not necessarily limited to the order in which the steps or acts of the method are recited.

1. A glycopeptide, or a salt thereof, comprising the formula Y¹X¹Y²X²X³Y³X⁴X⁵X⁶Z¹X⁷W¹ (SEQ ID NO: 1), wherein:
W¹ is threonine or serine conjugated with a saccharide or polysaccharide via a linker L¹;
L¹ comprises a substituted or unsubstituted aryl group or a substituted or unsubstituted heteroaryl group;
X¹, X², X³, X⁴, X⁵, X⁶, and X⁷ are each individually and independently any amino acid;
Y¹, Y², and Y³ are each individually and independently tyrosine, phenylalanine, or phenylglycine, and wherein Y¹, Y², and Y³ are each independently unsubstituted or substituted with —SO₃H, —CH₂SO₃H, —CF₂SO₃H, —CO₂H, —CONH₂, —NHSO₂CH₃, —SO₂NH₂, or —CH₂PO₃H;
wherein at least one of Y¹, Y², and Y³ is substituted with —CH₂SO₃H; and
Z¹ is proline or hydroxyproline.

2. The glycopeptide of claim 1, or a salt thereof, wherein the saccharide or polysaccharide comprises one or more sugars selected from the group consisting of: 2-(acetylamino)-2-deoxy-galactose, galactose, 2-(acetylamino)-2-deoxy-glucose, fucose, and 5-acetamido-3,5-dideoxy-glycero-galacto-2-nonulosonic acid.

3. The glycopeptide of any one of the preceding claims, or a salt thereof, wherein at least two of Y¹, Y², and Y³ are substituted with —CH₂SO₃H.

4. The glycopeptide of claim 3, or a salt thereof, wherein each of Y¹, Y², and Y³ is substituted with —CH₂SO₃H.

5. The glycopeptide of claim 4, or a salt thereof, wherein each of Y¹, Y², and Y³ is phenylglycine substituted with —CH₂SO₃H.

6. The glycopeptide of claim 1, or a salt thereof, wherein the polysaccharide is sialyl Lewis X or sialyl Lewis A.

7. The glycopeptide of claim 1, or a salt thereof, wherein W¹ is threonine.

8. The glycopeptide of claim 1, or a salt thereof, wherein W¹ is serine.

9. The glycopeptide of claim 1, or a salt thereof, wherein the polysaccharide comprises a radical S¹: 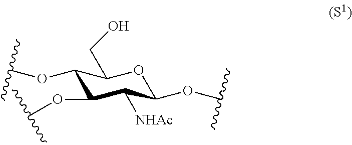
and wherein L¹ is bonded to the anomeric oxygen of S.

10. The glycopeptide of claim 9, or a salt thereof, wherein the polysaccharide further comprises an α 1-3 bond between S¹ and a radical S²:

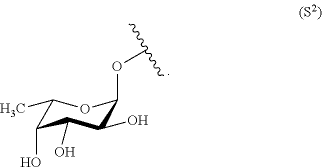

11. The glycopeptide of claim 10, or a salt thereof, wherein the polysaccharide further comprises a β 1-4 bond between S¹ and a radical S³:

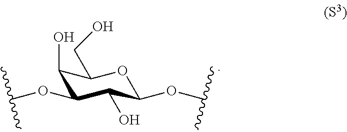

12. The glycopeptide of claim 11, or a salt thereof, wherein the polysaccharide further comprises a β 1-3 bond between S³ and a radical S⁴:

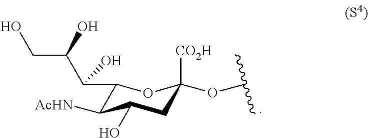

13. The glycopeptide of claim 12, or a salt thereof, wherein the polysaccharide is of the formula:

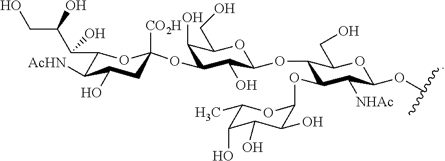

14. The glycopeptide of claim 1, or a salt thereof, wherein X¹, X³, X⁴, and X⁷ are each individually and independently E, D, N, or Q.

15. The glycopeptide of claim 1, or a salt thereof, wherein X², X⁵, and X⁶ are each individually and independently L, I, V, A or F.

16. The glycopeptide of claim 1, or a salt thereof, wherein the glycopeptide comprises:
(SEQ ID NO: 2)
Y¹EY²LDY³DFLZ¹EW¹,
(SEQ ID NO: 3)
Y¹EY²LDY³DFLZ¹EW¹EP,
(SEQ ID NO: 4)
Y¹EY²LDY³DFLZ¹EW¹EPL,
(SEQ ID NO: 5)
EY¹EY²LDY³DFLZ¹EW¹,
(SEQ ID NO: 6)
EY¹EY²LDY³DFLZ¹EW¹E,
(SEQ ID NO: 7)
EY¹EY²LDY³DFLZ¹EW¹EP,
(SEQ ID NO: 8)
EY¹EY²LDY³DFLZ¹EW¹EPL,
(SEQ ID NO: 9)
KEY¹EY²LDY³DFLZ¹EW¹,
(SEQ ID NO: 10)
KEY¹EY²LDY³DFLZ¹EW¹E,
(SEQ ID NO: 11)
KEY¹EY²LDY³DFLZ¹EW¹EP,
or
(SEQ ID NO: 12)
KEY¹EY²LDY³DFLZ¹EW¹EPL.

17. The glycopeptide of claim 1, or a salt thereof, wherein L¹ comprises a substituted or unsubstituted phenyl group.

18. The glycopeptide of claim 17, or a salt thereof, wherein L¹ has the structure:

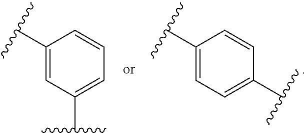 [SMILES: *c1cccc(*)c1 ; *c1ccc(*)cc1]

19. The glycopeptide of claim 1, or a salt thereof, wherein L¹ comprises a triazole.

20. The glycopeptide of claim 19, or a salt thereof, wherein the L¹ has the structure:

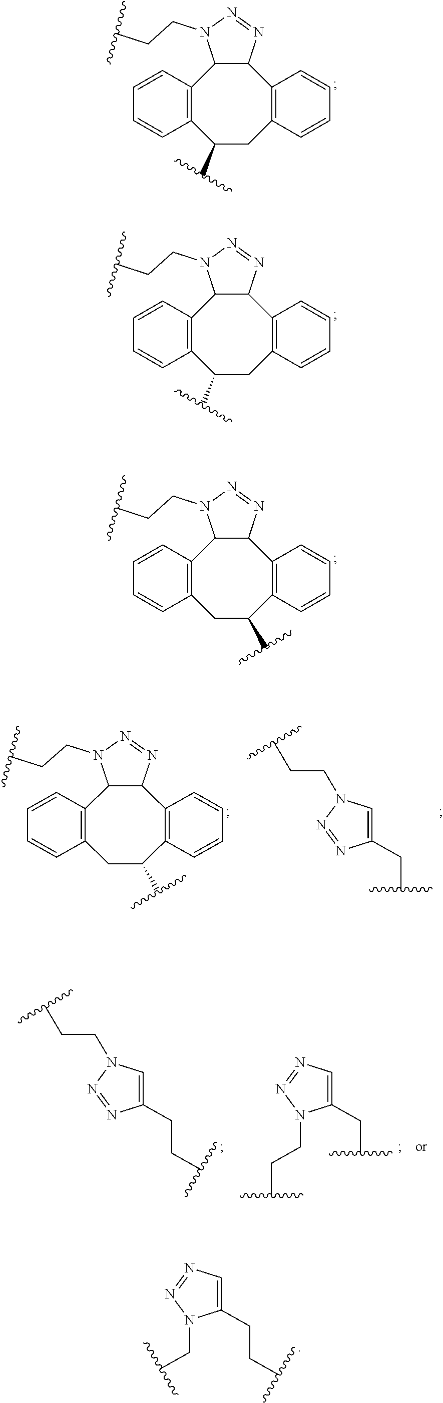

21. The glycopeptide of claim 19, or a salt thereof, wherein L¹ comprises a 1,2,3-triazole.

22. The glycopeptide, or salt thereof, of claim 1, comprising an N-terminal acetyl moiety.

23. The glycopeptide of claim 1, or a salt thereof, comprising the formula Ac-KEY¹EY²LDY³DFLZ¹EW¹EPL (SEQ ID NO: 13), wherein:
W¹ is threonine conjugated with a polysaccharide via a linker L¹;
L¹ is 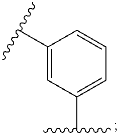 [SMILES: *c1cccc(*)c1]
and
Y¹, Y², and Y³ are each phenylalanine substituted with —CH₂SO₃H.

24. The glycopeptide of claim 1, or a salt thereof, comprising the formula Ac-KEY¹EY²LDY³DFLZ¹EW¹EPL (SEQ ID NO: 13), wherein:
W¹ is threonine conjugated with a polysaccharide via a linker L¹;
L¹ is 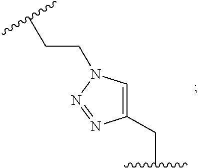 [SMILES: *CCn1cc(C*)nn1]
and
Y¹, Y², and Y³ are each phenylalanine substituted with —CH₂SO₃H.

25. The glycopeptide of claim 24, having the structure: 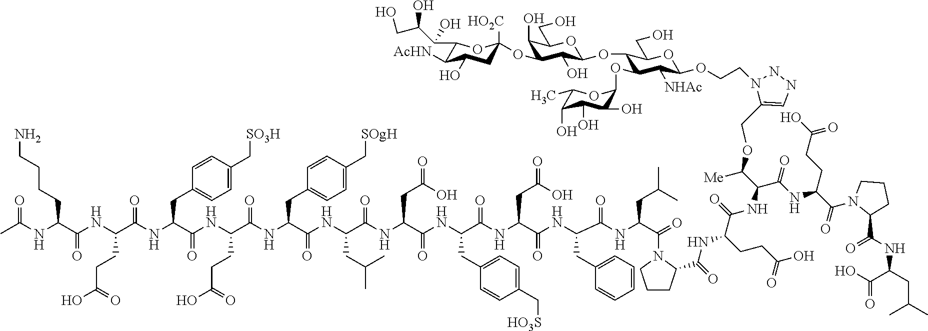
or a salt thereof.

26. The glycopeptide, or salt thereof, of claim 1, further comprising a substituted or unsubstituted aliphatic moiety, or a substituted or unsubstituted heteroaliphatic moiety.

27. The glycopeptide, or salt thereof, of claim 26, wherein the aliphatic moiety is a substituted or unsubstituted C₆-C₂₀ alkyl moiety.

28. The glycopeptide, or salt thereof, of claim 26, wherein the aliphatic moiety is palymitoyl.

29. The glycopeptide, or salt thereof, of claim 26, wherein the heteroaliphatic moiety is polyethylene glycol (PEG).

30. The glycopeptide of claim 29, having the structure: 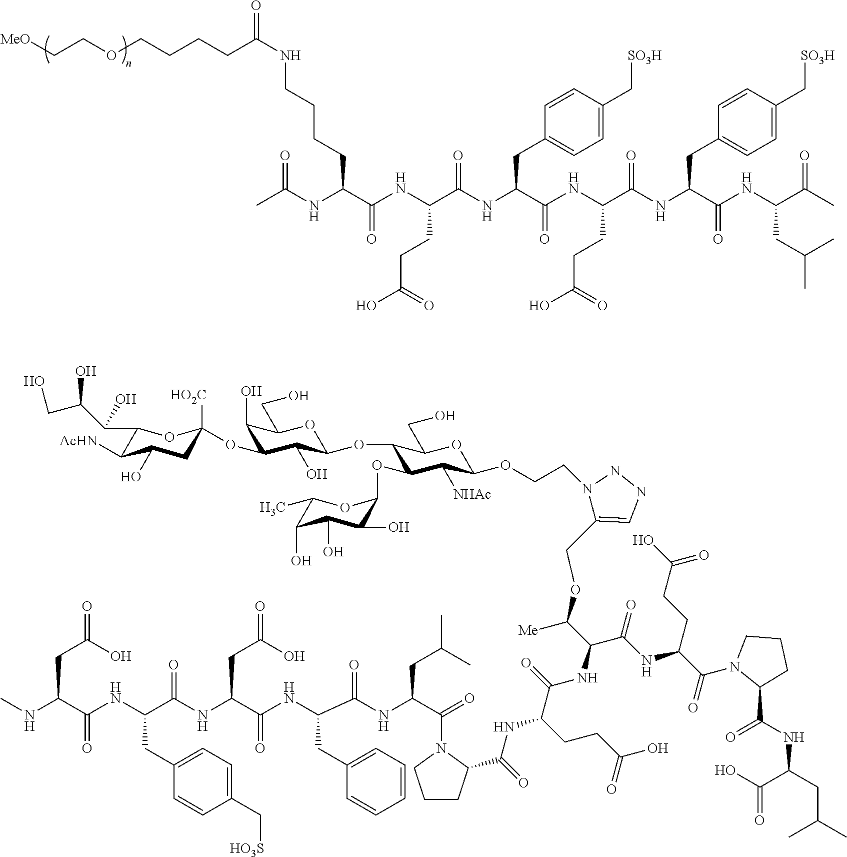
or a salt thereof; wherein n is 1-10,000.

31. The glycopeptide, or salt thereof, of claim 30, wherein n is about 903.

32. A pharmaceutical composition comprising a glycopeptide of claim 1, or a salt thereof, and a pharmaceutically acceptable excipient.

33. The pharmaceutical composition of claim 32, further comprising an additional therapeutic agent.

34. The pharmaceutical composition of claim 32, which is formulated for intravenous delivery.

35. A method comprising administering to a subject a glycopeptide, or a salt thereof, of any one of claims 1-31, or a pharmaceutical composition of any one of claims 32-34.

36. A method of inhibiting P-selectin binding to PSGL-1, comprising contacting the P-selectin with a glycopeptide, or salt thereof, of any one of claims 1-31.

37. A method of treating or preventing cardiovascular disease, atherosclerosis, atherosclerotic lesions, thrombus formation, thromboembolism, stroke, or myocardial infarction in a subject in need thereof, comprising administering to the subject an effective amount of a glycopeptide, or a salt thereof, of any one of claims 1-31, or a pharmaceutical composition of any one of claims 32-34.

38. The method of claim 37 wherein the subject is at risk of, exhibiting symptoms of, or diagnosed with atherosclerosis, atherosclerotic lesions, thrombus formation, thromboembolism, stroke, or myocardial infarction.

39. The method of claim 37, wherein the subject has an increased risk of bleeding relative to that of a healthy adult.

40. The method of claim 37, wherein the subject has a history of bleeding.

41. The method of claim 37, wherein the subject has a history of abnormal liver or kidney function, or has increased fall risk.

42. The method of claim 37, wherein the thromboembolism is venous thromboembolism (VTE).

43. The method of claim 42, wherein the VTE is cancer-associated.

44. A method of thromboprophylaxis, comprising administering to a subject diagnosed with cancer an effective amount of a glycopeptide, or a salt thereof, of any one of claims 1-31, or a pharmaceutical composition of any one of claims 32-34.

45. A method of treating or preventing allergy or lung disease in a subject in need thereof, comprising administering to the subject an effective amount of a glycopeptide, or a salt thereof, of any one of claims 1-31, or a pharmaceutical composition of any one of claims 32-34.

46. The method of claim 44 wherein the subject is at risk of, exhibiting symptoms of, or diagnosed with asthma, bronchitis, emphysema, and COPD.

47. A method of making a glycopeptide, comprising reacting an polyaccharide group comprising a first reactive moiety, with a peptide comprising a second reactive moiety, to obtain the glycopeptide;
wherein the first reactive moiety and the second reactive moiety react to form a triazole containing moiety.

48. The method of claim 47, wherein the first reactive moiety comprises an azide or an alkyne.

49. The method of claim 47, wherein the polysaccharide group has the structure:

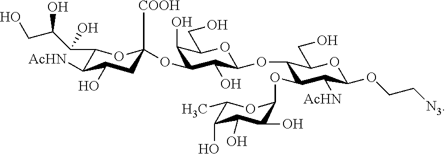

50. The method of claim 47, wherein the second reactive moiety comprises an azide or an alkyne.

51. The method of any one of claims 48-50, wherein the peptide has the structure:

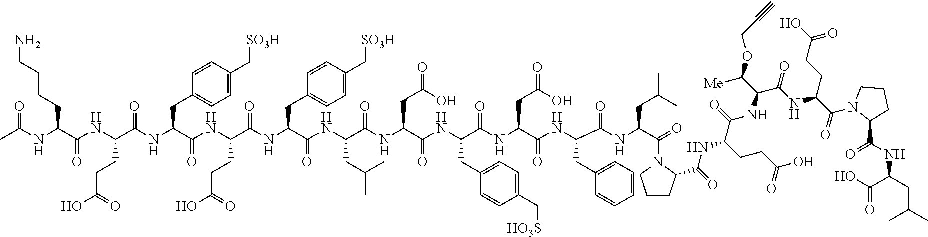 [SMILES: C#CCO[C@H](C)[C@H](NC(=O)[C@H](CCC(=O)O)NC(=O)[C@@H]1CCCN1C(=O)[C@H](CC(C)C)NC(=O)[C@H](Cc1ccccc1)NC(=O)[C@H](CC(=O)O)NC(=O)[C@H](Cc1ccc(CS(=O)(=O)O)cc1)NC(=O)[C@H](CC(=O)O)NC(=O)[C@H](CC(C)C)NC(=O)[C@H](Cc1ccc(CS(=O)(=O)O)cc1)NC(=O)[C@H](CCC(=O)O)NC(=O)[C@H](Cc1ccc(CS(=O)(=O)O)cc1)NC(=O)[C@H](CCC(=O)O)NC(=O)[C@H](CCCCN)NC(C)=O)C(=O)N[C@@H](CCC(=O)O)C(=O)N1CCC[C@H]1C(=O)N[C@@H](CC(C)C)C(=O)O]

52. A kit comprising a glycopeptide, or salt thereof, of any one of claims 1-31, and instructions for use.


SEQUENCE LISTING (26 sequences)

SEQ ID NO: 1 - skipped

SEQ ID NO: 2 - protein, 12 aa, synthetic construct
  VARIANT 1: note = May be tyrosine, phenylalanine, or phenylglycine
  VARIANT 3: note = May be tyrosine, phenylalanine, or phenylglycine
  VARIANT 6: note = May be tyrosine, phenylalanine, or phenylglycine
  VARIANT 10: note = May be proline or hydroxyproline
  VARIANT 12: note = May be threonine or serine conjugated with a saccharide or polysaccharide via a linker
XEXLDXDFLXEX

SEQ ID NO: 3 - protein, 14 aa, synthetic construct
  VARIANT 1: note = May be tyrosine, phenylalanine, or phenylglycine
  VARIANT 3: note = May be tyrosine, phenylalanine, or phenylglycine
  VARIANT 6: note = May be tyrosine, phenylalanine, or phenylglycine
  VARIANT 10: note = May be proline or hydroxyproline
  VARIANT 12: note = May be threonine or serine conjugated with a saccharide or polysaccharide via a linker
XEXLDXDFLXEXEP

SEQ ID NO: 4 - protein, 15 aa, synthetic construct
  VARIANT 1: note = May be tyrosine, phenylalanine, or phenylglycine
  VARIANT 3: note = May be tyrosine, phenylalanine, or phenylglycine
  VARIANT 6: note = May be tyrosine, phenylalanine, or phenylglycine
  VARIANT 10: note = May be proline or hydroxyproline
  VARIANT 12: note = May be threonine or serine conjugated with a saccharide or polysaccharide via a linker
XEXLDXDFLXEXEPL

SEQ ID NO: 5 - protein, 13 aa, synthetic construct
  VARIANT 2: note = May be tyrosine, phenylalanine, or phenylglycine
  VARIANT 4: note = May be tyrosine, phenylalanine, or phenylglycine
  VARIANT 7: note = May be tyrosine, phenylalanine, or phenylglycine
  VARIANT 11: note = May be proline or hydroxyproline
  VARIANT 13: note = May be threonine or serine conjugated with a saccharide or polysaccharide via a linker
EXEXLDXDFLXEX

SEQ ID NO: 6 - protein, 14 aa, synthetic construct
  VARIANT 2: note = May be tyrosine, phenylalanine, or phenylglycine
  VARIANT 4: note = May be tyrosine, phenylalanine, or phenylglycine
  VARIANT 7: note = May be tyrosine, phenylalanine, or phenylglycine
  VARIANT 11: note = May be proline or hydroxyproline
  VARIANT 13: note = May be threonine or serine conjugated with a saccharide or polysaccharide via a linker
EXEXLDXDFLXEXE

SEQ ID NO: 7 - protein, 15 aa, synthetic construct
  VARIANT 2: note = May be tyrosine, phenylalanine, or phenylglycine
  VARIANT 4: note = May be tyrosine, phenylalanine, or phenylglycine
  VARIANT 7: note = May be tyrosine, phenylalanine, or phenylglycine
  VARIANT 11: note = May be proline or hydroxyproline
  VARIANT 13: note = May be threonine or serine conjugated with a saccharide or polysaccharide via a linker
EXEXLDXDFLXEXEP

SEQ ID NO: 8 - protein, 16 aa, synthetic construct
  VARIANT 2: note = May be tyrosine, phenylalanine, or phenylglycine
  VARIANT 4: note = May be tyrosine, phenylalanine, or phenylglycine
  VARIANT 7: note = May be tyrosine, phenylalanine, or phenylglycine
  VARIANT 11: note = May be proline or hydroxyproline
  VARIANT 13: note = May be threonine or serine conjugated with a saccharide or polysaccharide via a linker
EXEXLDXDFLXEXEPL

SEQ ID NO: 9 - protein, 14 aa, synthetic construct
  VARIANT 3: note = May be tyrosine, phenylalanine, or phenylglycine
  VARIANT 5: note = May be tyrosine, phenylalanine, or phenylglycine
  VARIANT 8: note = May be tyrosine, phenylalanine, or phenylglycine
  VARIANT 12: note = May be proline or hydroxyproline
  VARIANT 14: note = May be threonine or serine conjugated with a saccharide or polysaccharide via a linker
KEXEXLDXDFLXEX

SEQ ID NO: 10 - protein, 15 aa, synthetic construct
  VARIANT 3: note = May be tyrosine, phenylalanine, or phenylglycine
  VARIANT 5: note = May be tyrosine, phenylalanine, or phenylglycine
  VARIANT 8: note = May be tyrosine, phenylalanine, or phenylglycine
  VARIANT 12: note = May be proline or hydroxyproline
  VARIANT 14: note = May be threonine or serine conjugated with a saccharide or polysaccharide via a linker
KEXEXLDXDFLXEXE

SEQ ID NO: 11 - protein, 16 aa, synthetic construct
  VARIANT 3: note = May be tyrosine, phenylalanine, or phenylglycine
  VARIANT 5: note = May be tyrosine, phenylalanine, or phenylglycine
  VARIANT 8: note = May be tyrosine, phenylalanine, or phenylglycine
  VARIANT 12: note = May be proline or hydroxyproline
  VARIANT 14: note = May be threonine or serine conjugated with a saccharide or polysaccharide via a linker
KEXEXLDXDFLXEXEP

SEQ ID NO: 12 - protein, 17 aa, synthetic construct
  VARIANT 3: note = May be tyrosine, phenylalanine, or phenylglycine
  VARIANT 5: note = May be tyrosine, phenylalanine, or phenylglycine
  VARIANT 8: note = May be tyrosine, phenylalanine, or phenylglycine
  VARIANT 12: note = May be proline or hydroxyproline
  VARIANT 14: note = May be threonine or serine conjugated with a saccharide or polysaccharide via a linker
KEXEXLDXDFLXEXEPL

SEQ ID NO: 13 - protein, 17 aa, synthetic construct
KEYEYLDYDFLZEWEPL

SEQ ID NO: 14 - protein, 17 aa, synthetic construct
KEFEFLDFDFLPETEPL

SEQ ID NO: 15 - protein, 18 aa, synthetic construct
  source 1..18: note = 4GSP-1
EYEYLDYDFLPETEPPEM

SEQ ID NO: 16 - protein, 18 aa, synthetic construct
  source 1..18: note = 4GSP-6
EYEYLDYDFLPETEPPEM

SEQ ID NO: 17 - protein, 17 aa, synthetic construct
  source 1..17: note = GSnP-3
KEFEFLDFDFLPETEPL

SEQ ID NO: 18 - protein, 17 aa, synthetic construct
  source 1..17: note = GSnP-4
KEFEFLDFDFLPETEPL

SEQ ID NO: 19 - protein, 17 aa, synthetic construct
  source 1..17: note = GSnP-5
KEFEFLDFDFLPETEPL

SEQ ID NO: 20 - protein, 17 aa, synthetic construct
  source 1..17: note = GSnP-5b
KEFEFLDFDFLPETEPL

SEQ ID NO: 21 - protein, 17 aa, synthetic construct
  source 1..17: note = GSnP-6
KEFEFLDFDFLPETEPL

SEQ ID NO: 22 - protein, 17 aa, synthetic construct
  source 1..17: note = GSnP-7
KEFEFLDFDFLPETEPL

SEQ ID NO: 23 - protein, 17 aa, synthetic construct
  source 1..17: note = G1
KEFEFLDFDFLPETEPL

SEQ ID NO: 24 - protein, 17 aa, synthetic construct
  source 1..17: note = G2
KEFEFLDFDFLPETEPL

SEQ ID NO: 25 - protein, 17 aa, synthetic construct
  source 1..17: note = G3
KEFEFLDFDFLPETEPL

SEQ ID NO: 26 - protein, 17 aa, synthetic construct
  source 1..17: note = G4
KEFEFLDFDFLPETEPL